(19) United States
(12) Patent Application Publication
(10) Pub. No.: US 2006/0036093 A1
(43) Pub. Date: Feb. 16, 2006

(54) Pyrimidinone compounds

(75) Inventors: Chu-Chung Lin, Taichung (TW); Hong-Chuan Chen, Taipei County (TW); Kuang-Yuan Lee, Hsinchu City (TW); Ying-Huey Huang, Changhua (TW); Yang-Ping Fan, Taipei City (TW); Yibin Xiang, Acton, MA (US)

Correspondence Address: FISH & RICHARDSON PC, P.O. BOX 1022, MINNEAPOLIS, MN, 55440-1022, US

(73) Assignee: TAIGEN BIOTECHNOLOGY

(21) Appl. No.: 11/204,792

(22) Filed: Aug. 16, 2005

Related U.S. Application Data
(60) Provisional application No. 60/601,776, filed on Aug. 16, 2004.

(51) Int. Cl. A61K 31/519; C07D 239/72; C07D 487/02

(52) U.S. Cl. 544/279; 544/283

(57) ABSTRACT

This invention relates to treating inflammatory and immune diseases with certain pyrimidinone compounds that bind to CXCR3 receptors. The pyrimidinone compounds are covered by the formula (I) shown below. Each variable is defined in the specification.

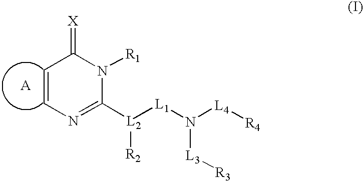

### CROSS REFERENCE TO RELATED APPLICATION

Pursuant to 35 U.S.C. § 119(e), this application claims priority to U.S. Provisional Application Ser. No. 60/601,776, filed Aug. 16, 2004, the contents of which are hereby incorporated by reference.

### BACKGROUND

Chemokines have been classified into four groups according to their structures. CXC and CC chemokines, the two largest groups, feature the presence and absence of an amino acid, respectively, between the first two cysteine residues in a conserved four-cysteine motif (Mackay C. R., Nat. Immunol., (2001) 2:95; Olson et al., Am. J. Physiol. Regul. Integr. Comp. Physiol., (2002) 283:R7). CXCR3 is the first chemokine receptor found to be highly induced by T cell activation (Loetscher et al., J. Exp. Med., (1996) 184:963). CXCR3 is expressed on some circulating blood T cells, B cells, and natural killer cells (Qin et al., J. Clin. Invest., (1998) 101:746). For example, expression of CXCR3 is induced virtually by all T cells in synovial fluid of rheumatoid arthritis and in various inflamed tissues (e.g., ulcerative colitis, chronic vaginitis, and sarcoidosis), particularly in perivascular regions. However, few T cells in normal lymph nodes are induced to express CXCR3 (Agostini et al., J. Immunol., (1998) 161:6413). Expression and responsiveness of CXCR3 can be markedly increased by T cell activation (Rabin et al., J. Immunol., (1999) 162:3840). CXCR3 is also consistently detected in functional forms on transformed B cells obtained from chronic lymphocytic leukemia patients (Trentin et al., J. Clin. Invest., (1999) 104:115).

CXCR3 binds to three highly potent, inflammation-inducible, ELR-negative CXC chemokines, i.e., I-TAC, Mig, and IP-10. These three chemokines chemoattract and induce calcium influx in activated T cells, tumor-infiltrating lymphocytes, and CXCR3-transfected cells (Loetscher et al., Eur. J. Immunol., (1998) 28:3696; Cole et al., J. Exp. Med., (1998) 187:2009; Weng et al., J. Biol. Chem., (1998) 273:18288). CXCR3 signaling appears to be an important mechanism for selective homing of activated/effector cells, which are known to accumulate preferentially at inflammatory sites and in many tumors. For example, IP-10 is expressed abundantly at various inflammatory sites, particularly those characterized by T cell infiltration, such as in tissues affected by delayed type hypersensitivity responses, experimental autoimmune encephalomyelitis, or a transplant undergoing rejection (Qin et al., J. Clin. Invest., (1998) 101:746). CXCR3 ligand-induced recruitment of leukocytes is thought to be an essential step in the pathogenesis of tissue-specific autoimmune inflammatory diseases, as well as in graft rejection (Hancock et al., J. Exp. Med., (2000) 192:1515).

### SUMMARY

This invention is based on the discovery that certain pyrimidinone compounds are unexpectedly effective in treating inflammatory and immune diseases through their binding to CXCR3 receptors.

In one aspect, this invention features pyrimidinone compounds of formula (I): 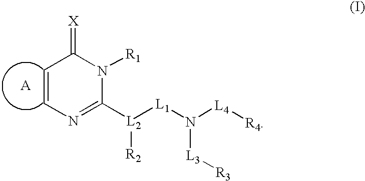
In this formula, A is aryl or heteroaryl; X is S or NRₐ₁; L₁ is —C(R_{b1}R_{b2})—, C₂-C₁₀ alkylene, C₂-C₁₀ heteroalkylene, or deleted; L₂ is 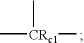
or L₂ and R₂ together are deleted; each of L₃ and L₄, independently, is —C(O)—, —SO₂—, —C(O)O—, —C(O)NR_{d1}—, —C(O)CH₂—, —CH₂C(O)—, —SO₂CH₂—, —CH₂SO₂—, C₁-C₁₀ alkylene, or C₁-C₁₀ heteroalkylene; or L₃, L₄, and the nitrogen atom to which they are both attached, together are C₅-C₇ heterocycloalkyl or heteroaryl; or L₁, L₃, and the nitrogen atom to which they are both attached, together are C₅-C₇ heterocycloalkyl or heteroaryl; or L₁, L₄, and the nitrogen atom to which they are both attached, together are C₅-C₇ heterocycloalkyl or heteroaryl; R₁ is H, C₁-C₁₀ alkyl, C₃-C₂₀ cycloalkyl, C₃-C₂₀ heterocycloalkyl, aryl, or heteroaryl; R₂ is H, C₁-C₁₀ alkyl, C₃-C₂₀ cycloalkyl, C₃-C₂₀ heterocycloalkyl, aryl, heteroaryl, or ORₑ₁; or R₂ and L₂ together are deleted; and each of R₃ and R₄, independently, is C₁-C₁₀ alkyl, C₃-C₂₀ cycloalkyl, C₃-C₂₀ heterocycloalkyl, aryl, heteroaryl, halo, cyano, amidino, guanidine, ureido, OR_{f1}, NR_{f1}R_{f2}, C(O)NR_{f1}R_{f2}, N(R_{f1})—C(O)R_{f2}, N(R_{f1})—C(O)OR_{f2}, C(O)R_{f1}, N(R_{f1})—C(S)NR_{f2}R_{f3}, N(R_{f1})—C(NR_{f2})—NR_{f3}R_{f4}, or N(R_{f1})—C(NR_{f2})—SR_{f3}; in which each of Rₐ₁, R_{b1}, R_{b2}, Rₑ₁, R_{d1}, Rₑ₁, R_{f1}, R_{f2}, R_{f3}, and R_{f4}, independently, is H, C₁-C₁₀ alkyl, C₃-C₂₀ cycloalkyl, C₃-C₂₀ heterocycloalkyl, aryl, heteroaryl, cyano, OR, COOR, or C(O)NH₂; or R_{b1}, R_{b2}, and the carbon atom to which they are both attached, together are C₃-C₈ cycloalkyl or C₃-C₈ heterocycloalkyl; or R_{c1}, R₂, and the carbon atom to which they are both attached, together are C₃-C₈ cycloalkyl or C₃-C₈ heterocycloalkyl; R being H or C₁-C₁₀ alkyl.

Referring to formula (I), a subset of the pyrimidinone compounds described above are those in which A is phenyl or thienyl; each of L₃ and L₄, independently, is —C(O)—, —CH₂—, —(CH₂)₂—, or —(CH₂)₃—; R₁ is phenyl substituted with F, OCH₃, or OCH₂CH₃; R₂ is methyl; one of R₃ and R₄ is methyl substituted with phenyl, in which the phenyl is further substituted with F, Cl, CF₃, or phenyl; and the other of R₃ and R₄ is C₃-C₂₀ heterocycloalkyl, heteroaryl, or NR_{f1}R_{f2}.

The term “alkyl” refers to a saturated or unsaturated, linear or branched hydrocarbon moiety, such as —CH₃, —CH₂—CH═CH₂, or branched —C₃H₇. The term “heteroalkyl” refers to an alkyl moiety having at least one heteroatom (e.g., N, O, or S). The term “alkylene” refers to a divalent, saturated or unsaturated, linear or branched hydrocarbon moiety, such as —CH₂— or —CH═CH—. The term “heteroalkylene” refers to an alkylene moiety having at least one heteroatom (e.g., N, O, or S). The term “cycloalkyl” refers to a saturated or unsaturated, non-aromatic, cyclic hydrocarbon moiety, such as cyclohexyl or cyclohexen-3-yl. The term “heterocycloalkyl” refers to a saturated or unsaturated, non-aromatic, cyclic moiety having at least one ring heteroatom (e.g., N, O, or S), such as 4-tetrahydropyranyl or 4-pyranyl. The term “aryl” refers to a hydrocarbon moiety having one or more aromatic rings. Examples of an aryl moiety include phenyl, phenylene, naphthyl, naphthylene, pyrenyl, anthryl, and phenanthryl. The term “heteroaryl” refers to a moiety having one or more aromatic rings that contain at least one heteroatom (e.g., N, O, or S). Examples of a heteroaryl moiety include furyl, furylene, fluorenyl, pyrrolyl, thienyl, oxazolyl, imidazolyl, thiazolyl, pyridyl, pyrimidinyl, quinazolinyl, quinolyl, isoquinolyl and indolyl.

Alkyl, heteroalkyl, alkylene, heteroalkylene, cycloalkyl, heterocycloalkyl, aryl, and heteroaryl mentioned herein include both substituted and unsubstituted moieties, unless specified otherwise. Possible substituents on cycloalkyl, heterocycloalkyl, aryl, and heteroaryl include C₁-C₁₀ alkyl, C₂-C₁₀ alkenyl, C₂-C₁₀ alkynyl, C₃-C₈ cycloalkyl, C₅-C₈ cycloalkenyl, C₁-C₁₀ alkoxy, aryl, aryloxy, heteroaryl, heteroaryloxy, amino, C₁-C₁₀ alkylamino, C₁-C₂₀ dialkylamino, arylamino, diarylamino, hydroxyl, halogen, thio, C₁-C₁₀ alkylthio, arylthio, C₁-C₁₀ alkylsulfonyl, arylsulfonyl, acylamino, aminoacyl, amidino, guanidine, ureido, cyano, nitro, acyl, acyloxy, carboxyl, and carboxylic ester. On the other hand, possible substituents on alkyl, heteroalkyl, alkylene, or heteroalkylene include all of the above-recited substituents except C₁-C₁₀ alkyl, C₂-C₁₀ alkenyl, and C₂-C₁₀ alkynyl. Cycloalkyl, heterocycloalkyl, aryl, and heteroaryl can also be fused with each other.

In another aspect, this invention features pyrimidinone compounds of formula (I) shown above in which A is aryl or heteroaryl; X is O, S, or NRₐ₁; L₁ is —C(R_{b1}R_{b2})—, C₂-C₁₀ alkylene, C₂-C₁₀ heteroalkylene, or deleted; L₂ is 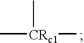
each of L₃ and L₄, independently, is —C(O)—, —SO₂—, —C(O)O—, —C(O)NR_{d1}—, —C(O)CH₂—, —CH₂C(O)—, —SO₂CH₂—, —CH₂SO₂—, C₁-C₁₀ alkylene, or C₁-C₁₀ heteroalkylene; or L₃, L₄, and the nitrogen atom to which they are both attached, together are C₅-C₇ heterocycloalkyl or heteroaryl; or L₁, L₃, and the nitrogen atom to which they are both attached, together are C₅-C₇ heterocycloalkyl or heteroaryl; or L₁, L₄, and the nitrogen atom to which they are both attached, together are C₅-C₇ heterocycloalkyl or heteroaryl; R₁ is H, C₁-C₁₀ alkyl, C₃-C₂₀ cycloalkyl, C₃-C₂₀ heterocycloalkyl, aryl, or heteroaryl; R₂ is C₃-C₂₀ cycloalkyl, C₃-C₂₀ heterocycloalkyl, aryl, heteroaryl, ORₑ₁, or C₁-C₁₀ alkyl or C₁-C₁₀ heteroalkyl substituted with NRₑ₁Rₑ₂, N(Rₑ₁)—C(O)Rₑ₂, N(Rₑ₁)—C(O)Oₑ₂, N(Rₑ₁)—C(O)NRₑ₂Rₑ₃, N(Rₑ₁)—SO₂Rₑ₂, N(Rₑ₁)—C(S)NRₑ₂Rₑ₃, N(Rₑ₁)—C(NRₑ₂)—NRₑ₃Rₑ₄, or N(Rₑ₁)—C(NRₑ₂)—SRₑ₃; and each of R₃ and R₄, independently, is C₁-C₁₀ alkyl, C₃-C₂₀ cycloalkyl, C₃-C₂₀ heterocycloalkyl, aryl, heteroaryl, halo, cyano, amidino, guanidine, ureido, OR_{f1}, NR_{f1}R_{f2}, C(O)NR_{f1}R_{f2}, N(R_{f1})—C(O)R_{f2}, N(R_{f1})—C(O)OR_{f2}, C(O)R_{f1}, N(R_{f1})—C(S)NR_{f2}R_{f3}, N(R_{f1})—C(NR_{f2})—NR_{f3}R_{f4}, or N(R_{f1})—C(NR_{f2})—SR_{f3}; in which each of Rₐ₁, R_{b1}, R_{b2}, R_{c1}, R_{d1}, Rₑ₁, Rₑ₂, Rₑ₃, Rₑ₄, R_{f1}, R_{f2}, R_{f3}, and R_{f4}, independently, is H, C₁-C₁₀ alkyl, C₃-C₂₀ cycloalkyl, C₃-C₂₀ heterocycloalkyl, aryl, heteroaryl, cyano, OR, COOR, or C(O)NH₂; or R_{b1}, R_{b2}, and the carbon atom to which they are both attached, together are C₃-C₈ cycloalkyl or C₃-C₈ heterocycloalkyl; or R_{c1}, R₂, and the carbon atom to which they are both attached, together are C₃-C₈ cycloalkyl or C₃-C₈ heterocycloalkyl; R being H or C₁-C₁₀ alkyl. A subset of these pyrimidinone compounds are those in which A is phenyl or pyridyl; each of L₃ and L₄, independently, is —C(O)—, —CH₂—, —(CH₂)₂—, or —(CH₂)₃—; R₁ is phenyl substituted with F, OCH₃, or OCH₂CH₃; one of R₃ and R₄ is C₁-C₁₀ alkyl optionally substituted with phenyl, in which the phenyl is further substituted with F, Cl, or CF₃; and the other of R₃ and R₄ is C₃-C₂₀ heterocycloalkyl, heteroaryl, NR_{f1}R_{f2}, N(R_{f1})—C(O)R_{f1}, or N(R_{f1})—C(O)OR_{f2}.

In still another aspect, this invention features pyrimidinone compounds of formula (I) shown above in which A is aryl or heteroaryl; X is O, S, or NRₐ₁; L₁ is —C(R_{b1}R_{b2})—, C₂-C₁₀ alkylene, or C₂-C₁₀ heteroalkylene; L₂ is 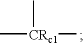
each of L₃ and L₄, independently, is —C(O)—, —SO₂—, —C(O)O—, —C(O)N_{d1}—, —C(O)CH₂—, —CH₂C(O)—, —SO₂CH₂—, —CH₂SO₂—, C₁-C₁₀ alkylene, or C₁-C₁₀ heteroalkylene; or L₃, L₄, and the nitrogen atom to which they are both attached, together are C₅-C₇ heterocycloalkyl or heteroaryl; or L₁, L₃, and the nitrogen atom to which they are both attached, together are C₅-C₇ heterocycloalkyl or heteroaryl; or L₁, L₄, and the nitrogen atom to which they are both attached, together are C₅-C₇ heterocycloalkyl or heteroaryl; R₁ is H, C₁-C₁₀ alkyl, C₃-C₂₀ cycloalkyl, C₃-C₂₀ heterocycloalkyl, aryl, or heteroaryl; R₂ is H, C₁-C₁₀ alkyl, C₃-C₂₀ cycloalkyl, C₃-C₂₀ heterocycloalkyl, aryl, heteroaryl, or L₂′-R₂′; L₂′ being —NRₑ₁—, —C(O)—, —SO₂—, —C(O)O—, —C(O)NRₑ₁—, —C(O)CH₂—, —CH₂C(O)—, —SO₂CH₂—, —CH₂SO₂—, C₁-C₁₀ alkylene, or C₁-C₁₀ heteroalkylene; R₂′ being H, C₁-C₁₀ alkyl, C₃-C₂₀ cycloalkyl, C₃-C₂₀ heterocycloalkyl, aryl, heteroaryl, halo, cyano, amidino, guanidine, ureido, ORₑ₂, NRₑ₂Rₑ₃, C(O)NRₑ₂Rₑ₃, N(Rₑ₂)—C(O)Rₑ₃, N(Rₑ₂)—C(O)ORₑ₃, C(O)Rₑ₂, N(Rₑ₂)—C(S)NRₑ₃Rₑ₄, N(R₁₂)—C(NRₑ₃)—NRₑ₄Rₑ₅, or N(Rₑ₂)—C(NRₑ₃)—SRₑ₄; and each of R₃ and R₄, independently, is C₁-C₁₀ alkyl, C₃-C₂₀ cycloalkyl, C₃-C₂₀ heterocycloalkyl, aryl, heteroaryl, halo, cyano, amidino, guanidine, ureido, OR_{f1}, NR_{f1}R_{f2}, C(O)NR_{f1}R_{f2}, N(R_{f1})—C(O)R_{f2}, N(R_{f1})—C(O)OR_{f2}, C(O)R_{f1}, N(R_{f1})—C(S)NR_{f2}R_{f3}, N(R_{f1})—C(NR_{f2})—NR_{f3}R_{f4}, or N(R_{f1})—C(NR_{f2})—SR_{f3}; in which each of Rₐ₁, R_{b1}, R_{b2}, R_{c1}, R_{d1}, Rₑ₁, Rₑ₂, Rₑ₃, Rₑ₄, Rₑ₅, R_{f1}, R_{f2}, R_{f3}, and R_{f4}, independently, is H, C₁-C₁₀ alkyl, C₃-C₂₀ cycloalkyl, C₃-C₂₀ heterocycloalkyl, aryl, heteroaryl, cyano, OR, COOR, or C(O)NH₂; or R_{b1}, R_{b2}, and the carbon atom to which they are both attached, together are C₃-C₈ cycloalkyl or C₃-C₈ heterocycloalkyl; or R_{c1}, R₂, and the carbon atom to which they are both attached, together are C₃-C₈ cycloalkyl or C₃-C₈ heterocycloalkyl; R being H or C₁-C₁₀ alkyl. A subset of these pyrimidinone compounds are those in which A is phenyl; each of L₃ and L₄, independently, is —C(O)—, —SO₂—, —CH₂—, —(CH₂)₂—, or —(CH₂)₃—; R₁ is phenyl substituted with OCH₃ or OCH₂CH₃, R₂ is H, NH₂, OCH₂CH₂N(CH₃)₂, or NHC(O)CH₂N(CH₃)₂; one of R₃ and R₄ is phenyl substituted with OCH₃ or methyl substituted with phenyl, in which the phenyl is further substituted with F, Cl, or CF₃; and the other of R₃ and R₄ is C₃-C₂₀ heterocycloalkyl, heteroaryl, NR_{f1}R_{f2}, C(O)NR_{f1}R_{f2}, N(R_{f1})—C(O)OR_{f2}, or N(R_{f1})—C(NR_{f2})—SR_{f3}.

In still another aspect, this invention features pyrimidinone compounds of formula (I) shown above in which A is aryl or heteroaryl; X is O, S, or NRₐ₁; L₁ is —C(R_{b1}R_{b2})—, C₂-C₁₀ alkylene, C₂-C₁₀ heteroalkylene, or deleted; L₂ is 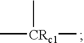
or L₂ and R₂ together are deleted; each of L₃ and L₄, independently, is —C(O)—, —SO₂—, —C(O)O—, —C(O)NR_{d1}—, —C(O)CH₂—, —CH₂C(O)—, —SO₂CH₂—, —CH₂SO₂—, C₁-C₁₀ alkylene, or C₁-C₁₀ heteroalkylene; or L₃, L₄, and the nitrogen atom to which they are attached, together are C₅-C₇ heterocycloalkyl or heteroaryl; or L₁, L₃, and the nitrogen atom to which they are both attached, together are C₅-C₇ heterocycloalkyl or heteroaryl; or L₁, L₄, and the nitrogen atom to which they are both attached, together are C₅-C₇ heterocycloalkyl or heteroaryl; R₁ is H, C₁-C₁₀ alkyl, C₃-C₂₀ cycloalkyl, C₃-C₂₀ heterocycloalkyl, aryl, or heteroaryl; R₂ is H, C₁-C₁₀ alkyl, C₃-C₂₀ cycloalkyl, C₃-C₂₀ heterocycloalkyl, aryl, heteroaryl, or ORₑ₁; or R₂ and L₂ together are deleted; and one of R₃ and R₄ is C₁-C₁₀ alkyl, C₃-C₂₀ cycloalkyl, C₃-C₂₀ heterocycloalkyl, aryl, heteroaryl, halo, cyano, amidino, guanidine, ureido, OR_{f1}, NR_{f1}R_{f2}, C(O)NR_{f1}R_{f2}, N(R_{f1})—C(O)R_{f2}, N(R_{f1})—C(O)OR_{f2}, C(O)R_{f1}, N(R_{f1})—C(S)NR_{f2}R_{f3}, N(R_{f1})—C(NR_{f2})—NR_{f1}R_{f4}, or N(R_{f1})—C(NR_{f2})—SR_{f3}; the other of R₃ and R₄ is N(R_{f1})—C(NR_{f2})—SR_{f3}; in which each of Rₐ₁, R_{b1}, R_{b2}, R_{c1}, R_{d1}, Rₑ₁, R_{f1}, R_{f2}, R_{f3}, and R_{f4}, independently, is H, C₁-C₁₀ alkyl, C₃-C₂₀ cycloalkyl, C₃-C₂₀ heterocycloalkyl, aryl, heteroaryl, cyano, OR, COOR, or C(O)NH₂; or R_{b1}, R_{b2}, and the carbon atom to which they are both attached, together are C₃-C₈ cycloalkyl or C₃-C₈ heterocycloalkyl; or R_{c1}, R₂, and the carbon atom to which they are both attached, together are C₃-C₈ cycloalkyl or C₃-C₈ heterocycloalkyl; R being H or C₁-C₁₀ alkyl. A subset of these pyrimidinone compounds are those in which A is phenyl; each of L₃ and L₄, independently, is —C(O)— or —(CH₂)₂—; R₁ is phenyl substituted with OCH₃ or OCH₂CH₃; R₂ is methyl; one of R₃ and R₄ is methyl substituted with chloro-substituted phenyl.

In still another aspect, this invention features pyrimidinone compounds of formula (I) shown above in which A is aryl or heteroaryl; X is O, S, or NRₐ₁; L₁ is deleted; L₂ and R₂ together are deleted; each of L₃ and L₄, independently, is —C(O)—, —SO₂—, —C(O)O—, —C(O)NR_{b1}—, —C(O)CH₂—, —CH₂C(O)—, —SO₂CH₂—, —CH₂SO₂—, C₁-C₁₀ alkylene, or C₁-C₁₀ heteroalkylene; or L₃, L₄, and the nitrogen atom to which they are attached, together are C₅-C₇ heterocycloalkyl or heteroaryl; R₁ is H, C₁-C₁₀ alkyl, C₃-C₂₀ cycloalkyl, C₃-C₂₀ heterocycloalkyl, aryl, or heteroaryl; and each of R₃ and R₄, independently, is C₁-C₁₀ alkyl, C₃-C₂₀ cycloalkyl, C₃-C₂₀ heterocycloalkyl, aryl, heteroaryl, halo, cyano, amidino, guanidine, ureido, OR_{c1}, NR_{c1}R_{c2}, C(O)NR_{c1}R_{c2}, N(R_{c1})—C(O)R_{c2}, N(R_{c1})—C(O)OR_{c}, C(O)R_{c1}, N(R_{c1})—C(S)NR_{c2}R_{c3}, N(R_{c1})—C(NR_{c2})—NR_{c3}R_{c4}, or N(R_{c1})—C(NR_{c2})—SR_{c3}; in which each of Rₐ₁, R_{b1}, R_{c1}, R_{c2}, R_{c3}, and R_{c4}, independently, is H, C₁-C₁₀ alkyl, C₃-C₂₀ cycloalkyl, C₃-C₂₀ heterocycloalkyl, aryl, heteroaryl, cyano, OR, COOR, or C(O)NH₂; R being H or C₁-C₁₀ alkyl. A subset of these pyrimidinone compounds are those in which A is phenyl; each of L₃ and L₄, independently, is —C(O)— or —(CH₂)₂—; one of R₃ and R₄ is phenyl substituted with CF₃; and the other of R₃ and R₄ is C₃-C₂₀ heterocycloalkyl.

In still another aspect, this invention features pyrimidinone compounds of formula (II):

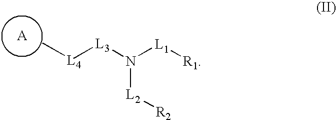

In this formula, A is heteroaryl; each of L₁ and L₂, independently, is —C(O)—, —SO₂—, —C(O)O—, —C(O)NRₐ₁—, —C(O)CH₂—, —CH₂C(O)—, —SO₂CH₂—, —CH₂SO₂—, C₁-C₁₀ alkylene, or C₁-C₁₀ heteroalkylene; and each of L₃ and L₄, independently, is —C(R_{b1}R_{b2})—, C₂-C₁₀ alkylene, C₂-C₁₀ heteroalkylene, or deleted; or L₁, L₂, and the nitrogen atom to which they are both attached, together are C₅-C₇ heterocycloalkyl or heteroaryl; or L₁, L₃, and the nitrogen atom to which they are both attached, together are C₅-C₇ heterocycloalkyl or heteroaryl; or L₂, L₃, and the nitrogen atom to which they are both attached, together are C₅-C₇ heterocycloalkyl or heteroaryl; each of R₁ and R₂, independently, is C₁-C₁₀ alkyl, C₃-C₂₀ cycloalkyl, C₃-C₂₀ heterocycloalkyl, aryl, heteroaryl, halo, cyano, amidino, guanidine, ureido, OR_{c1}, NR_{c1}R_{c2}, C(O)N_{c1}R_{f2}, N(R_{c1})—C(O)R_{c2}, N(R_{c1})—C(O)OR_{c2}, C(O)R_{c1}, N(R_{c1})—C(S)NR_{c2}R_{c3}, N(R_{c1})—C(NR_{c2})—NR_{c3}R_{c4}, or N(R_{c1})—C(NR_{c2})—SR_{c3}; in which each of Rₐ₁, R_{c1}, R_{c2}, R_{c3}, and R_{c4}, independently, is H, C₁-C₁₀ alkyl, C₃-C₂₀ cycloalkyl, C₃-C₂₀ heterocycloalkyl, aryl, heteroaryl; and each of R_{b1} and R_{b2}, independently, is H, C₁-C₁₀ alkyl, C₃-C₂₀ cycloalkyl, C₃-C₂₀ heterocycloalkyl, aryl, heteroaryl, or L-R; L being —C(O)—, —SO₂—, —C(O)O—, —C(O)NR₁—, —C(O)CH₂—, —CH₂C(O)—, —SO₂CH₂—, —CH₂SO₂—, C₁-C₁₀ alkylene, or C₁-C₁₀ heteroalkylene; R being C₁-C₁₀ alkyl, C₃-C₂₀ cycloalkyl, C₃-C₂₀ heterocycloalkyl, aryl, heteroaryl, halo, cyano, amidino, guanidine, ureido, OR₂, NR₂R₃, C(O)NR₂R₃, N(R₂)—C(O)R₃, N(R₂)—C(O)OR₃, C(O)R₂, N(R₂)—C(S)NR₃R₄, N(R₂)—C(NR₃)—NR₄R₅, or N(R₂)—C(NR₃)—SR₄; or R_{b1}, R_{b2}, and the carbon atom to which they are both attached, together are C₃-C₈ cycloalkyl or C₃-C₈ heterocycloalkyl; each of R₁, R₂, R₃, R₄, and R₅, independently, being H or C₁-C₁₀ alkyl.

In still another aspect, this invention features a method for treating an inflammatory or immune disease. The method includes administering to a subject in need of treatment of an effective amount of one or more pyrimidinone compounds of formula (I) shown above. “Treatment” refers to administering one or more pyrimidinone compounds to a subject, who has an inflammatory or immune disease, a symptom of such a disease, or a predisposition toward such a disease, with the purpose to confer a therapeutic effect, e.g., to cure, relieve, alter, affect, ameliorate, or prevent the inflammatory or immune disease, the symptom of it, or the predisposition toward it. “An effective amount” refers to the amount of one or more active pyrimidinone compounds that is required to confer a therapeutic effect on a treated subject.

An inflammatory disease is characterized by a local or systemic, acute or chronic inflammation. An immune disease is characterized by a hyper- or hypo-reaction of the immune system. Examples of inflammatory or immune diseases include neurodegenerative diseases (e.g., Alzheimer's disease), multiple sclerosis, systemic lupus erythematosus, rheumatoid arthritis, ankylosing spondylitis, psoriatic arthritis, juvenile rheumatoid arthritis, atherosclerosis, vasculitis, chronic heart failure, cerebrovascular ischemia, encephalitis, meningitis, hepatitis, nephritis, sepsis, sarcoidosis, psoriasis, eczema, uticaria, type I diabetes, asthma, conjunctivitis, otitis, allergic rhinitis, chronic obstructive pulmonary disease, sinusitis, dermatitis, inflammatory bowel disease, ulcerative colitis, Crohn's disease, Behcet's syndrome, pulmonary fibrosis, endometriosis, gout, cancer, cachexia, viral infections, bacterial infections, organ transplant conditions, skin transplant conditions, and graft versus host diseases.

A subject in need of treatment of an inflammatory or immune disease can also be concurrently administered with a pyrimidinone compound described above and one or more other therapeutic agents at the same time or at different times during the period of treatment. Examples of such a therapeutic agent include glucocorticoids (e.g., predinisolone), NSAIDs (e.g., acetaminophene), COX-2 inhibitors (e.g., celebrex), TNF-α inhibitors (e.g., embrel), immunosuppressive agents (e.g., cyclosporin A), tarcolimus (e.g., FK506), and methotrexate.

In a further aspect, this invention features a pharmaceutical composition that contains an effective amount of at least one of the above-mentioned pyrimidinone compounds and a pharmaceutically acceptable carrier. The pharmaceutical composition may further contain a second therapeutic agent as described above.

The pyrimidinone compounds described above include the compounds themselves, as well as their salts, prodrugs, and solvates, if applicable. A salt, for example, can be formed between an anion and a positively charged group (e.g., amino) on a pyrimidinone compound. Suitable anions include chloride, bromide, iodide, sulfate, bisulfate, sulfamate, nitrate, phosphate, citrate, methanesulfonate, trifluoroacetate, maleate, succinate, fumarate, tartrate, salicylate, lactate, naphthalenesulfonate, and acetate. Likewise, a salt can also be formed between a cation and a negatively charged group (e.g., carboxylate) on a pyrimidinone compound. Suitable cations include sodium ion, potassium ion, magnesium ion, calcium ion, and an ammonium cation such as tetramethylammonium ion. The pyrimidinone compounds also include those salts containing quaternary nitrogen atoms. Examples of prodrugs include esters and other pharmaceutically acceptable derivatives, which, upon administration to a subject, are capable of providing active pyrimidinone compounds. A solvate refers to a complex formed between an active pyrimidinone compound described above and a pharmaceutically acceptable solvent. Examples of pharmaceutically acceptable solvents include water, ethanol, isopropanol, ethyl acetate, acetic acid, and ethanolamine.

Also within the scope of this invention is a composition containing one or more of the pyrimidinone compounds described above for use in treating an inflammatory disease or an immune disease, and the use of such a composition for the manufacture of a medicament for the just-mentioned treatment.

The details of one or more embodiments of the invention are set forth in the description below. Other features, objects, and advantages of the invention will be apparent from the description and from the claims.

### DETAILED DESCRIPTION

Shown below are exemplary compounds of this invention.

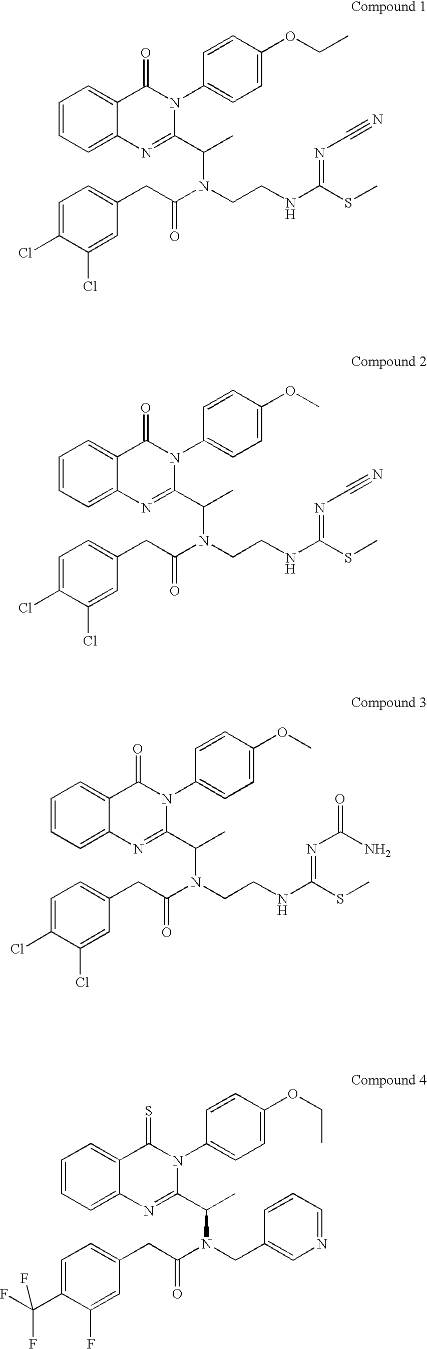

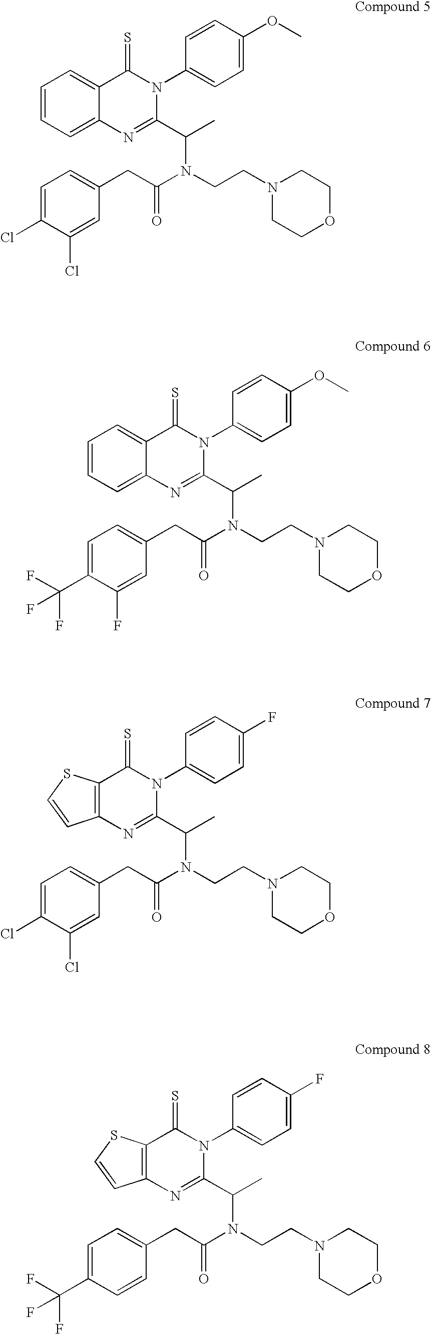

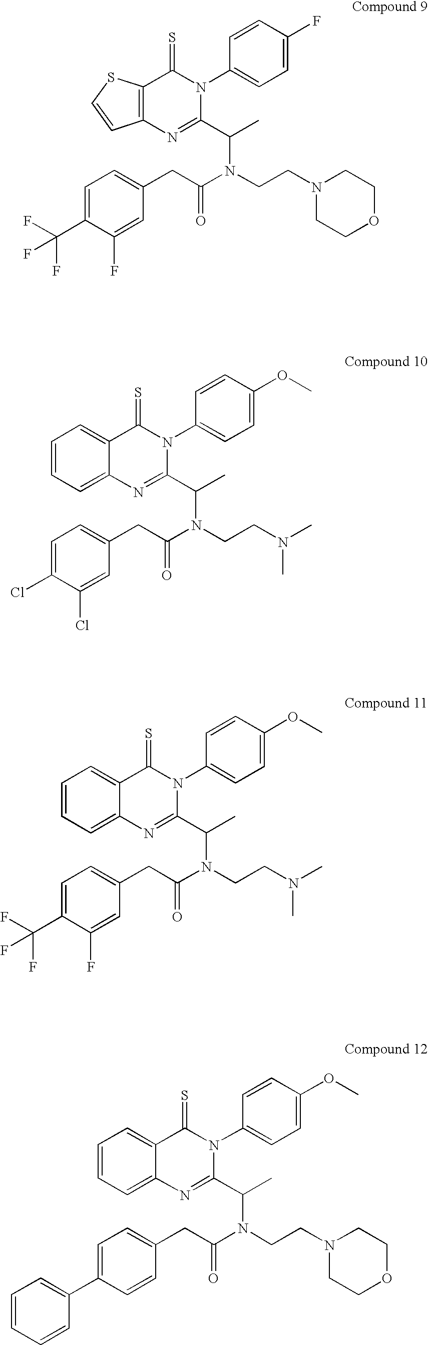

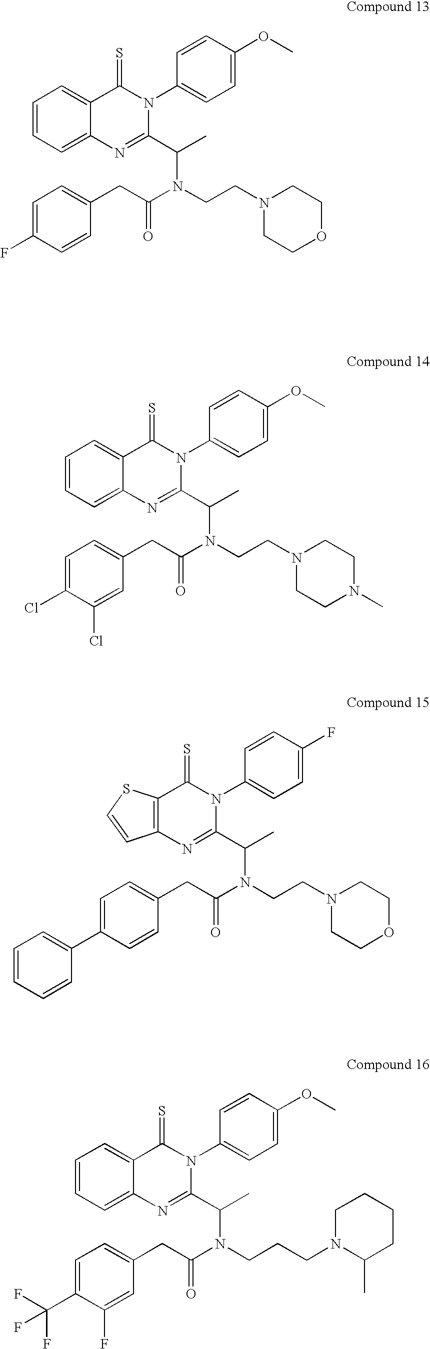

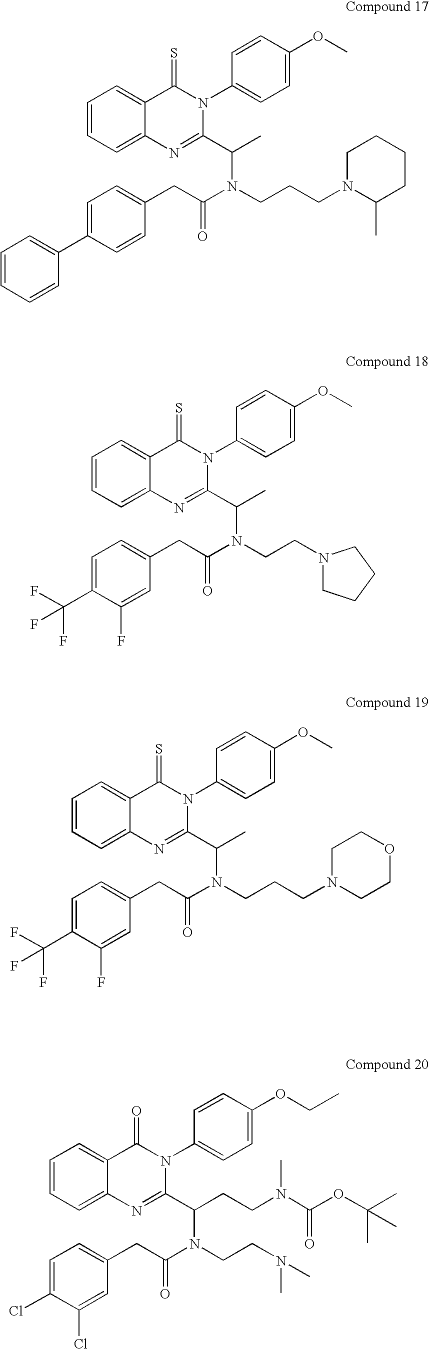

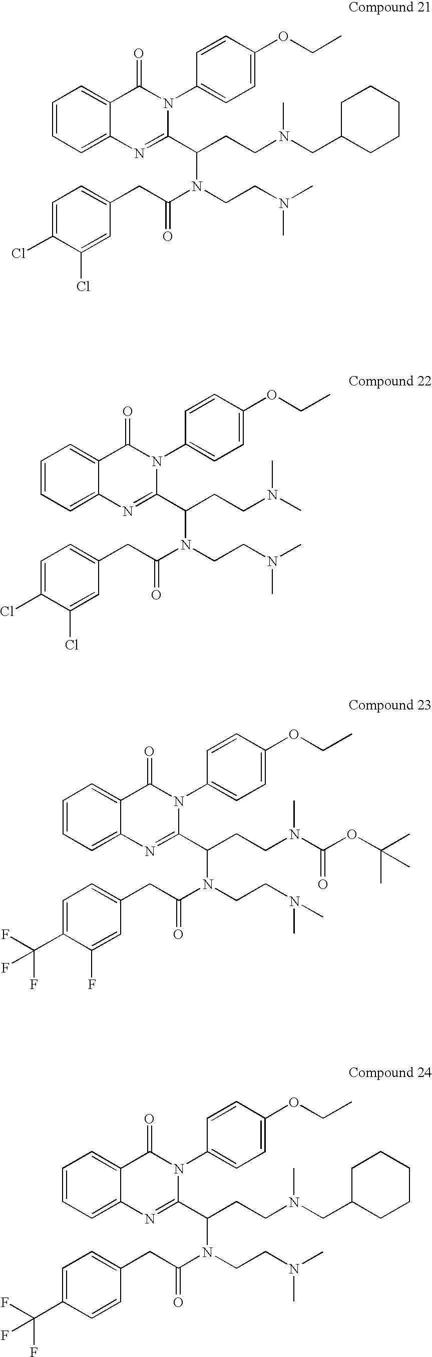

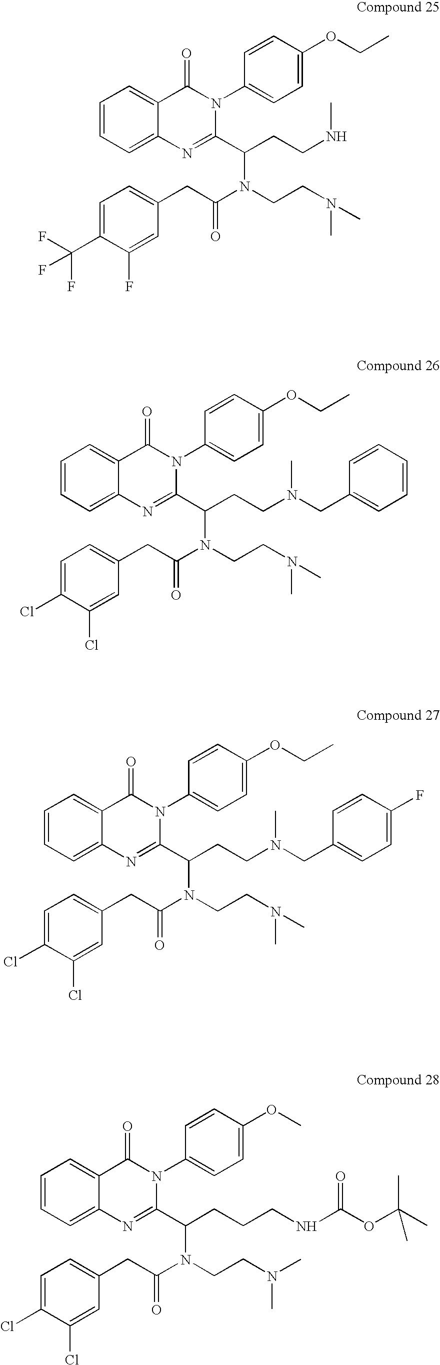

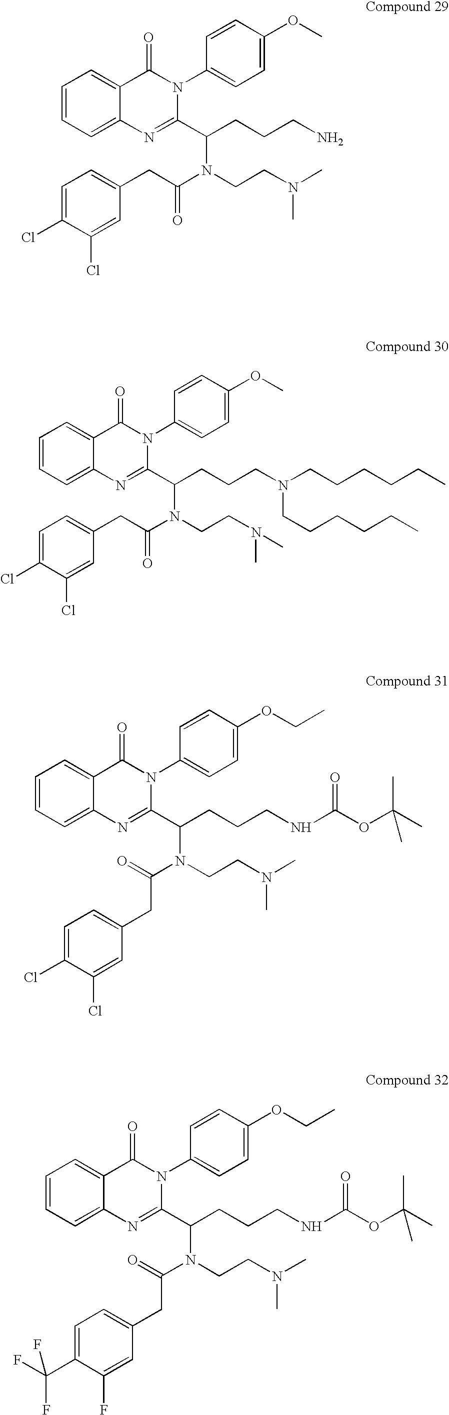

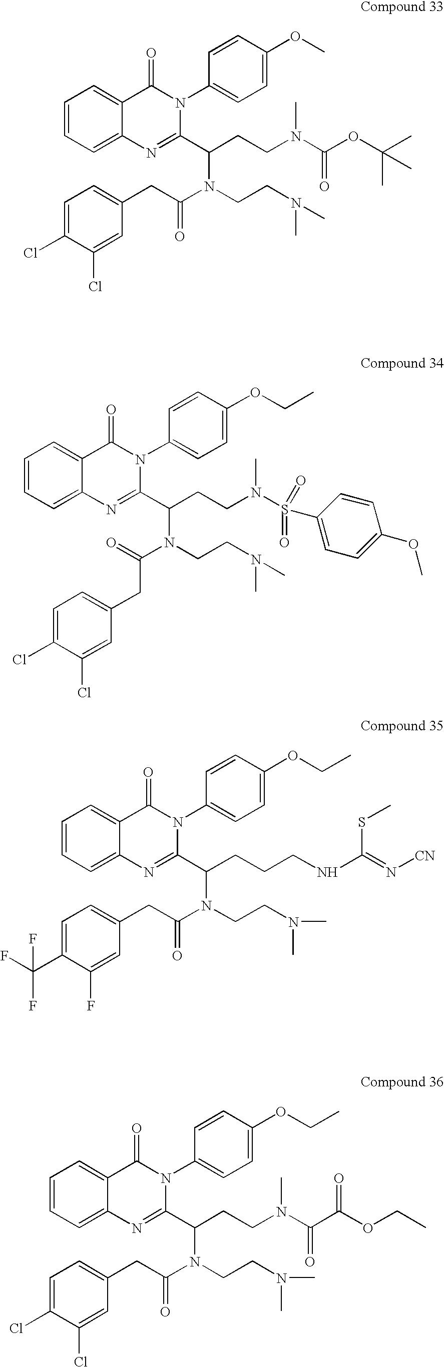

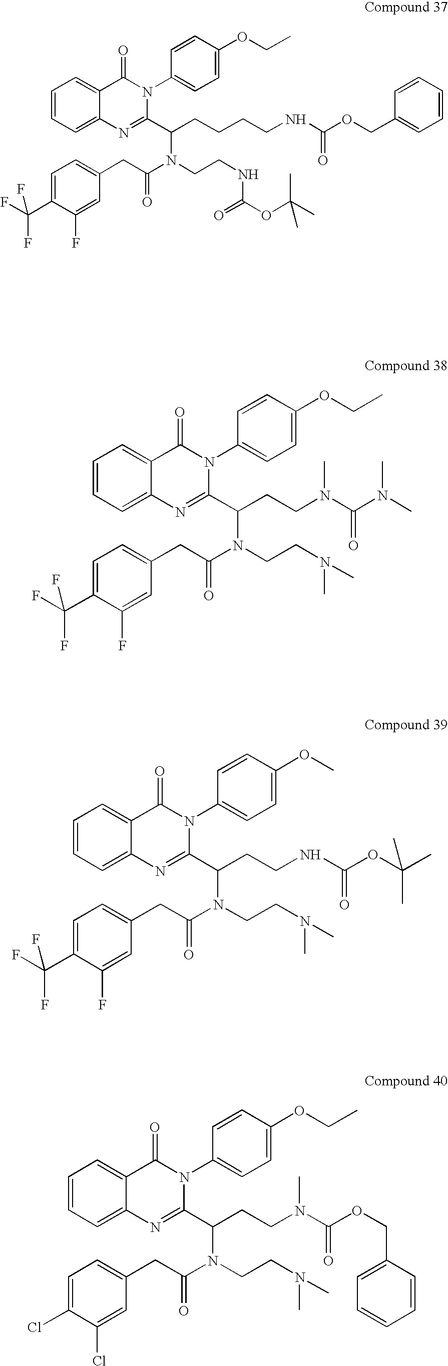

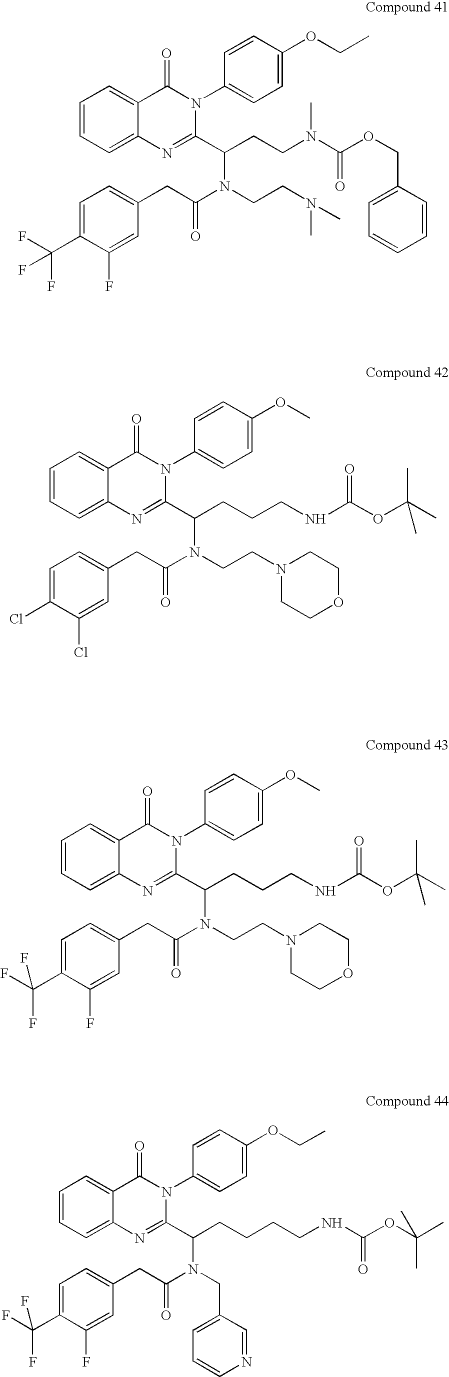

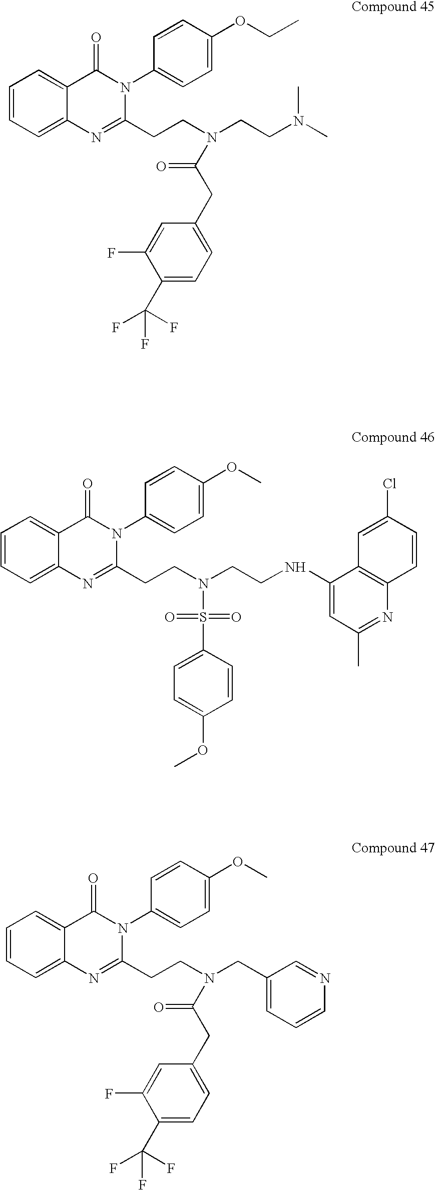

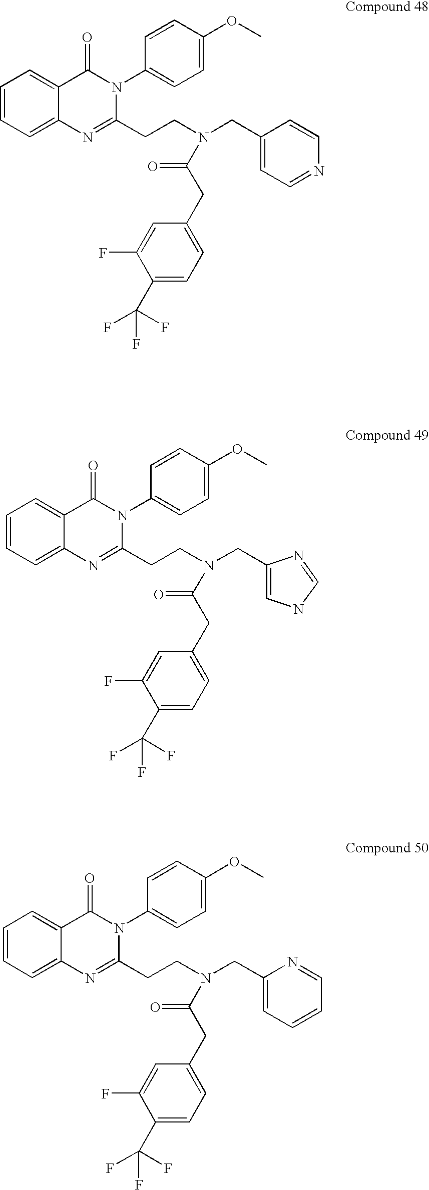

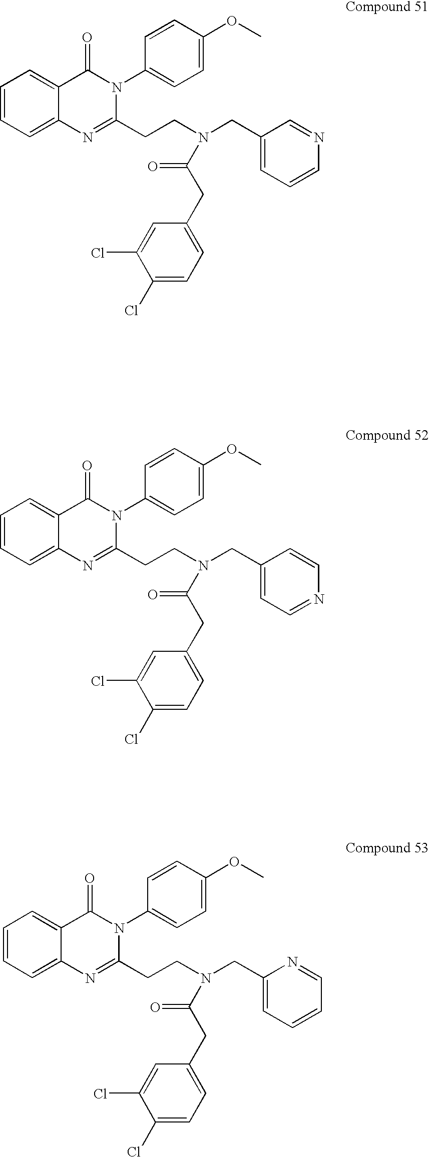

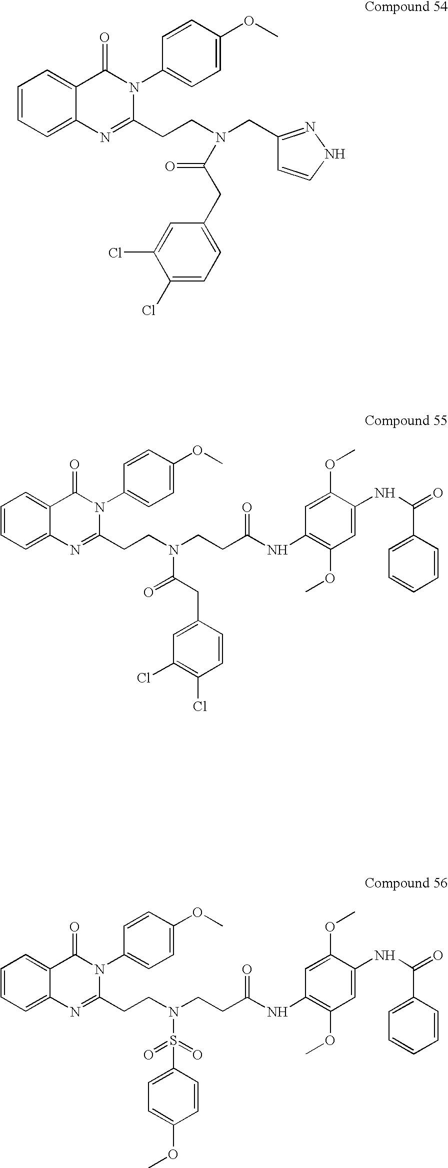

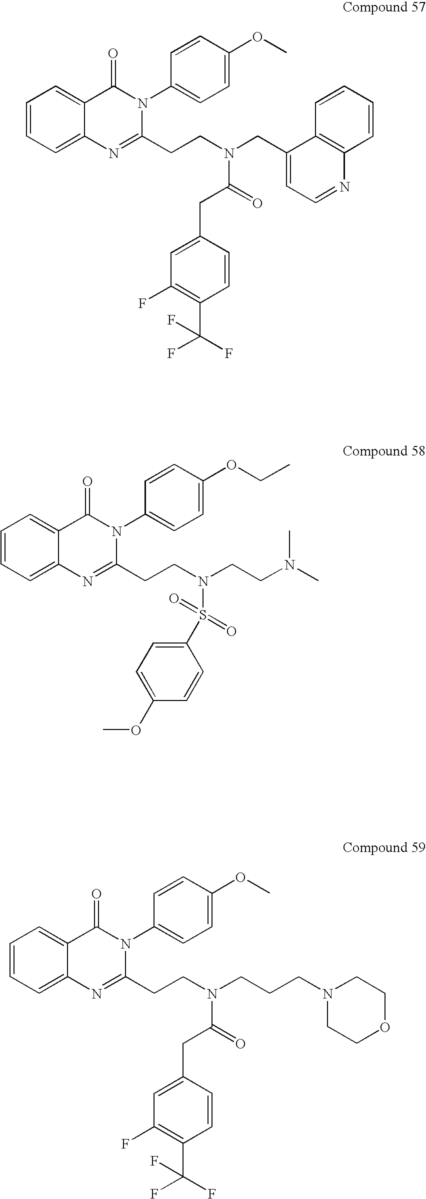

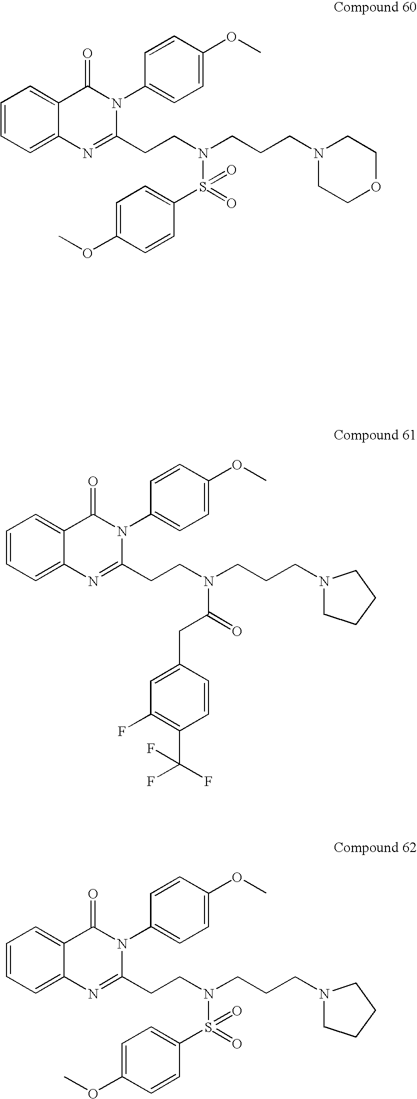

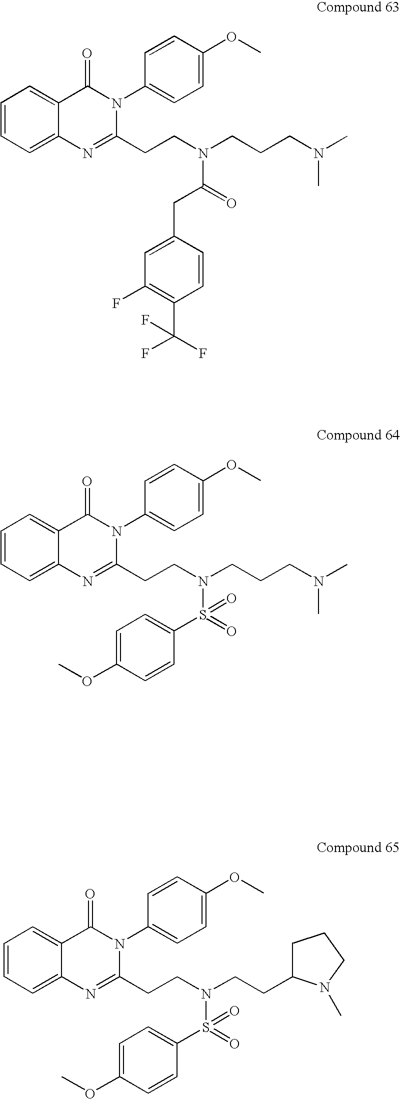

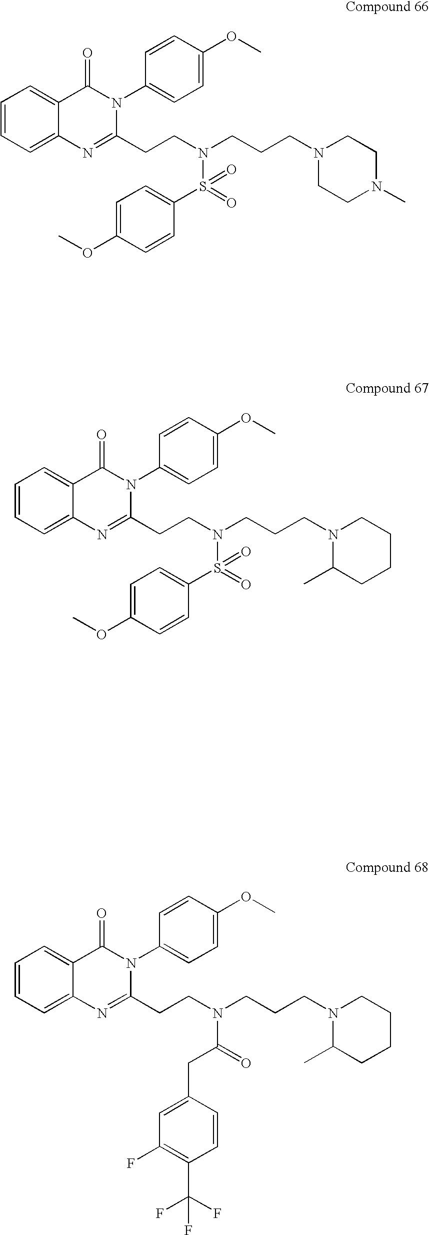

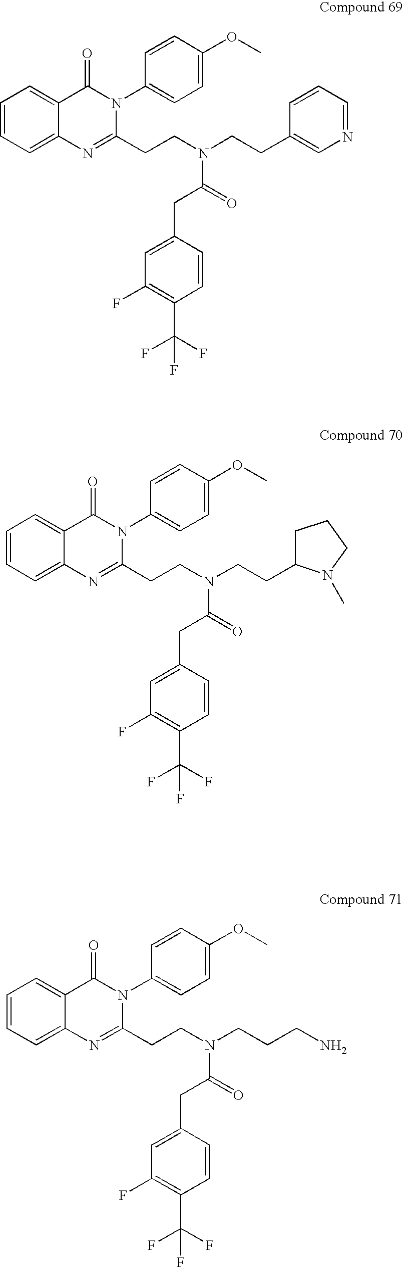

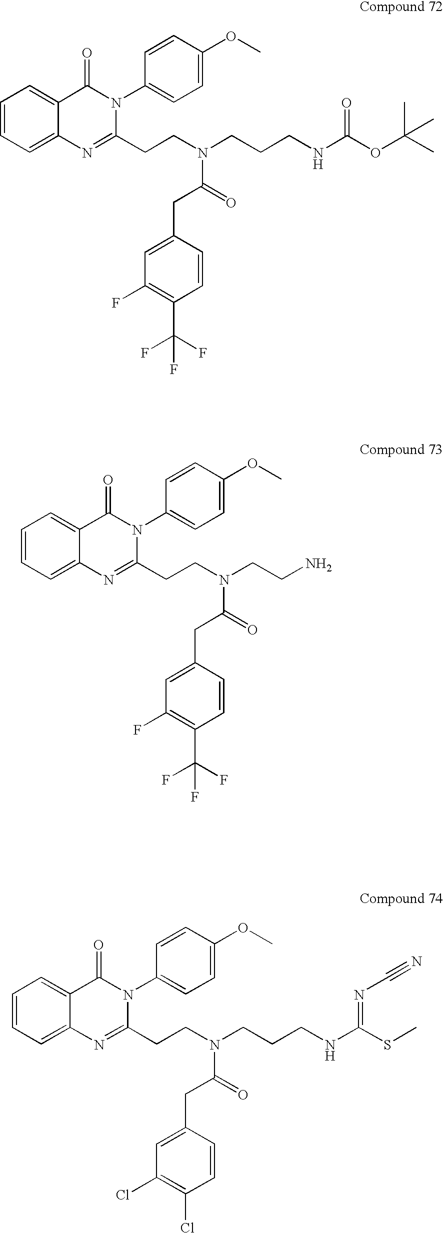

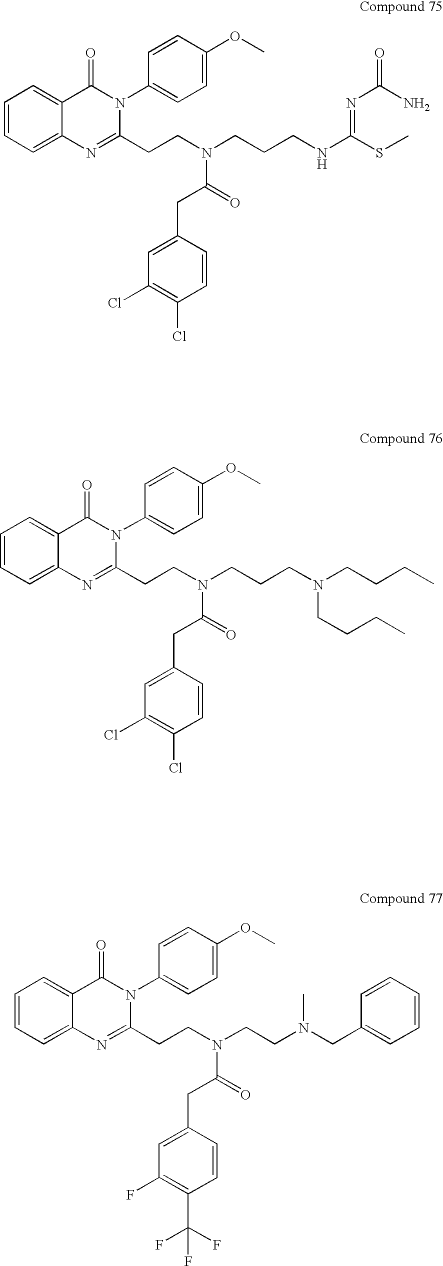

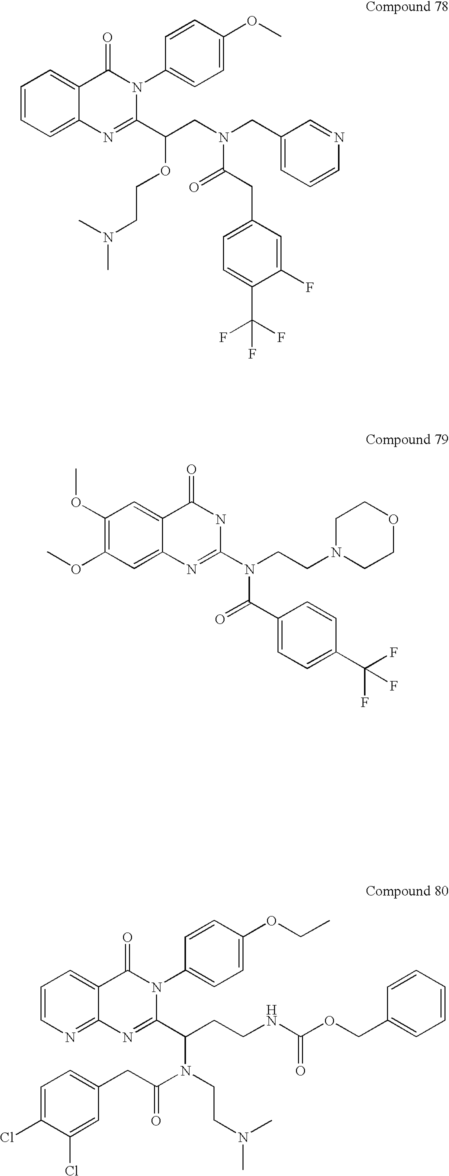

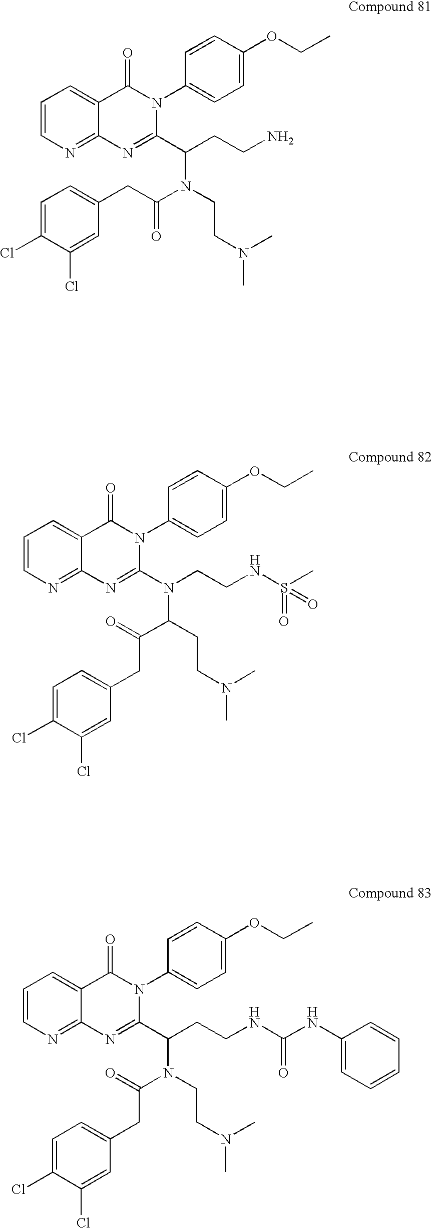

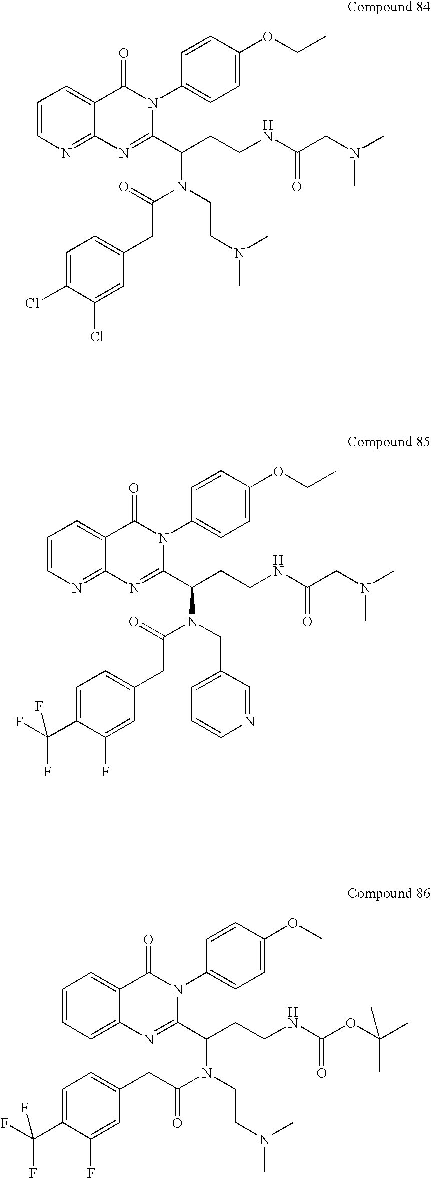

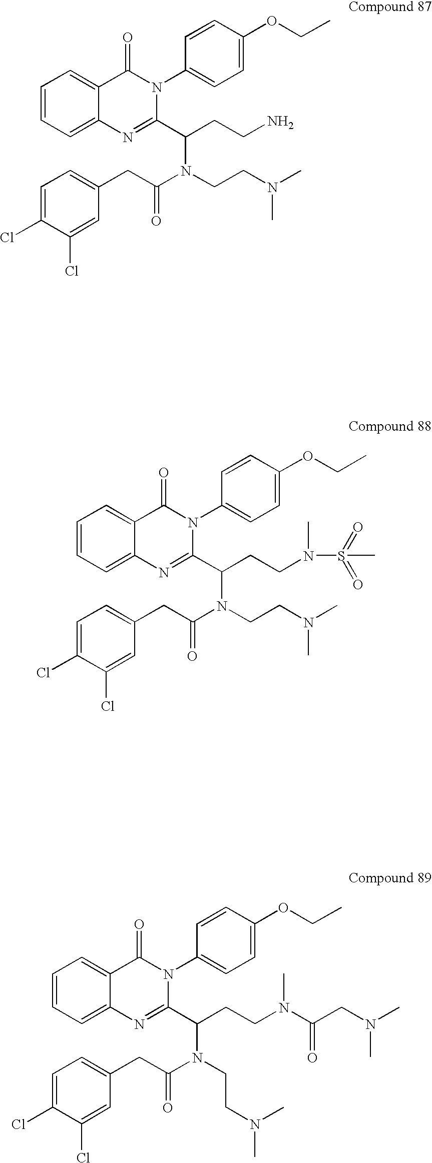

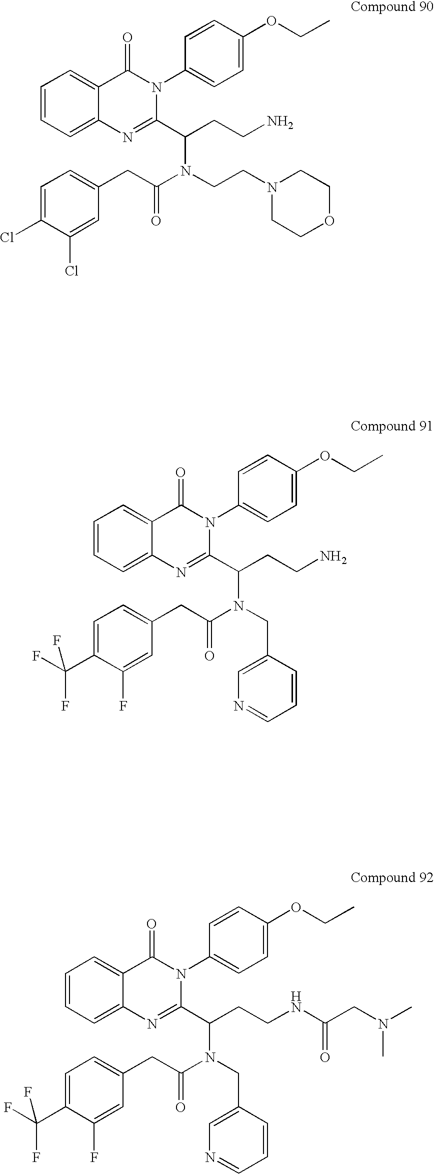

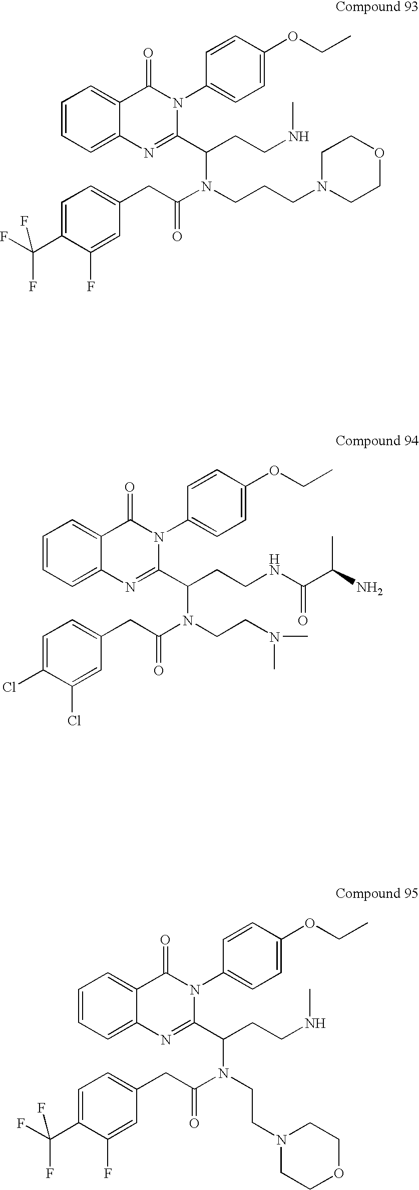

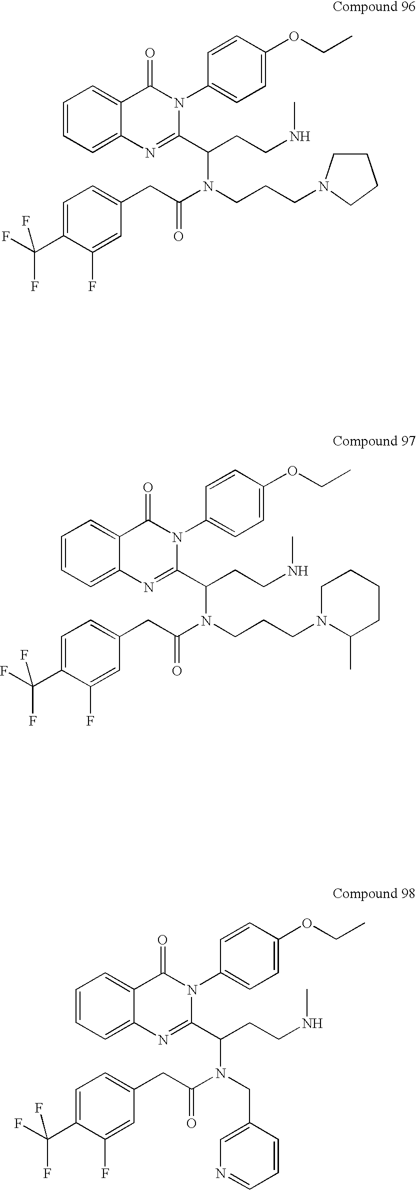

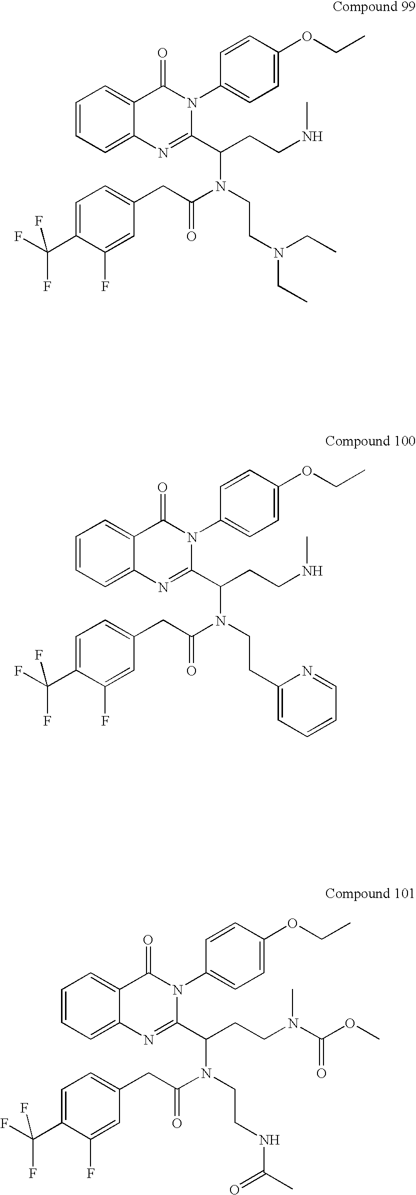

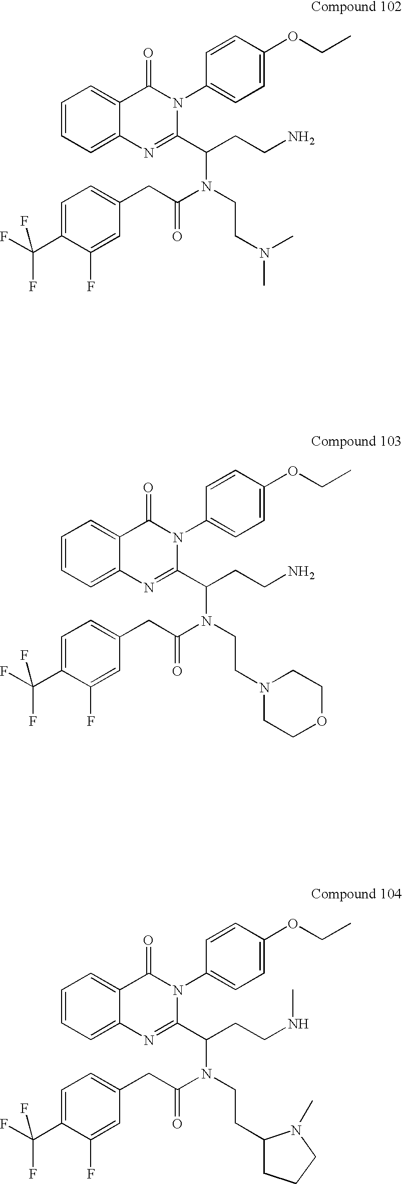

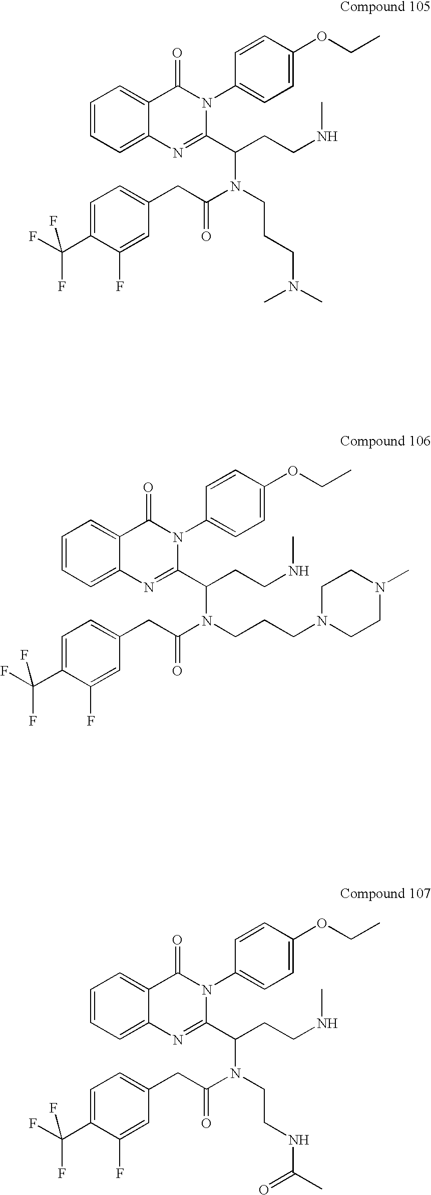

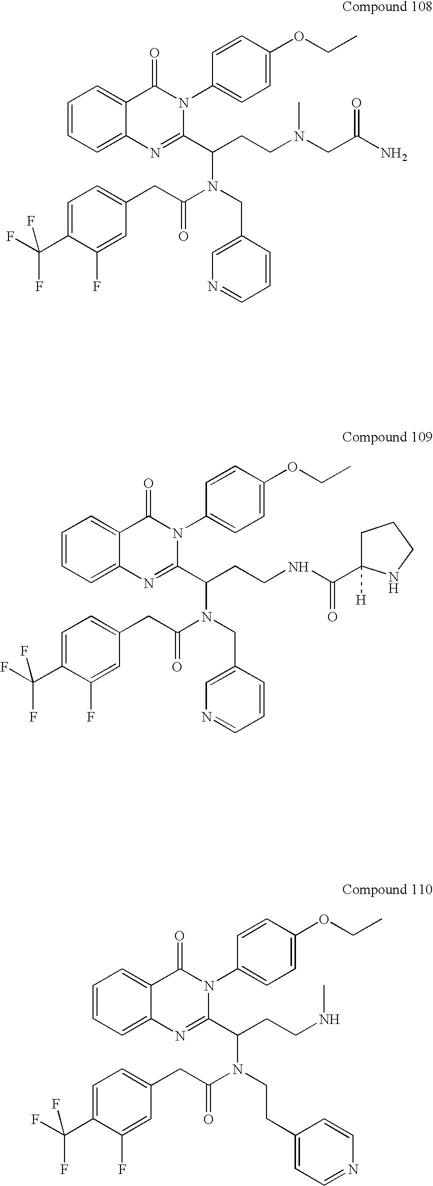

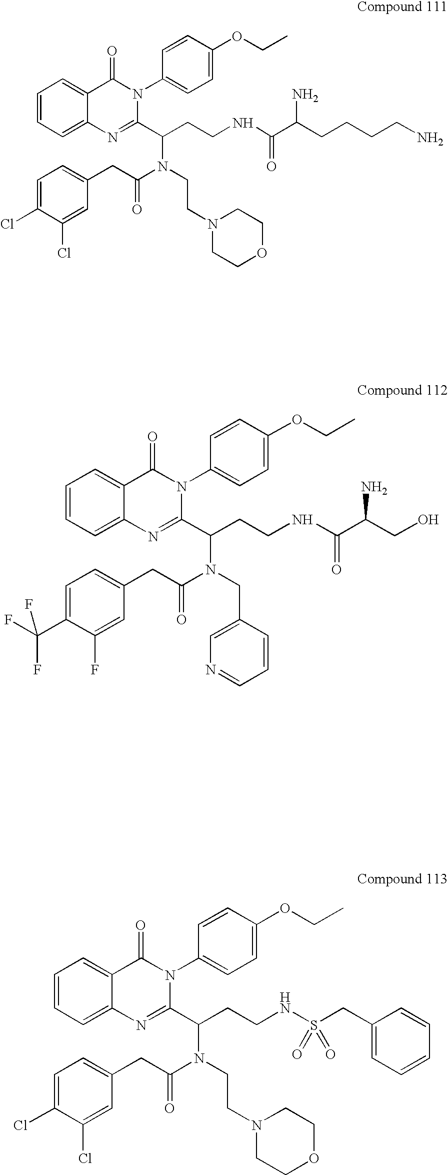

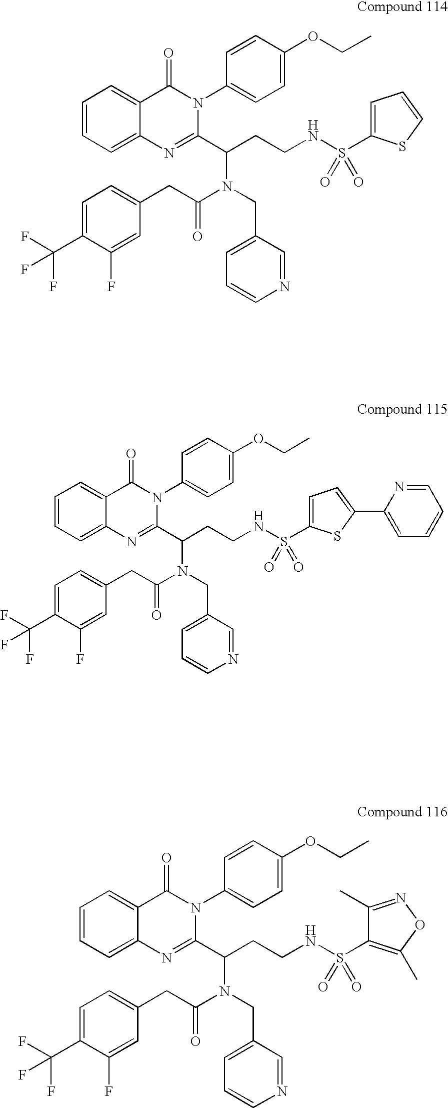

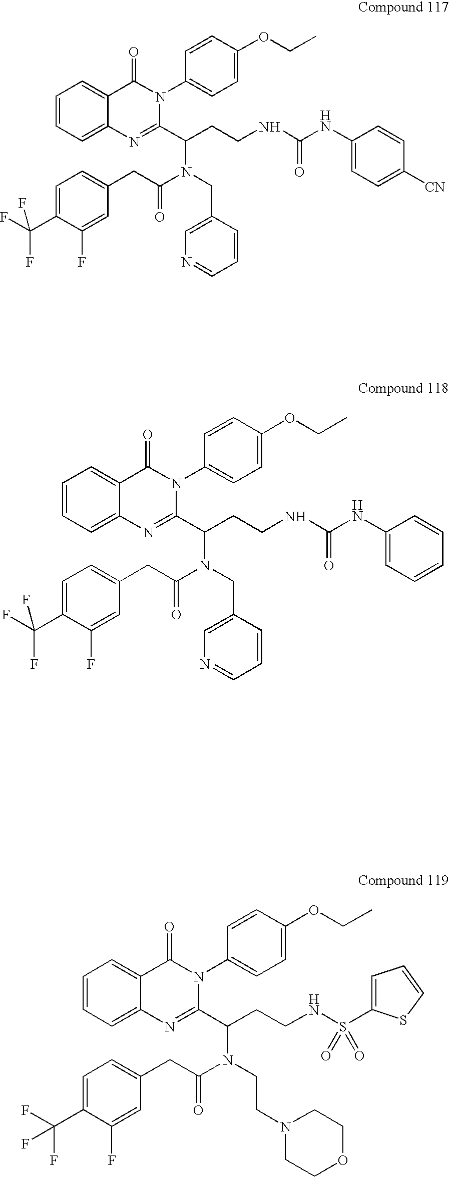

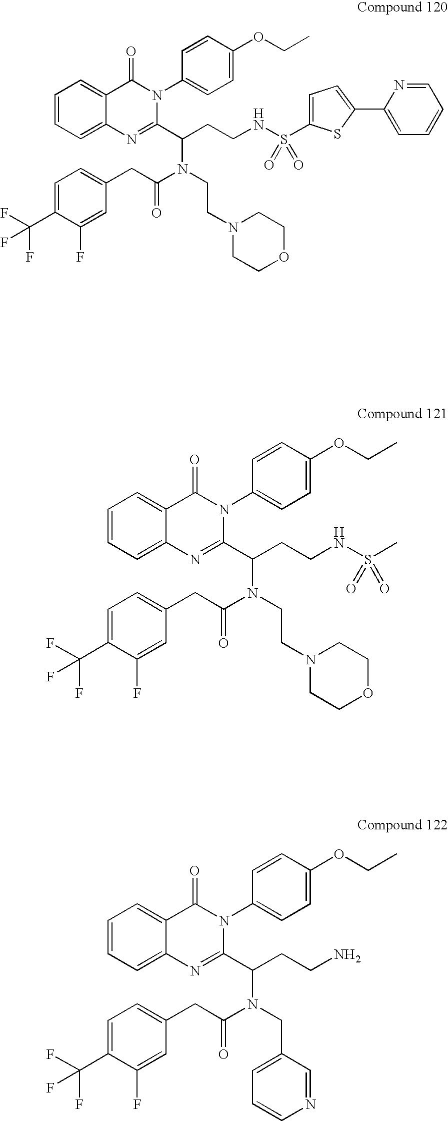

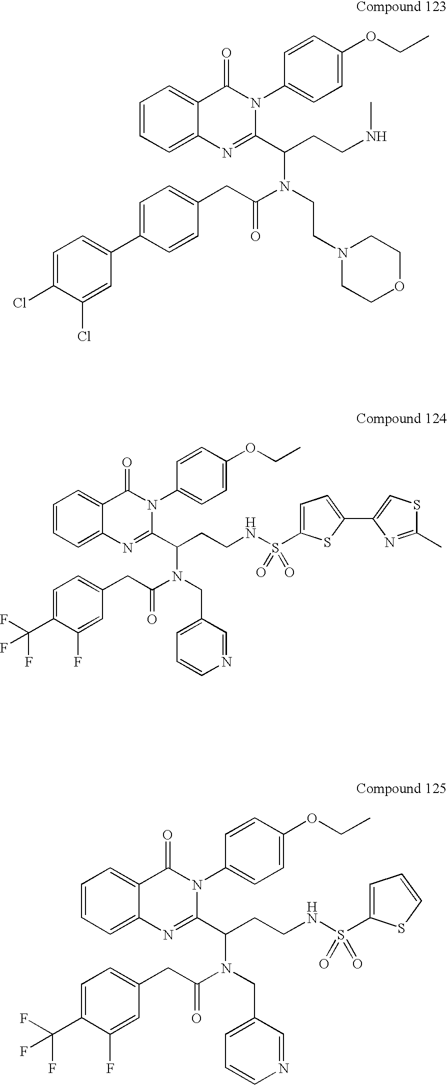

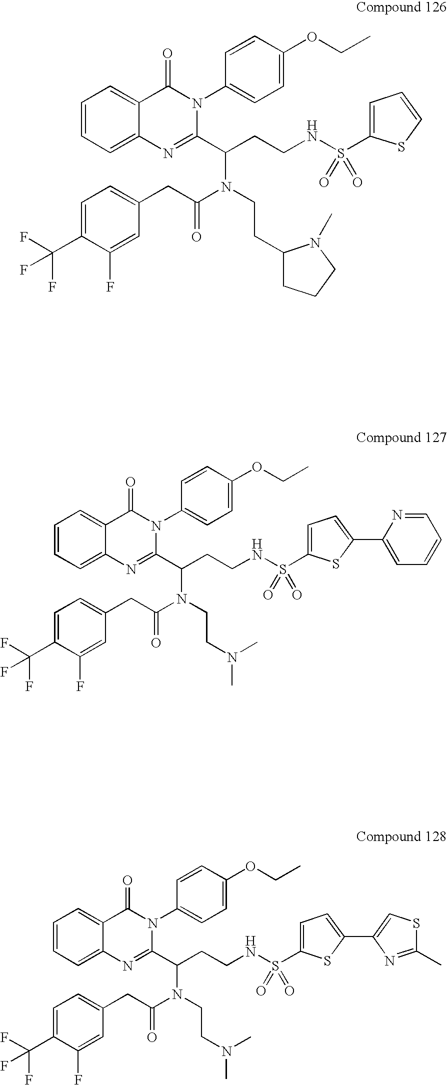

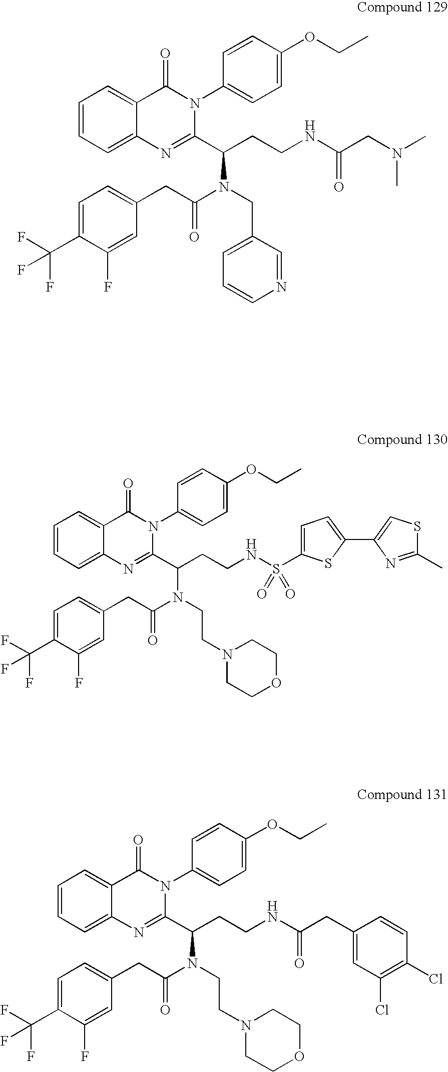

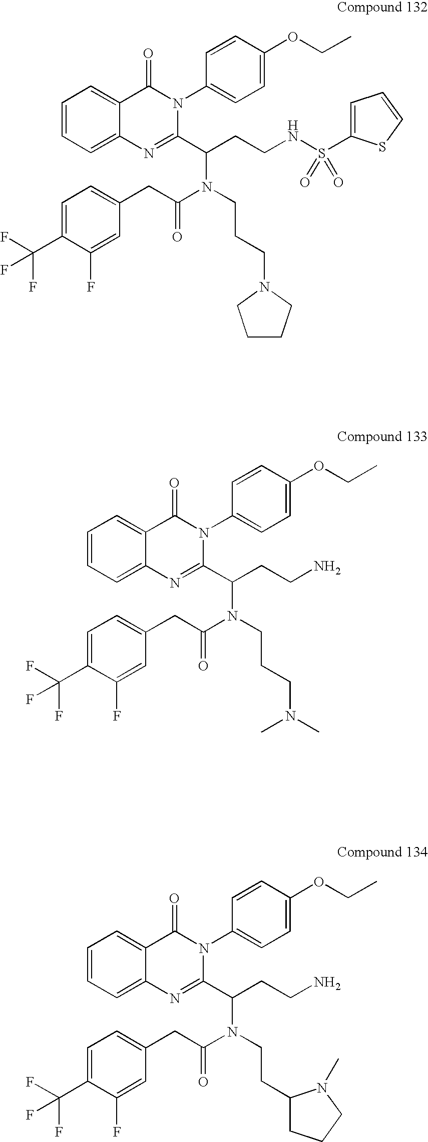

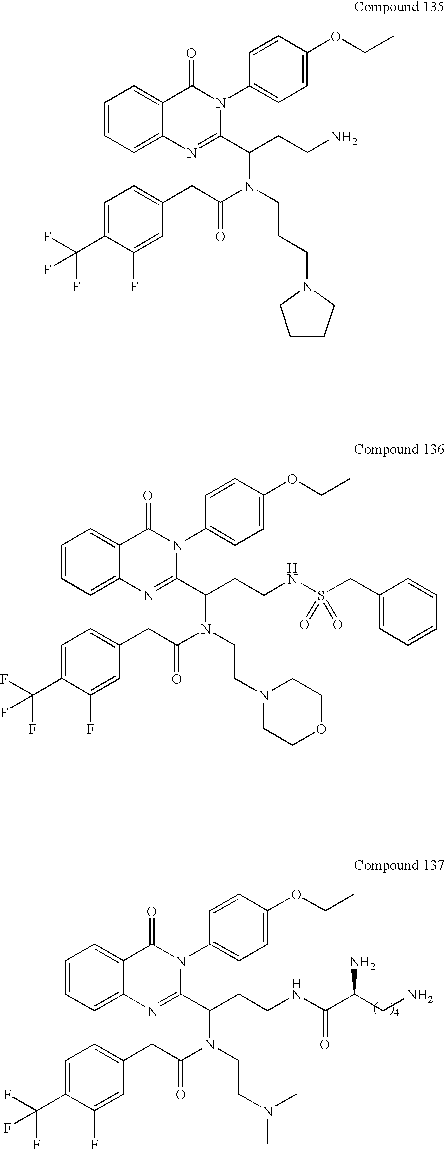

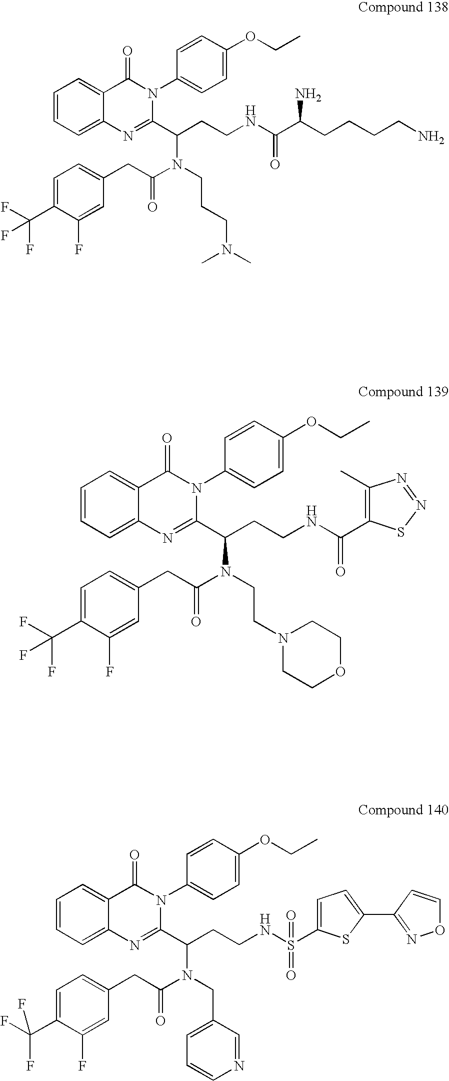

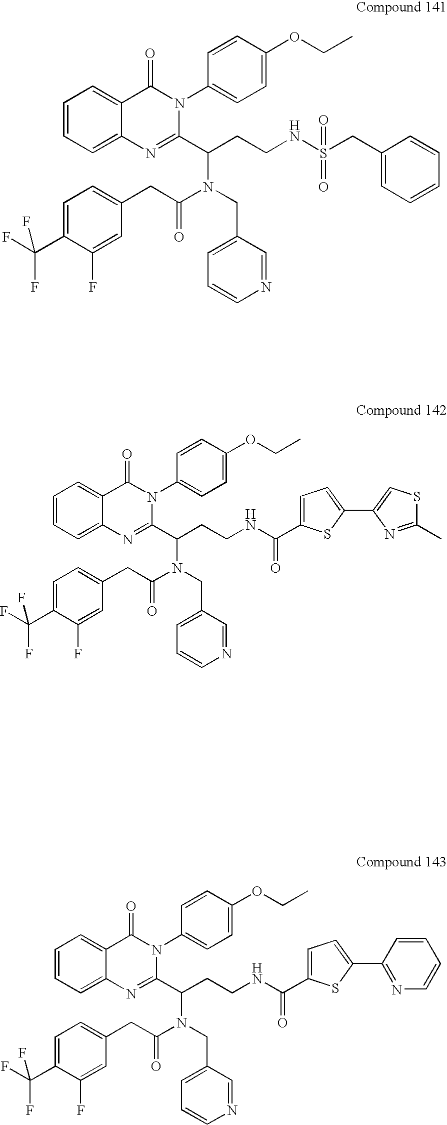

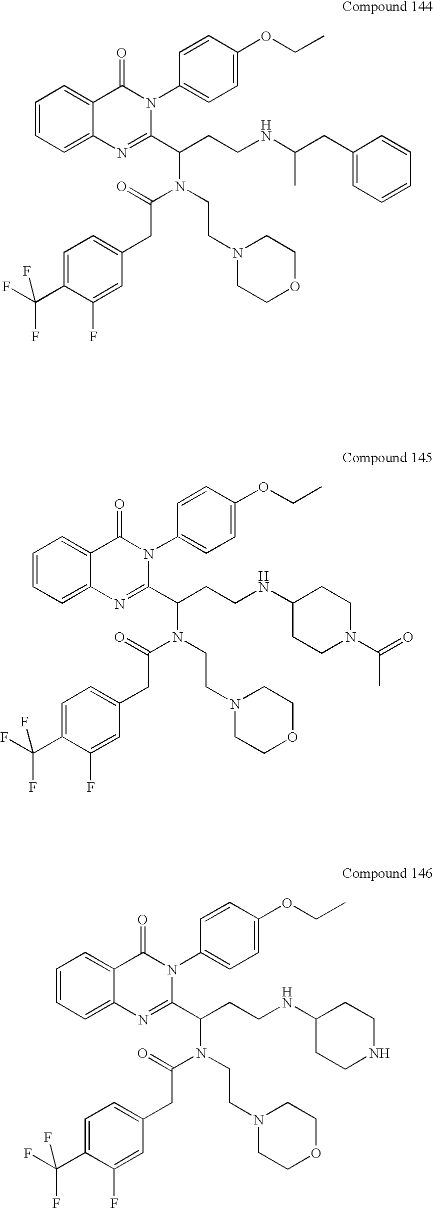

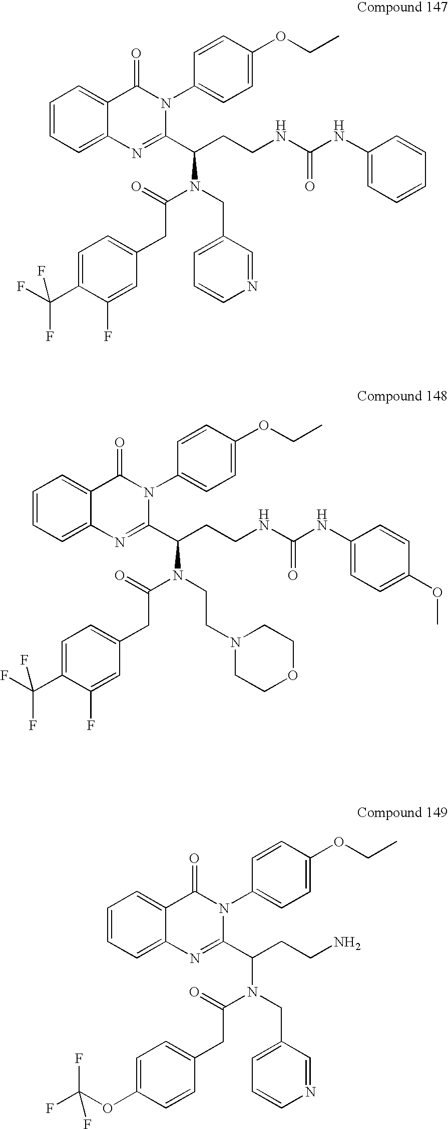

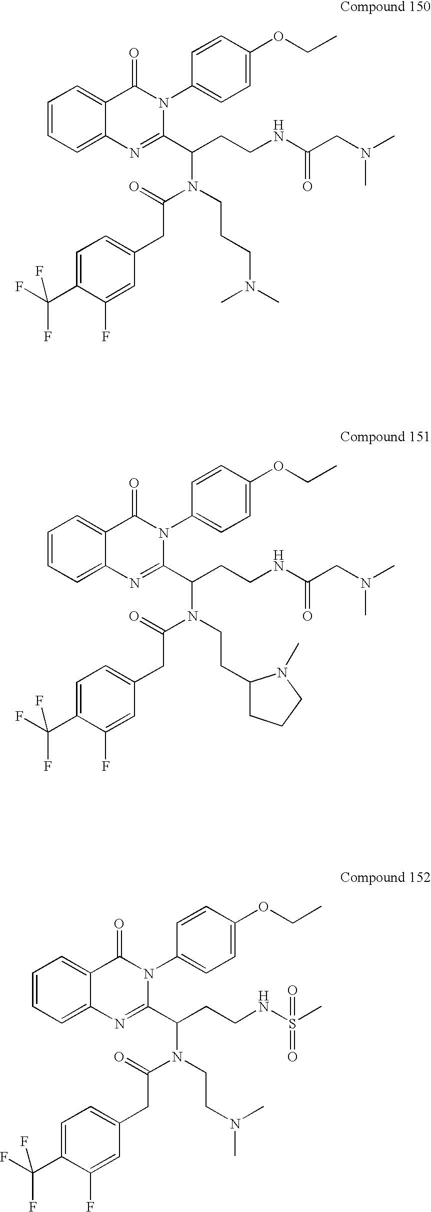

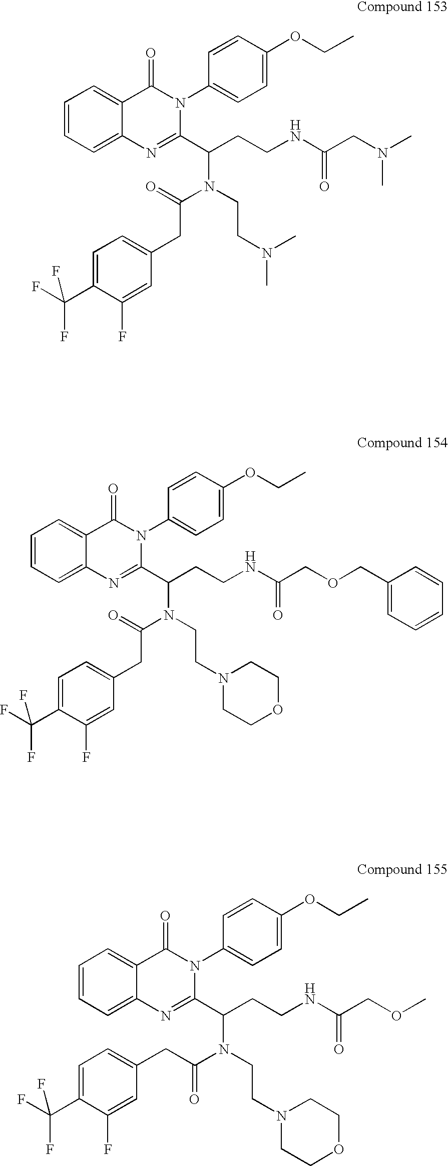

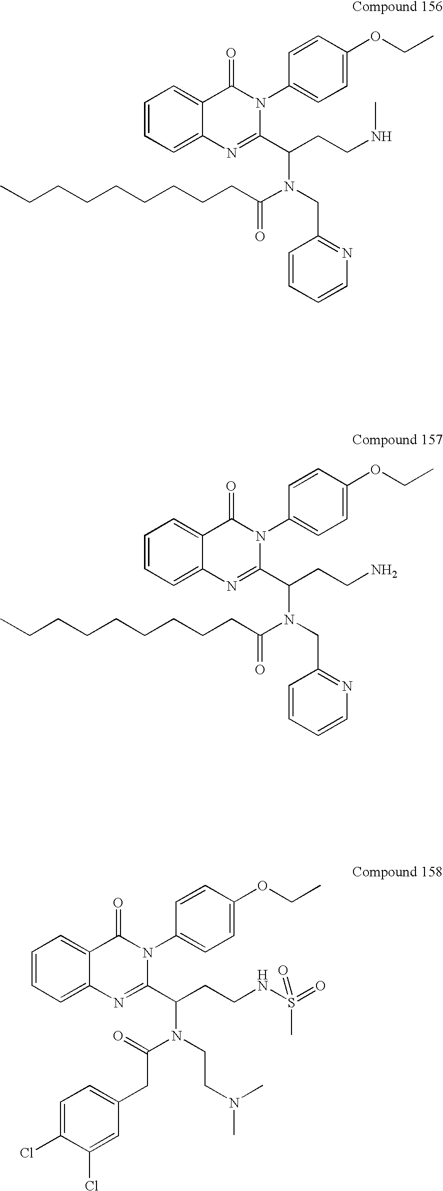

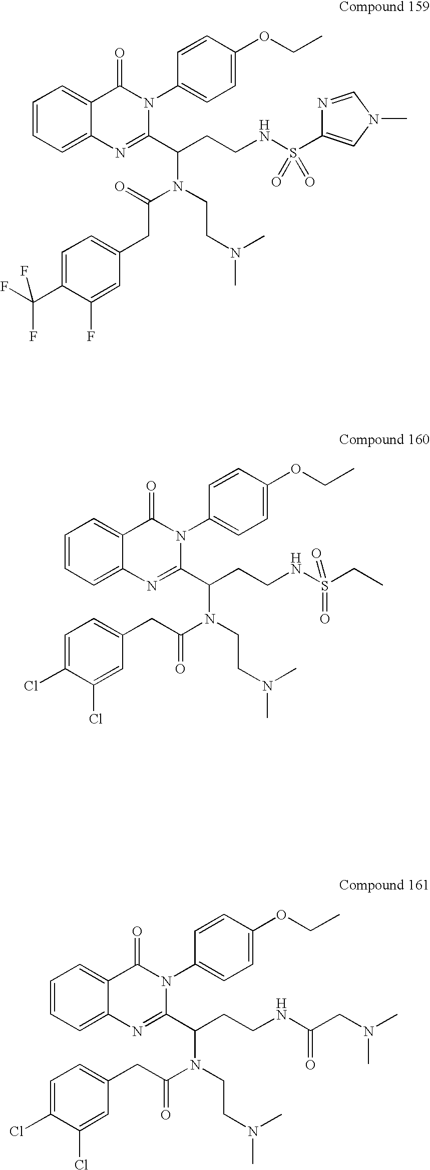

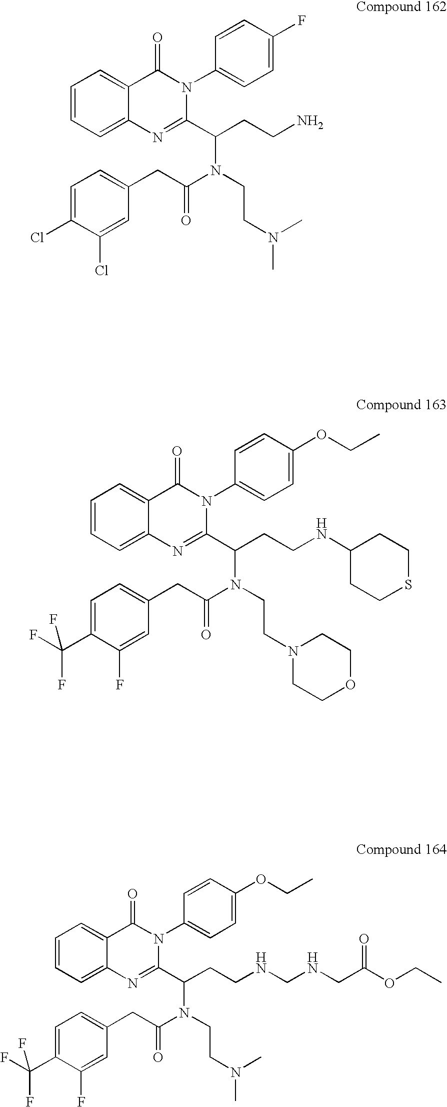

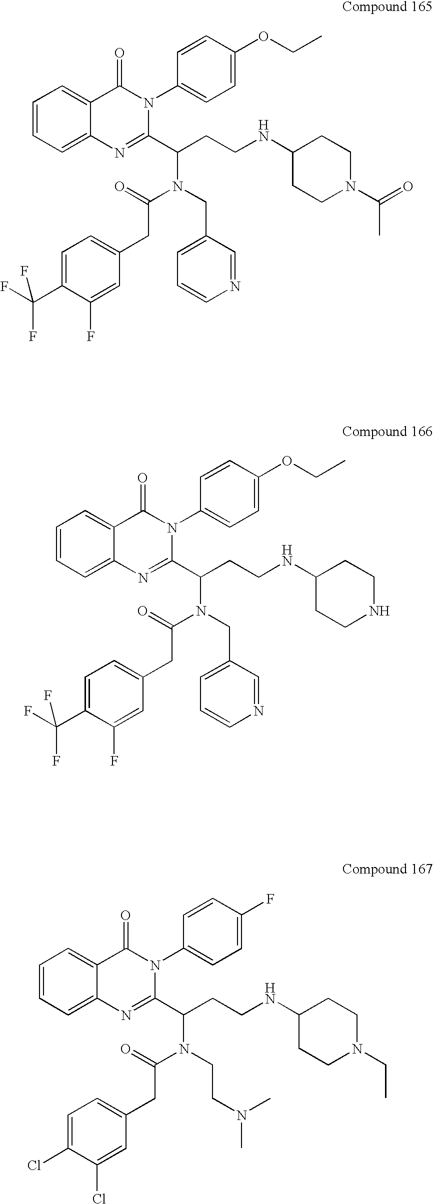

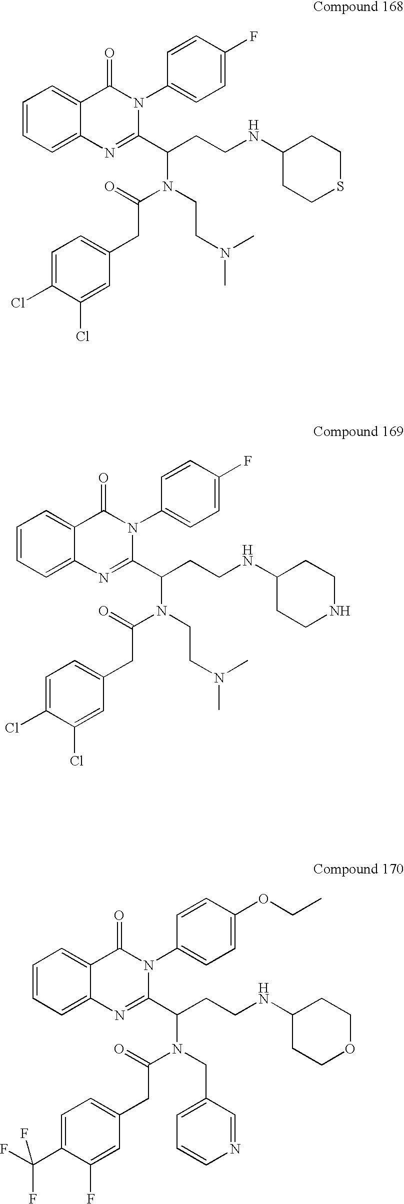

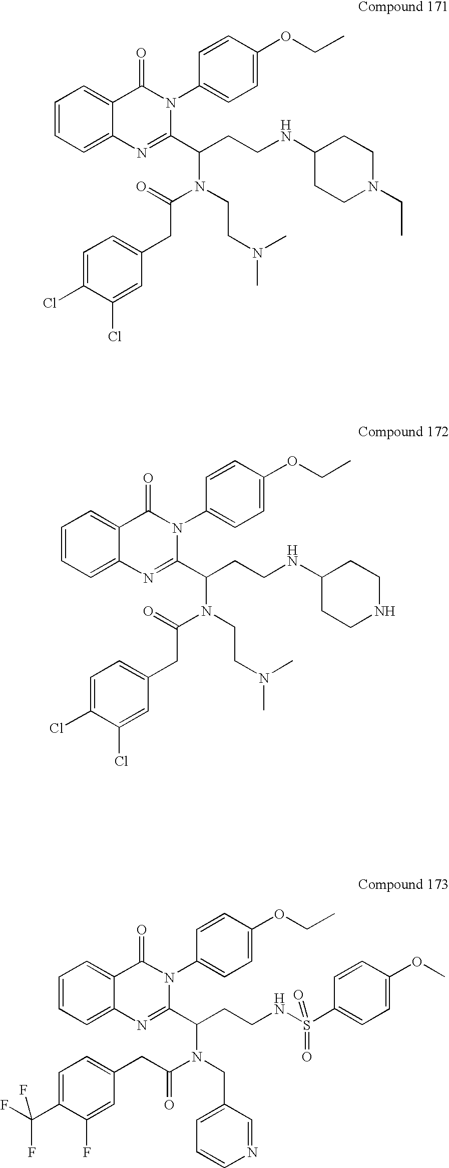

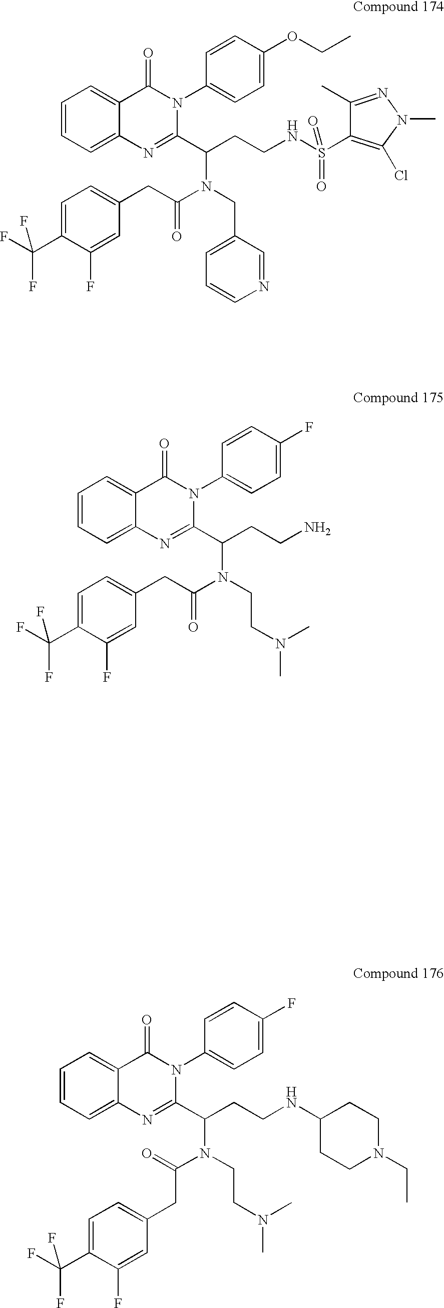

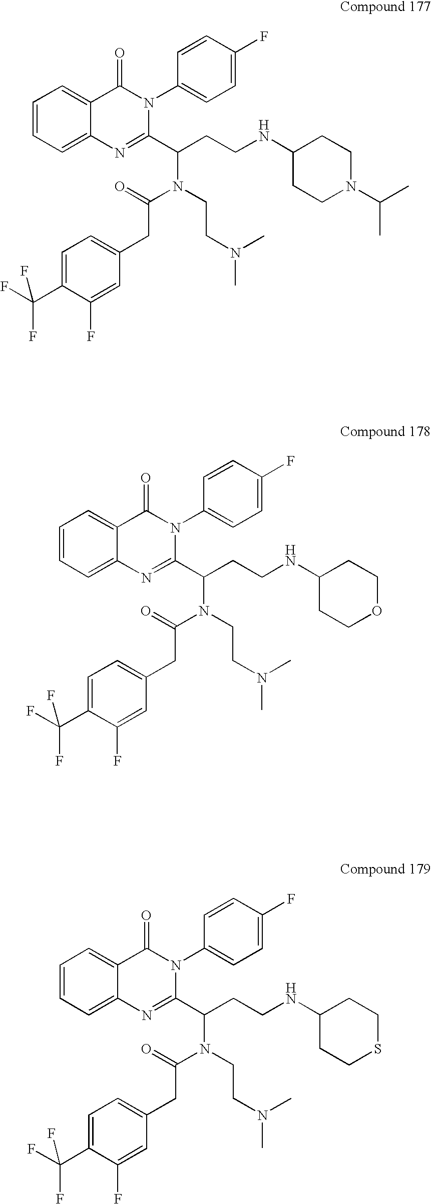

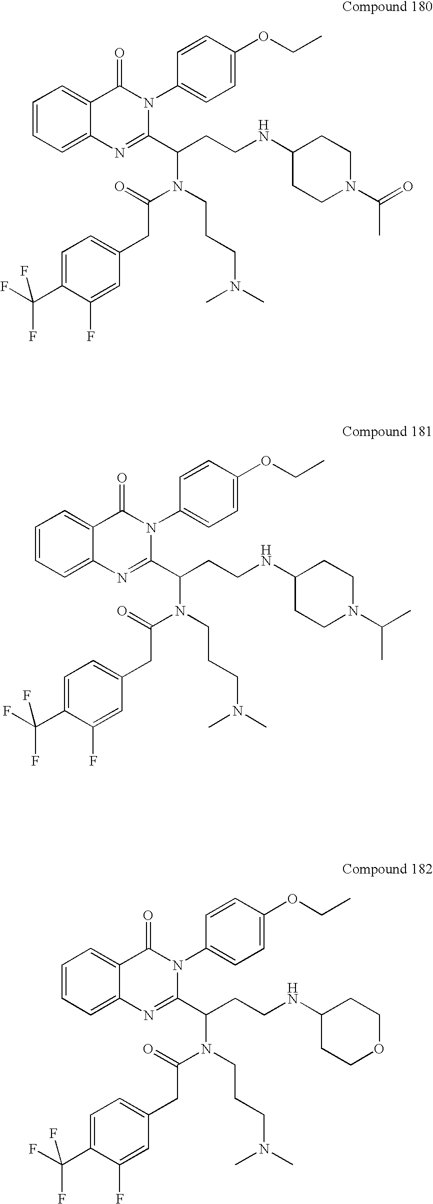

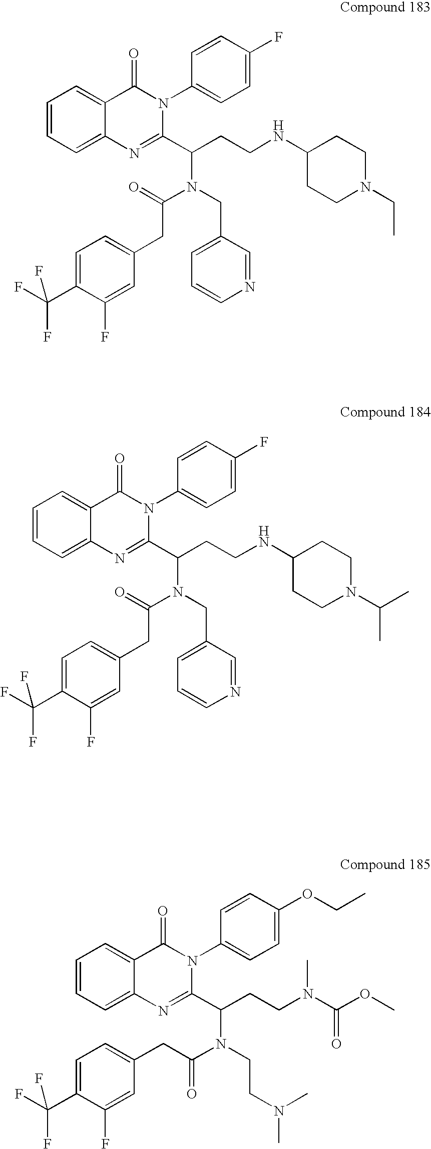

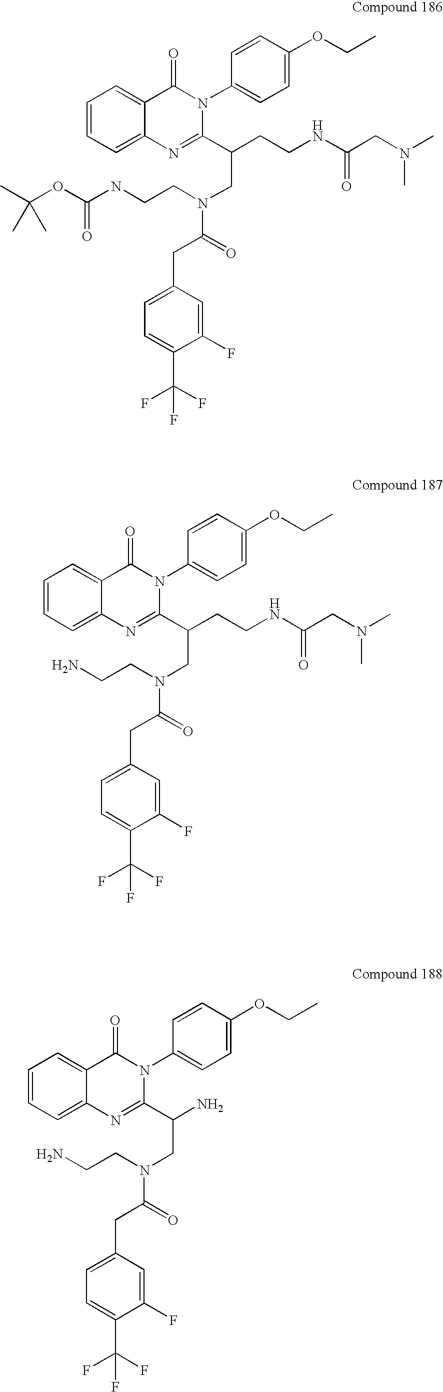

The pyrimidinone compounds described above can be prepared by methods well known in the art, such as those described in U.S. Application 2003/0069234. For example, one can treat anthranilic acid sequentially with an acyl chloride and an amine to obtain a compound having a pyrimidinone ring. The compound thus obtained can then be halogenated and further coupled with a desired amine group. The attached amine group can be further modified to obtain a compound of this invention. In addition, a Lawesson's regent may be used to convert the ketone group on the pyrimidinone ring to a thioketone group. A compound having a pyrimidinone can also be obtained by treating anthranilic acid with a suitable acid. Alternatively, a compound having a pyrimidinone ring can be obtained using 1H-benzo[d][1,3]oxazine-2,4-dione and 1H-quinazoline-2,4-dione as starting materials. Schemes 1-23 described in the Examples below depict the syntheses of some pyrimidinone compounds of this invention. Details of preparation of exemplary compounds 1-188 are provided in Examples 1-188, respectively.

Other pyrimidinone compounds can be prepared using other suitable starting materials following the synthetic routes disclosed herein and other synthetic methods known in the art. These synthetic routes may also additionally include steps, either before or after the steps described specifically herein, to add or remove suitable protecting groups in order to ultimately allow synthesis of the pyrimidinone compounds. In addition, various synthetic steps may be performed in an alternate sequence or order to give the desired compounds. Synthetic chemistry transformations and protecting group methodologies (protection and deprotection) useful in synthesizing applicable pyrimidinone compounds are known in the art and include, for example, those described in R. Larock, Comprehensive Organic Transformations, VCH Publishers (1989); T. W. Greene and P. G. M. Wuts, Protective Groups in Organic Synthesis, 2nd Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

A pyrimidinone compound thus synthesized can be further purified by a known method such as column chromatography, high-pressure liquid chromatography, or recrystallization.

The pyrimidinone compounds mentioned herein may contain a non-aromatic double bond and one or more asymmetric centers. Thus, they can occur as racemates and racemic mixtures, single enantiomers, individual diastereomers, diastereomeric mixtures, and cis- or trans-isomeric forms. All such isomeric forms are contemplated.

Also within the scope of this invention is a pharmaceutical composition contains an effective amount of at least one pyrimidinone compound described above and a pharmaceutical acceptable carrier. Further, this invention covers a method of administering an effective amount of one or more of the pyrimidinone compounds to a patient with an inflammatory or immune disease. Effective doses will vary, as recognized by those skilled in the art, depending on the types of diseases treated, route of administration, excipient usage, and the possibility of co-usage with other therapeutic treatment.

To practice the present invention, a composition having one or more pyrimidinone compounds can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art. A composition having one or more active pyrimidinone compounds can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active pyrimidinone compound. Examples of other carriers include colloidal silicon oxide, magnesium stearate, cellulose, sodium lauryl sulfate, and D&C Yellow # 10.

The pyrimidinone compounds of this invention can be preliminarily screened for their efficacy in treating inflammatory or immune diseases by an in vitro assay (See Example 189 below) and then confirmed by animal experiments and clinical trials. Other methods will also be apparent to those of ordinary skill in the art.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

Compound 1 was prepared following the procedures described below:

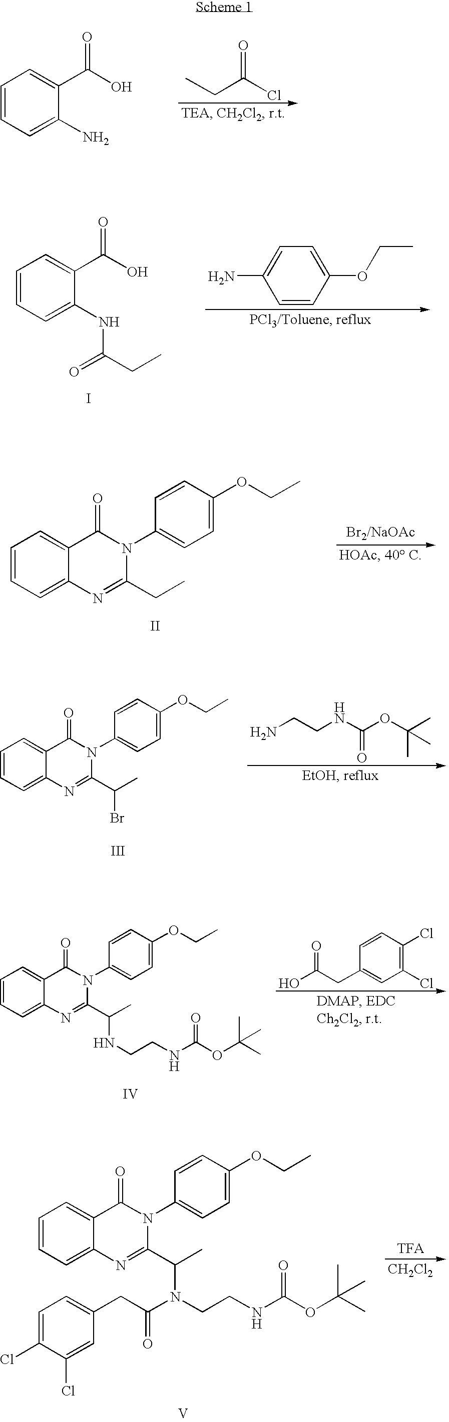

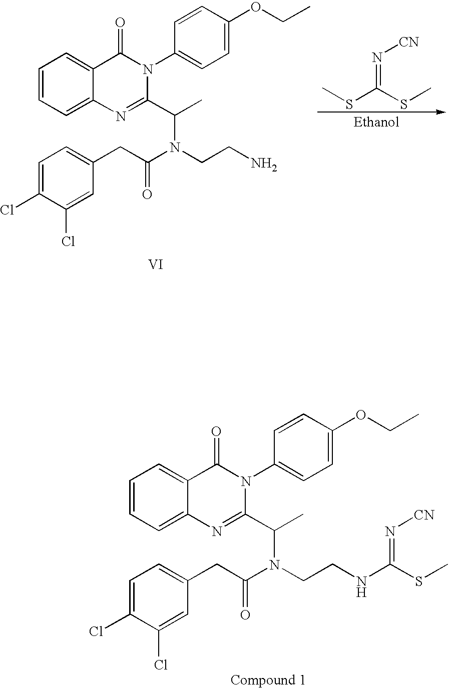

Propionyl chloride (50.5 g, 0.546 mol) was added slowly to a solution of anthranilic acid (50 g, 0.36 mol) and Et₃N (150 mL) in dry dichloromethane (500 mL) through an addition funnel over 1.5 hours at 0° C. Upon completing addition of the propionyl chloride, the reaction mixture was stirred for 17 hours at room temperature and then dichloromethane was removed. The resultant white precipitate was collected via filtration and rinsed with cold water (2×30 mL). The product was then dried in vacuum to afford 1.36 g of Intermediate I.

A solution of phosphorous trichloride (11.2 mL) dissolved in 50 mL toluene was added dropwise to a mixture of Intermediate 1 (24.9 g, 128 mmol) and 4-ethoxylaniline (17.6 g, 128 mmol) suspended in toluene (200 mL) through an addition funnel over 30 minutes. The reaction mixture was kept under reflux for 20 hours and then cooled down to room temperature. The mixture was then quenched with a 10% sodium carbonate aqueous solution (50 mL). The organic layer was separated, dried with magnesium sulfate, and concentrated by vacuum. The crude product was purified by re-crystallization from ethanol to afford 32.2 g of Intermediate II.

A solution of bromine (7.2 g, 44.8 mmol) in glacial acetic acid was added dropwise to a solution of Intermediate II (118 g, 37.4 mmol) and sodium acetate (3.68 g, 44.8 mmol) in glycial acetic acid (220 mL) through an addition funnel over 30 minutes at 40° C. After the addition of the bromine solution, the reaction was stirred for an additional hour. The resultant precipitate was then collected by filtration and dried under vacuum to afford 11.5 g of Intermediate III.

Intermediate III (1.0 g, 2.7 mmol) and (2-amino-ethyl)-carbamic acid tert-butyl ester (0.7 g, 4.0 mmol) were dissolved in 20 mL ethanol and the solution was kept under reflux for 20 hours. The reaction mixture was then concentrated. The crude product thus obtained was purified by column chromatography (silica gel, 5% triethylamine in 1:1 ethyl acetate and n-hexane) to afford 1.0 g of Intermeidate IV.

A catalytical amount of DMAP was added to a solution of (3,4-dichloro-phenyl)-acetic acid (0.37 g, 1.8 mmol) and EDC (0.3 g, 2.24 mmol) in dichloromethane (20 mL). After stirring the above solution for 30 minutes, Intermediate IV (0.68 g, 1.49 mmol) was added. The reaction mixture was stirred at room temperature overnight. It was then diluted with dichloromethane (40 mL) and washed with saturated sodium bicarbonate solution (2×30 mL). The organic layer was separated, dried over magnesium sulfate, filtered, and concentrated under vacuum. The crude product thus obtained was purified by column chromatography (silica gel) to afford 0.8 g of Intermediate V.

To a solution of Intermediate V (0.5 g, 0.78 mmol) in dichloromethane (20 mL) was added trifluoroacetic acid (2.5 mL). The reaction mixture was stirred at room temperature for 3 hours and then concentrated under vacuum. The mixture thus obtained was neutralized with ammonium hydroxide and extracted with dichloromethane. The organic layer was then separated, dried over magnesium sulfate, filtered, and concentrated to afford 0.4 g of Intermediate VI.

Et₃N (0.1 mL) was added to a solution of Intermediate VI (84 mg, 0.16 mmol) and N-cyanoimino-S,S-dimethyl-dithiocarbonate in ethanol (22 mL). The mixture was stirred at room temperature for 4 hours. The crude product was then collected and washed with cool ethanol (2×10 mL) to afford 60 mg of Compound 1.

LC/MS (M+1)⁺: 637.0.

### EXAMPLE 2

Compound 2 was prepared in a manner similar to that described in Example 1. LC/MS (M+1)⁺: 623.1.

### EXAMPLE 3

Compound 3 was prepared following the procedures described below:

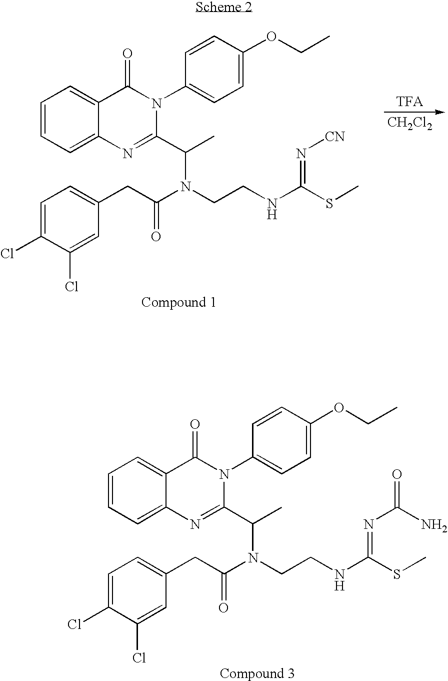

To a solution of Compound 1 (50 mg, 0.078 mmol) in dichloromethane was added trifluoroacetic acid (1.0 mL). The reaction mixture was stirred at room temperature for 3 hours and then concentrated under vacuum. The crude mixture was washed with ether to afford Compound 3 in a salt form.

LC/MS (M+1)⁺: 655.2.

### EXAMPLE 4

Compound 4 was prepared following the procedures described below:

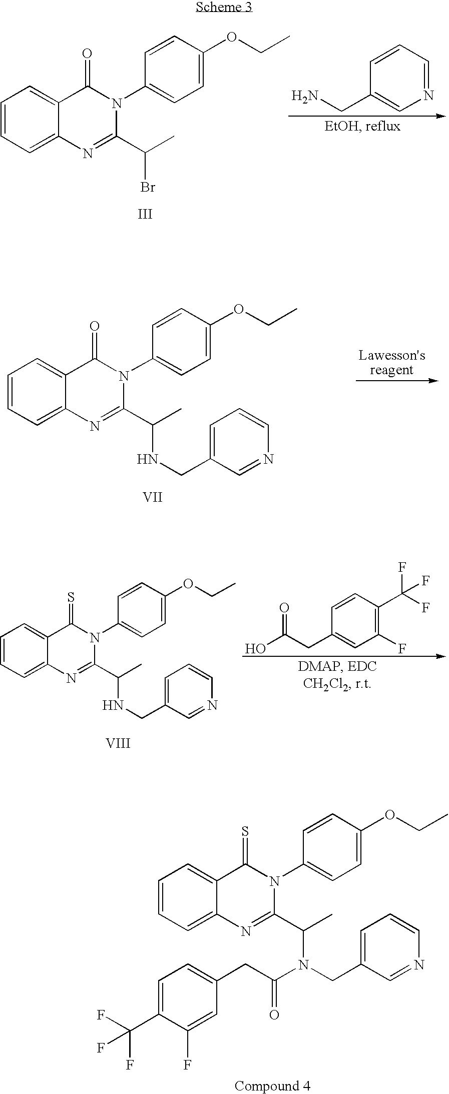

Intermediate VII was prepared from Intermediate III in the manner similar to that of Intermediate IV described in Example 1.

To a solution of Intermediate VII (0.12 g, 0.3 mmol) in dichloromethane was added the Lawesson's reagent (0.13 g, 0.33 mmol). The reaction mixture was refluxed at 120° C. overnight and then concentrated under vacuum. The residue thus obtained was neutralized with ammonium hydroxide and extracted with dichloromethane. The organic layer was separated, dried over magnesium sulfate, filtered, and concentrated to afford 0.1 g of Intermediate VIII.

Compound 4 was prepared from Intermediate VIII in a manner similar to that of Intermediate V described in Example 1.

LC/MS (M+1)⁺: 621.1.

### EXAMPLE 5

Compound 5 was prepared in a manner similar to that described in Example 4.

LC/MS (M+1)⁺: 610.8.

### EXAMPLE 6

Compound 6 was prepared in a manner similar to that described in Example 4.

LC/MS (M+1)⁺: 629.1.

### EXAMPLE 7

Compound 7 was prepared in a manner similar to that described in Example 4.

LC/MS (M+1)⁺: 605.1.

### EXAMPLE 8

Compound 8 was prepared in a manner similar to that described in Example 4.

LC/MS (M+1)⁺: 605.0.

### EXAMPLE 9

Compound 9 was prepared in a manner similar to that described in Example 4.

LC/MS (M+1)⁺: 622.7.

### EXAMPLE 10

Compound 10 was prepared in a manner similar to that described in Example 4.

LC/MS (M+1)⁺: 569.

### EXAMPLE 11

Compound 11 was prepared in a manner similar to that described in Example 4.

LC/MS (M+1)⁺: 587.1.

### EXAMPLE 12

Compound 12 was prepared in a manner similar to that described in Example 4.

LC/MS (M+1)⁺: 618.9.

### EXAMPLE 13

Compound 13 was prepared in a manner similar to that described in Example 4.

LC/MS (M+1)⁺: 622.7.

### EXAMPLE 14

Compound 14 was prepared in a manner similar to that described in Example 4.

LC/MS (M+1)⁺: 637.8.

### EXAMPLE 15

Compound 15 was prepared in a manner similar to that described in Example 4.

LC/MS (M+1)⁺: 612.8.

### EXAMPLE 16

Compound 16 was prepared in a manner similar to that described in Example 4.

LC/MS (M+1)⁺: 655.2.

### EXAMPLE 17

Compound 17 was prepared in a manner similar to that described in Example 4.

LC/MS (M+1)⁺: 644.9.

### EXAMPLE 18

Compound 18 was prepared in a manner similar to that described in Example 4.

LC/MS (M+1)⁺: 613.2.

### EXAMPLE 19

Compound 19 was prepared in a manner similar to that described in Example 4.

LC/MS (M+1)⁺: 643.2.

### EXAMPLE 20

Compound 20 was prepared following the procedures described below:

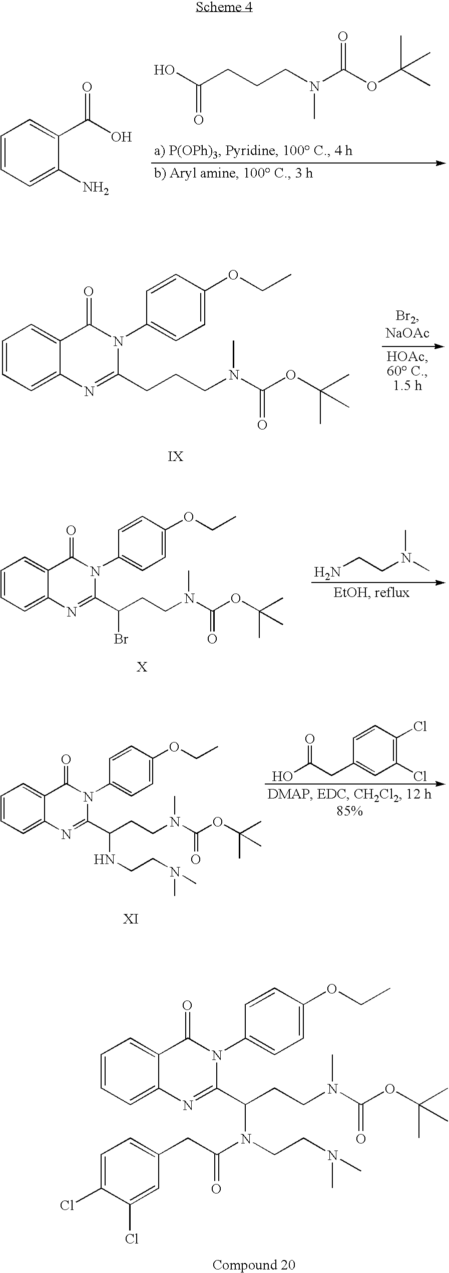

3 mL triphenylphosphite (22 g, 70 mmol) was added to a solution of anthranilic acid (8.0 g, 58.6 mmol) and 4-(tert-butoxycarbonyl-methyl-amino)-butyric acid (12.7 g, 58.6 mmol) in 100 mL of anhydrous pryridine at room temperature. The resultant yellow solution was stirred at 100° C. for 4 hours. 4-Ethoxylaniline (8.8 g, 64 mmol) was then added and the reaction mixture was stirred for another 3 hours at 100° C. The mixture was then cooled down to room temperature and concentrated under vacuum to give a brown residue. The residue was sequentially washed with 1N HCl (2×10 mL) and saturated sodium bicarbonate (2×10 mL), and then extracted with CH₂Cl₂ (3×30 mL). The organic layer was separated, dried over magnesium sulfate, and concentrated under vacuum to give a brown residue. The residue thus obtained was purified by silica gel chromatography to afford 12.5 of Intermediate IX.

To a solution of Intermediate IX (4.8 g, 11.0 mmol) and sodium acetate (1.0 g, 12.1 mmol) dissolved in 70 mL glycial acetic acid at 60° C. was added dropwise a solution of bromine (1.7 g, 11.0 mmol) in glacial acetic acid through an addition funnel over 15 minutes. After the addition of the bromine solution, the reaction was stirred for an additional 30 minutes. The reaction solution was then poured into water (200 mL). The resultant mixture was stirred at room temperature for 30 minutes and then extracted with CH₂Cl₂, dried over magnesium sulfate, filtered, and concentrated under vacuum to give a solid. The solid thus obtained was purified by silica gel chromatography to afford 3.4 g of Intermediate X.

Intermediate IX was prepared from Intermediate X in a manner similar to that of Intermediate IV described in Example 1.

Compound 20 was prepared from Intermediate IX in a manner similar to that Intermediate V described in Example 1.

LC/MS (M+1)⁺: 710.3.

### EXAMPLE 21

Compound 21 was prepared following the procedures described below:

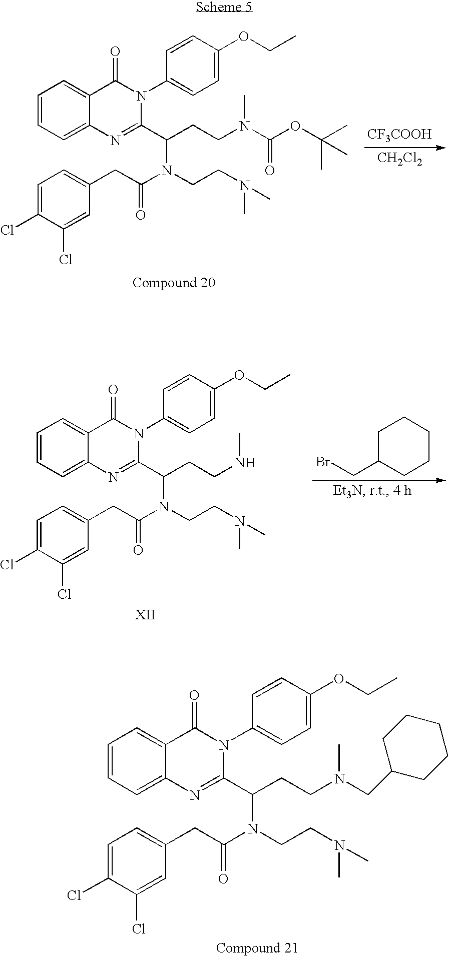

Intermediate XII was prepared from compound 20 in the manner similar to that of Intermediate VI described in Example 1.

To a solution of Intermediate XII (61 mg, 1.0 mmol) in dichloromentane (10 mL) was added bromomethyl-cyclohexane (17.7 mg, 1.0 mmol) and an excess amount of triethyl amine at room temperature. The reaction mixture was stirred for additional 4 hours. The reaction was then quenched with 1.0 N NaOH and extracted with dichloromethane (30 mL×2). The organic layer was separated, dried over magnesium sulfate, and concentrated under vacuum to give a brown residue. The brown residue was then purified by silica gel chromatography to give 54 mg of Compound 21.

LC/MS (M+1)⁺: 706.3.

### EXAMPLE 22

Compound 22 was prepared in a manner similar to that described in Example 21.

LC/MS (M+1)⁺: 624.1.

### EXAMPLE 23

Compound 23 was prepared in a manner similar to that described in Example 20.

LC/MS (M+1)⁺: 728.3.

### EXAMPLE 24

Compound 24 was prepared in a manner similar to that described in Example 21.

LC/MS (M+1)⁺: 706.3.

### EXAMPLE 25

Compound 25 was prepared in a manner similar to that of Intermediate XII described in Example 21.

LC/MS (M+1)⁺: 628.2.

### EXAMPLE 26

Compound 26 was prepared in a manner similar to that described in Example 21.

LC/MS (M+1)⁺: 700.2.

### EXAMPLE 27

Compound 27 was prepared in a manner similar to that described in Example 21.

LC/MS (M+1)⁺: 718.2.

### EXAMPLE 28

Compound 28 was prepared in a manner similar to that described in Example 20.

LC/MS (M+1)⁺: 696.2.

### EXAMPLE 29

Compound 29 was prepared in a manner similar to that of Intermediate XII described in Example 21.

LC/MS (M+1)⁺: 596.2.

### EXAMPLE 30

Compound 30 was prepared in a manner similar to that described in Example 21.

LC/MS (M+1)⁺: 736.4.

### EXAMPLE 31

Compound 31 was prepared in a manner similar to that described in Example 20.

LC/MS (M+1)⁺: 710.2.

### EXAMPLE 32

Compound 32 was prepared in a manner similar to that described in Example 20.

LC/MS (M+1)⁺: 728.3.

### EXAMPLE 33

Compound 33 was prepared in a manner similar to that described in Example 20.

LC/MS (M+1)⁺: 682.2.

### EXAMPLE 34

Compound 34 was prepared in a manner similar to that described in Example 21.

LC/MS (M+1)⁺: 780.2.

### EXAMPLE 35

Compound 35 was prepared in a manner similar to that described in Example 21.

LC/MS (M+1)⁺: 726.3.

### EXAMPLE 36

Compound 36 was prepared in a manner similar to that described in Example 21.

LC/MS (M+1)⁺: 710.3.

### EXAMPLE 37

Compound 37 was prepared in a manner similar to that described in Example 20.

LC/MS (M+1)⁺: 848.3.

### EXAMPLE 38

Compound 38 was prepared in a manner similar to that described in Example 21.

LC/MS (M+1)⁺: 699.3.

### EXAMPLE 39

Compound 39 was prepared in a manner similar to that described in Example 20.

LC/MS (M+1)⁺: 700.3.

### EXAMPLE 40

Compound 40 was prepared in a manner similar to that described in Example 20.

LC/MS (M+1)⁺: 744.3.

### EXAMPLE 41

Compound 41 was prepared in a manner similar to that described in Example 20.

LC/MS (M+1)⁺: 762.3.

### EXAMPLE 42

Compound 42 was prepared in a manner similar to that described in Example 20.

LC/MS (M+1)⁺: 738.3.

### EXAMPLE 43

Compound 43 was prepared in a manner similar to that described in Example 20.

LC/MS (M+1)⁺: 756.3.

### EXAMPLE 44

Compound 44 was prepared in a manner similar to that described in Example 20.

LC/MS (M+1)⁺: 762.4.1.

### EXAMPLE 45

Compound 45 was prepared following the procedures described below:

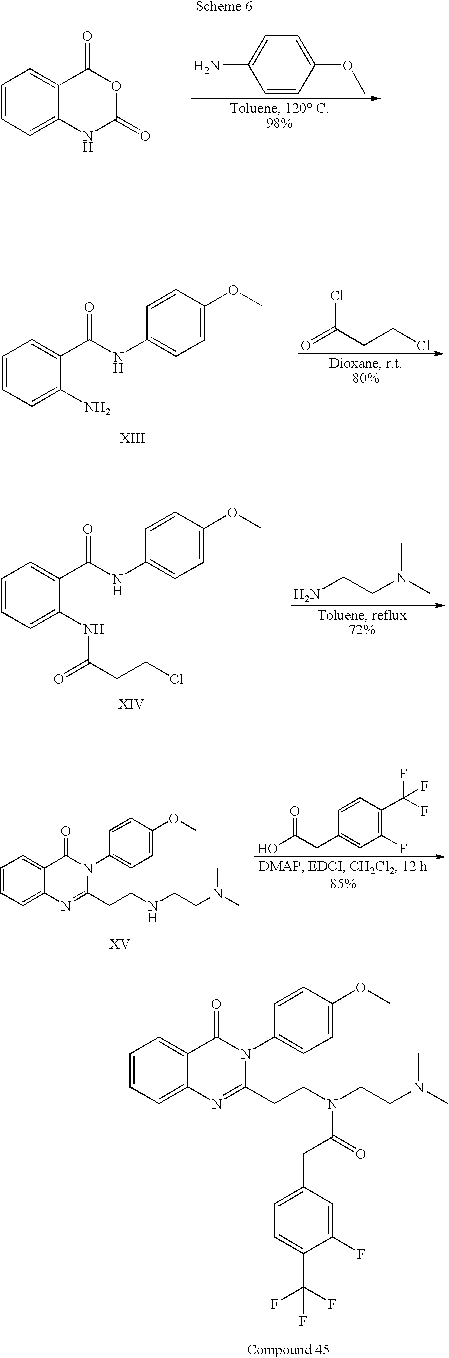

1H-benzo[d][1,3]oxazine-2,4-dione (17.4 g, 110 mmol) and 4-ethoxy-phenylamine (19.0 g, 116.0 mmol) were dissolved in toluene (120 mL). The reaction mixture was kept under reflux for 8 hours. It was then cooled down to room temperature and concentrated under vacuum to give a brown residue. The residue was washed with saturated sodium bicarbonate (2×10 mL) and extracted with CH₂Cl₂ (3×30 mL). The organic layer was separated, dried over magnesium sulfate, and concentrated under vacuum to give a crude product. The crude product was then purified by silica gel chromatography to give 25.9 g of Intermediate XIII.

Intermediate XIII (4.7 g, 19.3 mmol) and 3-chloro-propionyl chloride (2.7 g, 21.2 mmol) were mixed in dioxane (20 mL) at 0° C. The mixture was then stirred for 5 hours at room temperature and was poured into water (200 mL). The resultant precipitate was filtered and dried under vacuum to give 6.0 g of Intermediate XIV.

Intermediate XIV (0.2 g, 0.6 mmol) and 2-dimethylamino-ethylamine (0.1 mL, 0.9 mmol) was dissolved in toluene (20 mL). The mixture was kept under reflux for 8 hours, cooled down to room temperature, and concentrated under vacuum to give a brown residue. The residue was washed with saturated sodium bicarbonate (2×10 mL) and extracted with CH₂Cl₂ (3×30 mL). The organic layer was separated, dried over magnesium sulfate, and concentrated to give a crude product. The crude product was purified by silica gel chromatography to give 0.17 g of Intermediate XV.

Compound 45 was prepared from Intermediate XV in the manner similar to that of Intermediate V described in Example 1.

LC/MS (M+1)⁺: 585.2.

### EXAMPLE 46

Compound 46 was prepared in a manner similar to that described in Example 45.

LC/MS (M+1)⁺: 684.2.

### EXAMPLE 47

Compound 47 was prepared in a manner similar to that described in Example 45.

LC/MS (M+1)⁺: 590.8.

### EXAMPLE 48

Compound 48 was prepared in a manner similar to that described in Example 45.

LC/MS (M+1)⁺: 590.8.

### EXAMPLE 49

Compound 49 was prepared in a manner similar to that described in Example 45.

LC/MS (M+1)⁺: 680.1.

### EXAMPLE 50

Compound 50 was prepared in a manner similar to that described in Example 45.

LC/MS (M+1)⁺: 591.1.

### EXAMPLE 51

Compound 51 was prepared in a manner similar to that described in Example 45.

LC/MS (M+1)⁺: 572.8.

### EXAMPLE 52

Compound 52 was prepared in a manner similar to that described in Example 45.

LC/MS (M+1)⁺: 572.8.

### EXAMPLE 53

Compound 53 was prepared in a manner similar to that described in Example 45.

LC/MS (M+1)⁺: 572.8.

### EXAMPLE 54

Compound 54 was prepared in a manner similar to that described in Example 45.

LC/MS (M+1)⁺: 561.8.

### EXAMPLE 55

Compound 55 was prepared following the procedures described below:

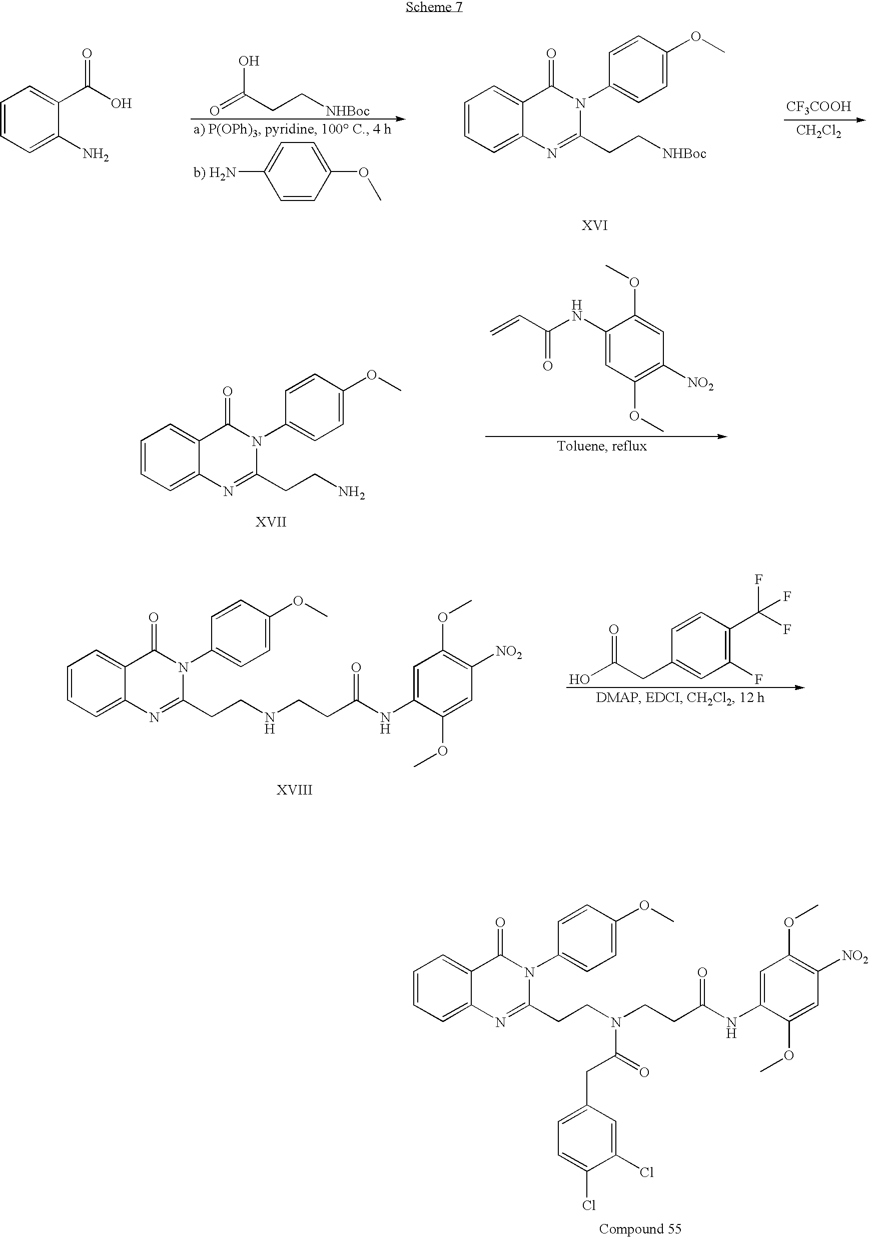

Intermediate XVI was prepared in the manner similar to Intermediate IX described in Example 20. Intermediate XVII was prepared from Intermediate XVI obtained above in the manner similar to Compound 3.

Intermediate XVII (0.29 g, 10.0 mmol) obtained above and N-(2,5-dimethoxy-4-nitro-phenyl)-acrylamide (0.28 g, 11.0 mmol) were dissolved in ethanol (20 mL). The mixture was kept under reflux for 12 hours, cooled down to room temperature, and concentrated under vacuum to give a brown residue. The residue was purified by silica gel chromatography to give 0.46 g of Intermediate XVIII.

Compound 55 was prepared from Intermediate XVIII obtained above in the manner similar to Intermediate V described in Example 1.

LC/MS (M+1)⁺: 807.7.

### EXAMPLE 56

Compound 56 was prepared in a manner similar to that described in Example 55.

LC/MS (M+1)⁺: 792.1.

### EXAMPLE 57

Compound 57 was prepared in a manner similar to that described in Example 45.

LC/MS (M+1)⁺: 641.6.

### EXAMPLE 58

Compound 58 was prepared in a manner similar to that described in Example 45.

LC/MS (M+1)⁺: 550.9.

### EXAMPLE 59

Compound 59 was prepared in a manner similar to that described in Example 45.

LC/MS (M+1)⁺: 626.9.

### EXAMPLE 60

Compound 60 was prepared in a manner similar to that described in Example 45.

LC/MS (M+1)⁺: 592.8.

### EXAMPLE 61

Compound 61 was prepared in a manner similar to that described in Example 45.

LC/MS (M+1)⁺: 610.9.

### EXAMPLE 62

Compound 62 was prepared in a manner similar to that described in Example 45.

LC/MS (M+1)⁺: 576.9.

### EXAMPLE 63

Compound 63 was prepared in a manner similar to that described in Example 45.

LC/MS (M+1)⁺: 584.9.

### EXAMPLE 64

Compound 64 was prepared in a manner similar to that described in Example 45.

LC/MS (M+1)⁺: 551.1.

### EXAMPLE 65

Compound 65 was prepared in a manner similar to that described in Example 45.

LC/MS (M+1)⁺: 576.9.

### EXAMPLE 66

Compound 66 was prepared in a manner similar to that described in Example 45.

LC/MS (M+1)⁺: 605.9.

### EXAMPLE 67

Compound 67 was prepared in a manner similar to that described in Example 45.

LC/MS (M+1)⁺: 604.9.

### EXAMPLE 68

Compound 68 was prepared in a manner similar to that described in Example 45.

LC/MS (M+1)⁺: 639.2.

### EXAMPLE 69

Compound 69 was prepared in a manner similar to that described in Example 45.

LC/MS (M+1)⁺: 605.2.

### EXAMPLE 70

Compound 70 was prepared in a manner similar to that described in Example 45.

LC/MS (M+1)⁺: 611.2.

### EXAMPLE 71

Compound 71 was prepared in a manner similar to that described in Example 45.

LC/MS (M+1)⁺: 557.2.

### EXAMPLE 72

Compound 72 was prepared in a manner similar to that described in Example 45.

LC/MS (M+1)⁺: 657.3.

### EXAMPLE 73

Compound 73 was prepared in a manner similar to that described in Example 45.

LC/MS (M+1)⁺: 543.2.

### EXAMPLE 74

Compound 74 was prepared in a manner similar to that described in Example 45.

LC/MS (M+1)⁺: 637.1.

### EXAMPLE 75

Compound 75 was prepared in a manner similar to that described in Example 45.

LC/MS (M+1)⁺: 651.2.

### EXAMPLE 76

Compound 76 was prepared in a manner similar to that described in Example 45.

LC/MS (M+1)⁺: 651.7.

### EXAMPLE 77

Compound 77 was prepared in a manner similar to that described in Example 45.

LC/MS (M+1)⁺: 647.2.

### EXAMPLE 78

Compound 78 was prepared following the procedures described below:

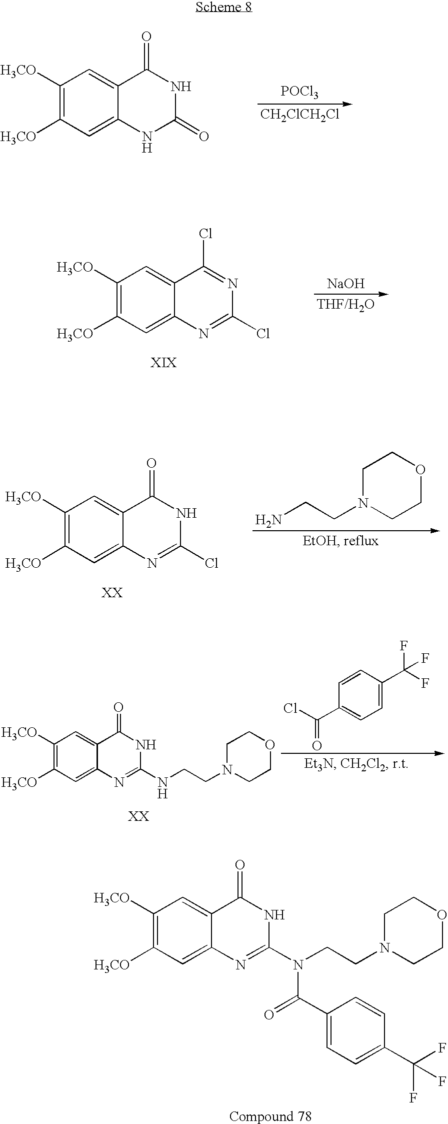

6,7-dimethoxy-1H-quinazoline-2,4-dione (22.2 g, 100 mmol) and POCl₃ (20 mL) were added in 1,2-dichloroethane (30 mL) and the mixture was kept under reflux for 3 hours. Subsequently, the mixture was poured into ice water. The precipitate thus obtained was filtered and dried under vacuum to give 17.0 g of Intermediate XIX.

Intermediate XIX (2.6 g, 10.0 mmol) and an excess amount of 1.0 N NaOH aqueous solution (20 mL) were added in tetrahydrofuran (100 mL). The mixture was stirred at room temperature for 2 hours. The organic solvent was removed by vacuum.

The solid thus obtained was filtered, washed with water, and dried under vacuum to give 2.1 g of Intermediate XX.

Intermediate XX (2.4 g, 10.0 mmol) and 2-morpholin-4-yl-ethylamine (1.9 g, 15.0 mmol) were dissolved in EtOH (50 mL). The reaction mixture was kept under reflux for 8 hours, cooled down to room temperature, and concentrated under vacuum to give a brown residue. The residue was purified by silica gel chromatography to give 3.1 g of Intermediate XXI.

An excess amount of triethylamine was added to a solution of Intermediate XXI (66.8 mg, 0.2 mmol) and 4-trifluoromethyl-benzoyl chloride (62.4 mg, 0.3 mmol) in dichloromethane (30 mL). The reaction mixture was stirred at room temperature for 4 hours. The mixture was then washed with saturated sodium bicarbonate (2×10 mL) and extracted with CH₂Cl₂ (3×30 mL). The organic layer was separated, dried over magnesium sulfate, and concentrated under vacuum to give a brown residue. The residue was purified by silica gel chromatography to give 62 mg of Compound 78.

LC/MS (M+1)⁺: 507.3.

### EXAMPLE 79

Compound 79 was prepared following the procedures described below:

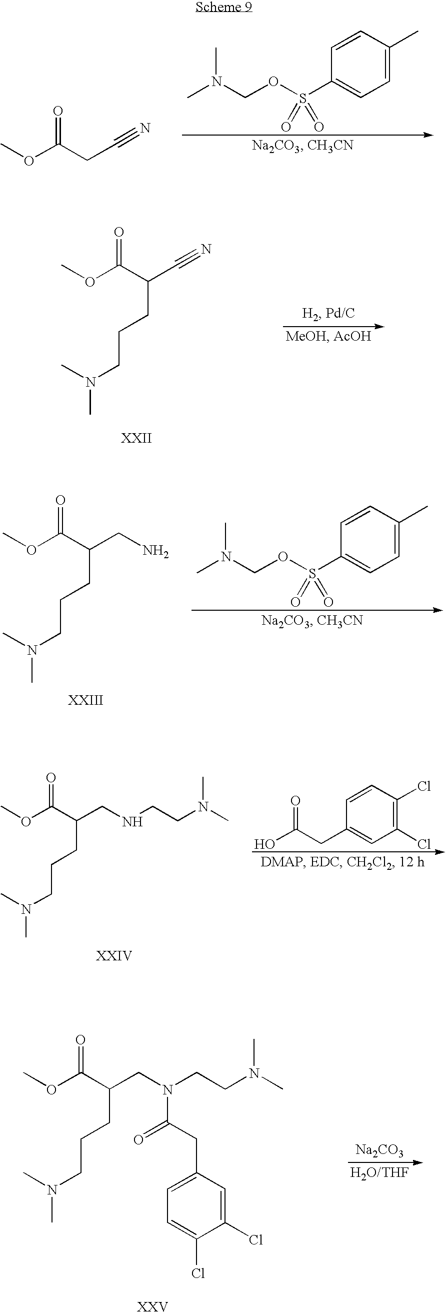

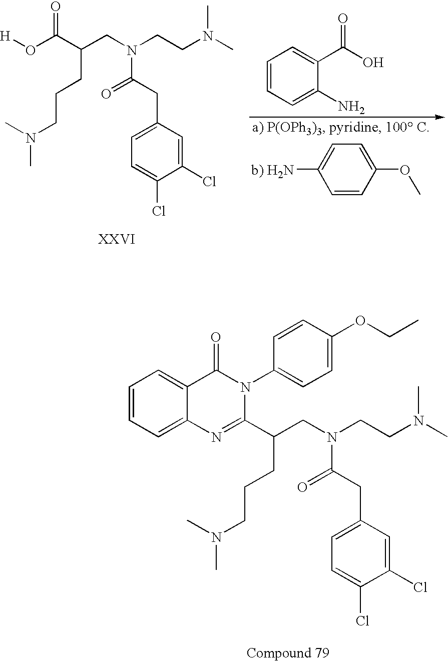

An excess amount of Na₂CO₃ was added to a solution of cyano-acetic acid methyl ester (1.8 g, 20 mmol) and toluene-4-sulfonic acid 3-dimethylamino-propyl ester (5.1 g, 20 mmol) in acetonitrile (100 mL) at room temperature. The reaction mixture was kept under reflux for 3 hours. Subsequently, the mixture was filtered and extracted with ether. The organic layer was combined and concentrated under vacuum to give a brown residue. The brown residue was then purified by silica gel chromatography to give 3.9 g of Intermediate XXII.

To a solution of Intermediate XXII (1.8 g, 10.0 mmol) in methanol (50 mL) was added a catalytic amount of 10% Pd/C and acetic acid after the flask was purged with N₂. The flask was then filled with H₂ gas up to 70 psi and was kept at this pressure for 12 hours. The reaction mixture was filtered and concentrated under vacuum to give a light yellow residue. The residue was then purified by silica gel chromatography to give 1.4 g of Intermediate XXIII.

Intermediate XXIV was prepared in a manner similar to Intermediate XXII described above.

Intermediate XXV was prepared in a manner similar to Intermediate V described in Example 1.

Intermediate XXV (2.2 g, 5.0 mmol) and an excess amount of Na₂CO₃ were dissolved in a mixture of H₂O and THF (1/1, 50 mL). The reaction mixture was stirred at room temperature for 6 hours and extracted with ether. The organic layer was separated and concentrated under vacuum to give a brown residue. The residue was purified by silica gel chromatography to give 1.9 g of Intermediate XXVI.

Compound 79 was prepared in the manner similar to Intermediate IX described in Example 20.

LC/MS (M+1)⁺: 652.7.

### EXAMPLE 80

Compound 80 was prepared following the procedures described below.

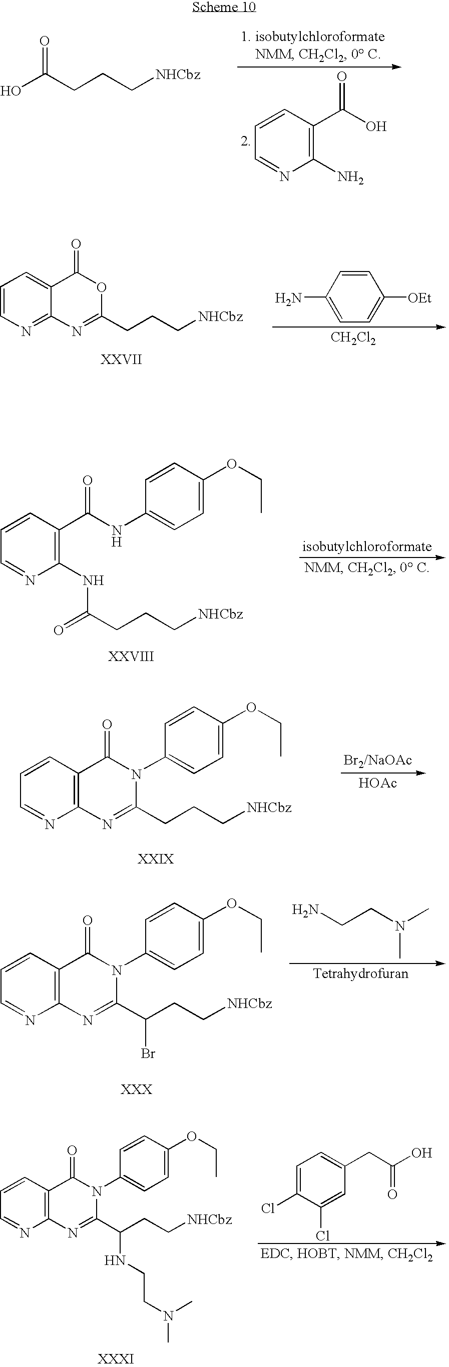

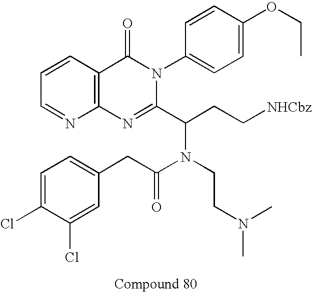

Isobutylchloroformate (25.9 mL, 200 mmol) and N-methyl morpholine (27.5 mL, 250 mmol) were slowly added to a solution of 4-benzyloxycarbonylamino-butyric acid (23.72 g, 100 mmol) in dry dichloromethane (DCM, 250 mL) at 0° C. via an addition funnel over 0.5 hour. After the addition was complete, the mixture was allowed to stir for 45 minutes and 2-aminonicotinic acid (12.81 g, 100 mmol) was added to the mixture. The mixture thus obtained was stirred at room temperature overnight, diluted with 0.5 L DCM, and washed with 1.0 N HCl (200 mL) and brine (100 mL). The organic layer was separated, dried with magnesium sulfate, concentrated under vacuum. Intermediate XXVII was obtained and used in the next step without further purification.

p-Phenetidine (12.9 mL, 100 mmol) was added to a solution of crude Intermediate XXVII in 400 mL DCM at 0° C. over 5 minutes. The solution was stirred at room temperature overnight. It was then diluted with 0.5 L DCM and washed sequentially with 1.0 N HCl (200 mL), saturated NaHCO₃ (200 mL), and brine (200 mL). The organic layer was separated, dried with magnesium sulfate, filtered, and concentrated under vacuum. Intermediate XXVIII was obtained and used in the next step without further purification.

N-Methyl morpholine (13.2 mL, 120 mmol) and iso-butylchloroformate (13.0 mL, 100 mmol) were added to a solution of crude Intermediate XXVIII in 500 mL DCM at 0° C. over 5 minutes. The solution was stirred at room temperature overnight. It was then diluted with 1 L DCM and washed sequentially with 1.0 N HCl (200 mL), saturated NaHCO₃ (200 mL), and brine (200 mL). The organic layer was separated, dried with magnesium sulfate, filtered, and concentrated under vacuum. The residue thus obtained was purified by silica gel chromatography to give Intermediate XXIX (18.3 g).

A mixture of Intermediate XXIX (3.6 g, 7.9 mmol) and sodium acetate (0.78 g, 9.5 mmol) was dissolved in 40 mL glacial acetic acid at 40° C. A solution of bromine (1.26 g, 79 mmol) in glacial acetic acid was then added via an addition funnel over 30 minutes. After the addition of the bromine solution, the solution thus obtained was stirred an additional hour and poured into 400 mL water. The mixture was then stirred for 1 hour. The precipitate was collected by filtration and dried under vacuum to afford Intermediate XXX (4.1 g).

A solution of Intermediate XXX (1.0 g, 1.9 mmol) and N,N-dimethylethylenediamine (0.66 g, 7.4 mmol) dissolved in 20 mL THF was heated to 40° C. for 17 hours. THF was then removed under vacuum. The crude product thus obtained was purified by column chromatography on silica gel to afford Intermediate XXXI (0.6 g).

EDC (0.63 g, 3.3 mmol), HOBt (0.22 g, 1.7 mmol) and N-methyl morphorine (0.36 mL, 3.3 mmol) were added to a solution of (3,4-dichlorophenyl)acetic acid (0.29 g, 1.4 mmol) in 10 mL dichloromethane. After stirring the solution for 30 minutes, Intermediate XXXI (0.6 g, 1.1 mmol) was added and the solution was stirred at room temperature for overnight. The reaction mixture was then diluted with dichloromethane (20 mL) and washed with a saturated sodium bicarbonate solution (2×30 mL). The organic layer was separated, dried over magnesium sulfate, filtrated, and concentrated under vacuum. The crude solid thus obtained was recrystallized with ethanol to afford Compound 80 (0.6 g).

LC/MS (M+1)⁺: 731.2.

### EXAMPLE 81

Compound 81 was prepared following the procedures described below.

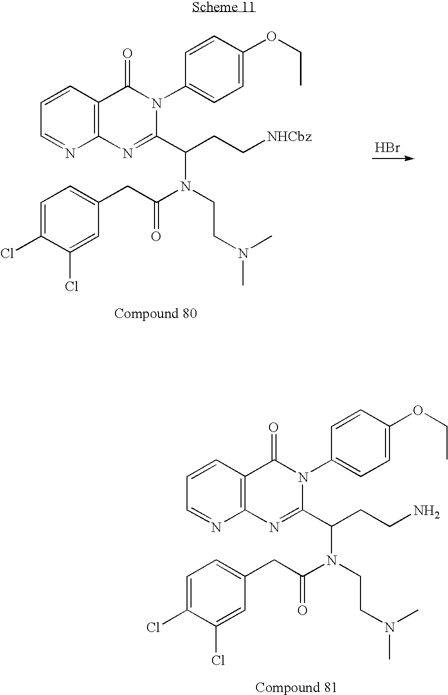

A mixture of Compound 80 (0.6 g, 0.82 mmol) and 33% HBr in HOAc (15 mL) was stirred at room temperature for 4 hours. The reaction mixture was then diluted with ether (25 mL), filtered, washed with a mixture of MeOH and ether, dried to give Compound 81 (0.6 g).

LC/MS (M+1)⁺: 597.

### EXAMPLE 82

Compound 82 was prepared following the procedures described below.

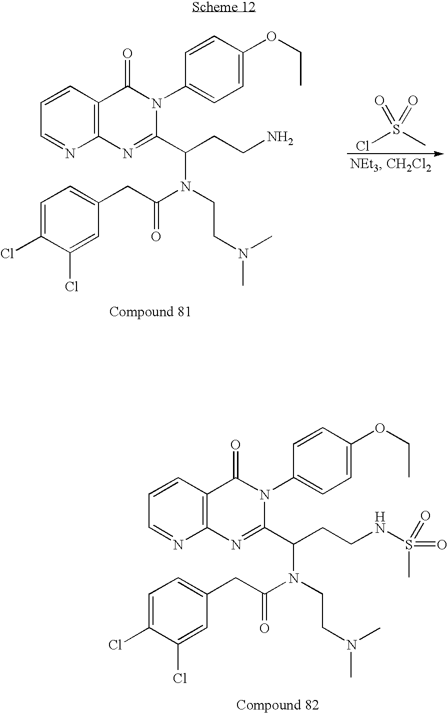

Methanesulfonyl chloride (0.03 g, 0.26 mmol) was added dropwise to a solution of Compound 81 (0.6 g, 0.17 mmol) and Et₃N (0.17 g, 1.7 mmol) in dichloromethane (10 mL) at 0° C. over a period of 10 minutes. The mixture was stirred at room temperature for overnight and then washed sequentially with 1 N HCl (10 mL) and water (20 mL). The organic layer was separated, dried over magnesium sulfate, filtered, and concentrated under vacuum. The crude product thus obtained was purified by silica gel chromatography to give Compound 82 (0.09 g).

LC/MS (M+1)⁺: 675.2.

### EXAMPLE 83

Compound 83 was prepared following the procedures described below.

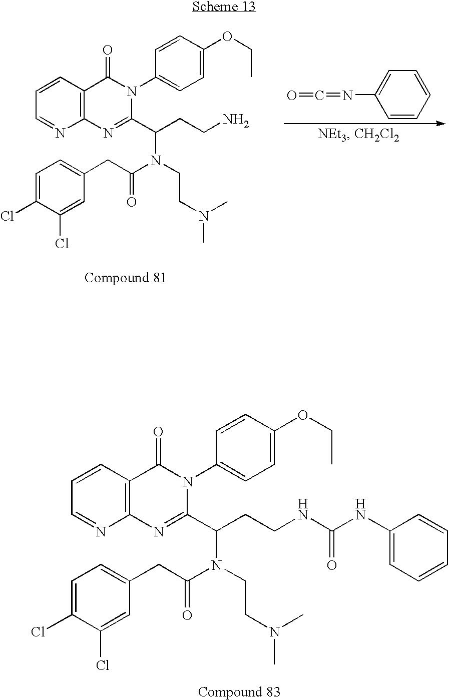

Phenyl isocyanate (0.1 g, 0.84 mmol) was added dropwise to a solution of Compound 81 (0.1 g, 0.17 mmol) and Et₃N (0.03 g, 0.34 mmol) in dichloromethane (5 mL) at 0° C. over a period of 10 minutes. The mixture was stirred at room temperature for 3 hours and then washed sequentially with 1 N HCl (10 mL) and water (20 mL). The organic layer was separated, dried over magnesium sulfate, filtered, and concentrated under vacuum. The crude product thus obtained was purified by silica gel chromatography to give Compound 83 (0.1 g).

LC/MS (M+1)⁺: 716.3.

### EXAMPLE 84

Compound 84 was prepared following the procedures described below.

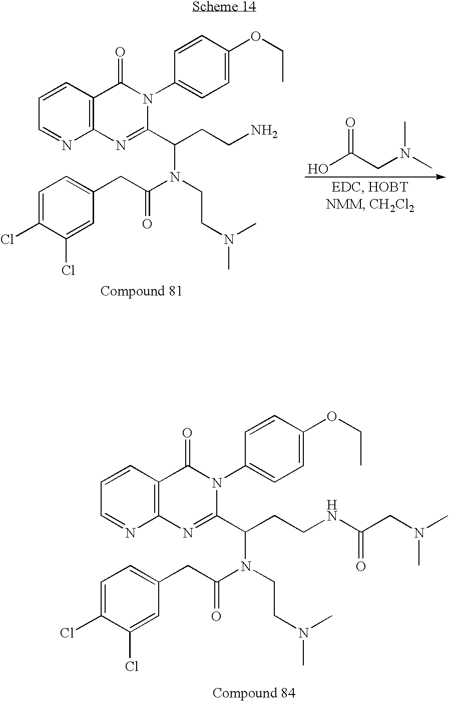

EDC (0.12 g, 0.63 mmol), HOBt (0.05 g, 0.32 mmol) and excess amount of N-methyl morphorine (0.06 g, 0.6 mmol) were added to a solution of dimethylamino acetic acid (0.04 g, 0.25 mmol) in 5 mL dichloromethane. After the solution was stirred for 30 minutes, Compound 81 (0.1 g, 0.17 mmol) was added. The reaction mixture was stirred at room temperature for overnight. It was then diluted with dichloromethane (20 mL) and washed with a saturated sodium bicarbonate solution (2×20 mL). The organic layer was separated, dried over magnesium sulfate, filtered, and concentrated under vacuum. The crude product was purified by silica gel chromatography to give Compound 84 (0.1 g).

LC/MS (M+1)⁺: 682.3.

### EXAMPLE 85

Compound 85 was prepared following the procedures described below.

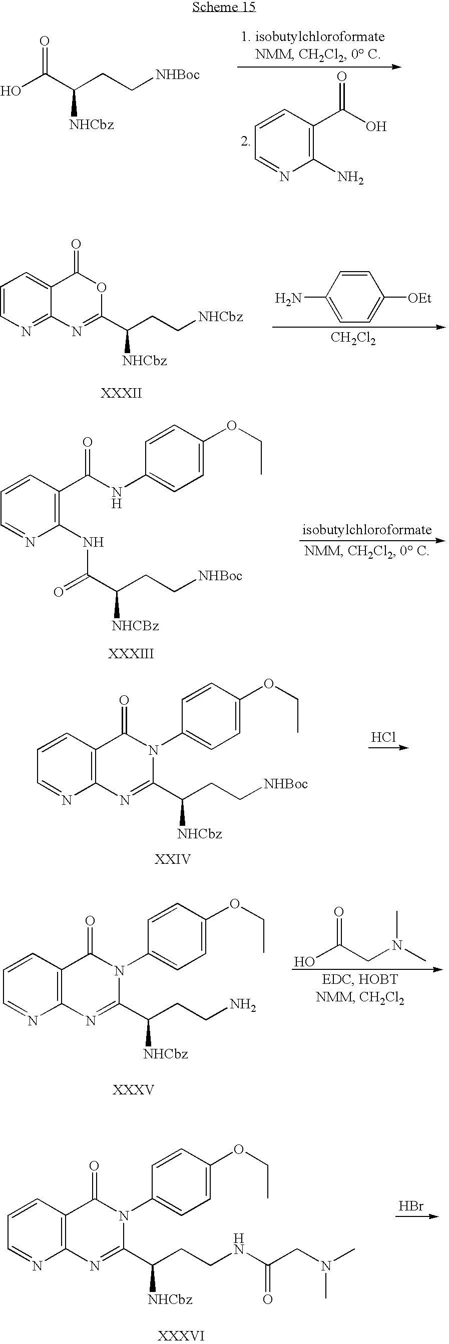

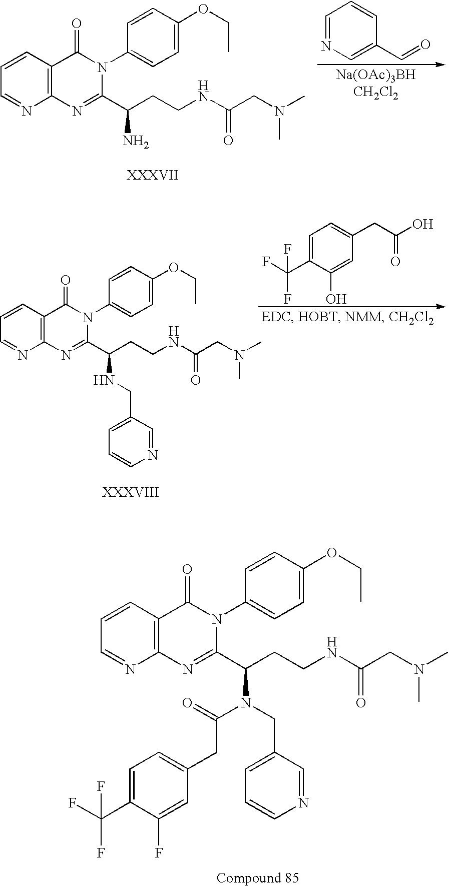

Intermediates XXXII, XXXIII, and XXXIV were prepared in a manner similar to that of Intermediates XXVII, XXVIII, and XXIX, respectively.

A mixture of Intermediate XXXIV (1.0 g, 1.7 mmol) and HCl in ether (15 mL) was stirred at room temperature for 4 hours. The solution was neutralized with 1N NaOH (30 mL) and extracted with ether (2×30 mL). The organic layer was separated, concentrated under vacuum, and dried to give Intermediate XXXV (0.77 g).

EDC (0.24 g, 1.26 mmol), HOBt (0.09 g, 0.63 mmol) and N-methyl morphorine (0.13 g, 1.28 mmol) were added to a solution of dimethylamino acetic acid (0.07 g, 0.5 mmol) in 5 mL dichloromethane. After the solution was stirred for 30 minutes, Intermediate XXXV (0.2 g, 0.42 mmol) was added. The reaction mixture was stirred at room temperature for overnight. It was then diluted with dichloromethane (10 mL) and washed with a saturated sodium bicarbonate solution (2×30 mL). The organic layer was separated, dried over magnesium sulfate, filtered, and concentrated under vacuum. The crude residue was purified by silica gel chromatography to give Intermediate XXXVI (0.2 g).

A mixture of Intermediate XXXVI (0.2 g, 0.36 mmol) and 33% HBr in HOAc (15 mL) was stirred at room temperature for 4 hours. The reaction mixture was then diluted with ether (25 mL), filtered, and washed with a mixture of MeOH and ether, and dried to give 0.12 g of Intermediate XXXVII.

To a solution of Intermediate XXXVII (0.34 g, 0.8 mmol) in 10 mL dichloromethane was added pyridine carboxaldehyde (0.08 g, 0.72 mmol) followed by sodium triacetoxy borohydride (0.21 g, 1 mmol). The reaction mixture was stirred at room temperature for overnight. It was then diluted with dichloromethane (10 mL) and washed with a 1.0 M ammonium hydroxide aqueous solution (10 mL). The organic layer was separated, dried over magnesium sulfate, filtered, and concentrated under vacuum. The crude residue was purified by silica gel chromatography to give 0.1 g of Intermediate XXXVIII.

EDC (0.05 g, 0.24 mmol), HOBt (0.016 g, 0.12 mmol), and N-methylmorphorine were added to a solution of (3-fluoro-4-trifluoromethyl-phenyl)-acetic acid (0.02 g, 0.09 mmol) in dichloromethane (3 mL). After the solution was stirred for 30 minutes, Intermediate XXXVIII (0.04 g, 0.08 mmol) was added. The reaction was stirred at room temperature for overnight. It was then diluted with dichloromethane (10 mL) and washed with a saturated sodium bicarbonate solution (2×20 mL). The organic layer was separated, dried over magnesium sulfate, filtered, and concentrated under vacuum. The crude product was purified by silica gel chromatography to give 0.02 g of Compound 85.

LC/MS (M+1)⁺: 720.3.

### EXAMPLE 86

Compound 86 was prepared in a manner similar to that described in Example 20.

LC/MS (M+1)⁺: 700.3.

### EXAMPLE 87

Compound 87 was prepared in a manner similar to the preparation of Intermediate XII described in Example 21.

LC/MS (M+1)⁺: 595.8.

### EXAMPLE 88

Compound 88 was prepared following the procedures described below.

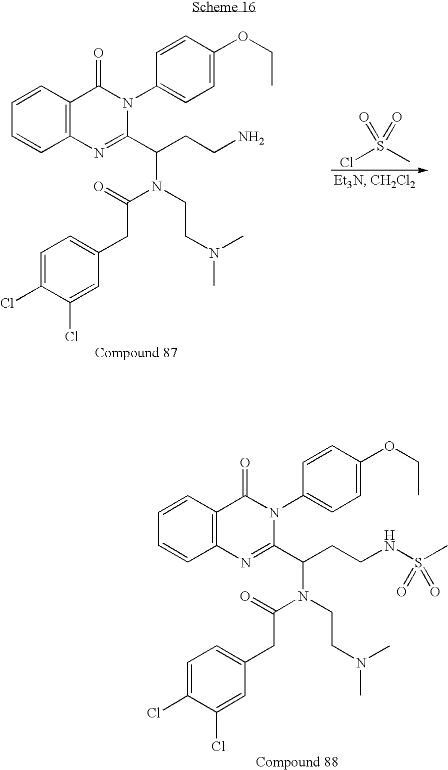

Methanesulfonyl chloride (0.03 g, 0.26 mmol) was added dropwise to a solution of Compound 87 (0.6 g, 0.17 mmol) and Et₃N (0.17 g, 1.7 mmol) in dichloromethane (10 mL) at 0° C. over a period of 10 minutes. The mixture was stirred at room temperature for overnight and then washed sequentially with 1 N HCl (10 mL) and water (20 mL). The organic layer was separated, dried over magnesium sulfate, filtered, and concentrated under vacuum. The crude product was purified by silica gel chromatography to give Compound 88 (0.09 g).

LC/MS (M+1)⁺: 687.5.

### EXAMPLE 89

Compound 89 was prepared following the procedures described below.

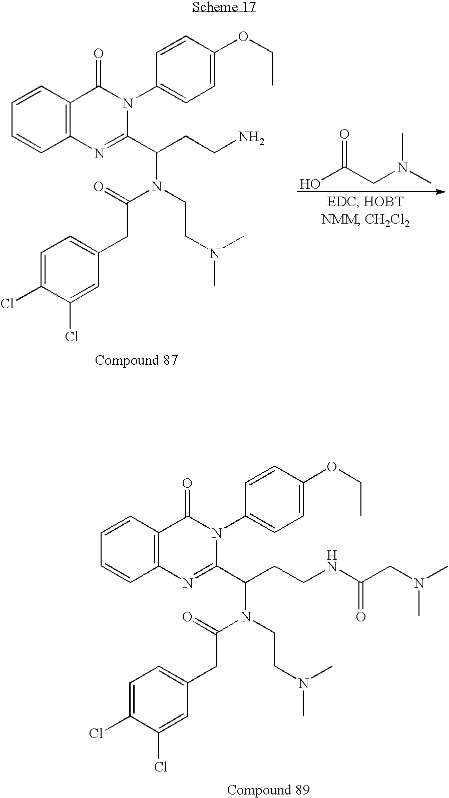

EDC (0.12 g, 0.63 mmol), HOBt (0.05 g, 0.32 mmol), and N-methyl morphorine (0.06 g, 0.6 mmol) were added to a solution of dimethylamino acetic acid (0.04 g, 0.25 mmol) in 5 mL dichloromethane. After the solution was stirred for 30 minutes, Compound 87 (0.1 g, 0.17 mmol) was added. The reaction mixture was stirred at room temperature for overnight. It was then diluted with dichloromethane (20 mL) and washed with a saturated sodium bicarbonate solution (2×20 mL). The organic layer was separated, dried over magnesium sulfate, filtrated, and concentrated under vacuum. The crude product was purified by silica gel chromatography to give 0.1 g of Compound 89.

LC/MS (M+1)⁺: 695.3.

### EXAMPLE 90

Compound 90 was prepared in a manner similar to that described in Example 87.

LC/MS (M+1)⁺: 638.2.

### EXAMPLE 91

Compound 91 was prepared in a manner similar to that described in Example 81.

LC/MS (M+1)⁺: 634.2.

### EXAMPLE 92

Compound 92 was prepared in a manner similar to that described in Example 83.

LC/MS (M+1)⁺: 719.3.

### EXAMPLE 93

Compound 93 was prepared in a manner similar to the preparation of Intermediate XII described in Example 21.

LC/MS (M+1)⁺: 684.3.

### EXAMPLE 94

Compound 94 was prepared following the procedures described below.

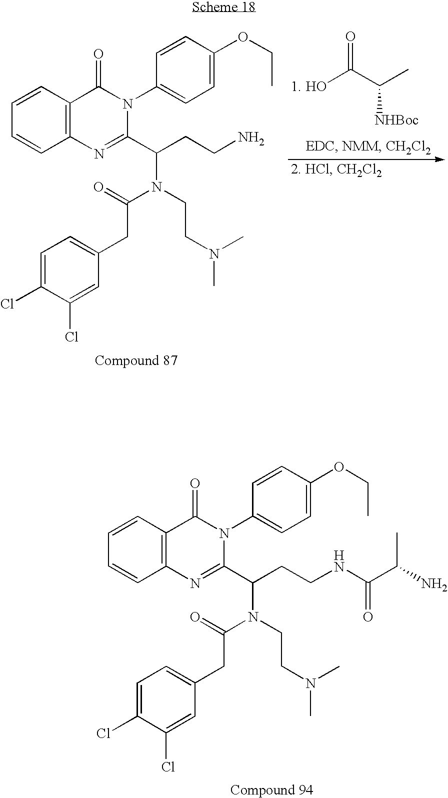

EDC (0.76 g, 3.96 mmol) and excess amount of N-methyl morphorine were added to a solution of Boc-L-alanine (0.56 g, 2.95 mmol) in dichloromethane (50 mL. After the solution was stirred for 30 minutes, Compound 87 (1.17 g, 1.97 mmol) was added. The reaction mixture was stirred at room temperature for overnight. It was then diluted with dichloromethane (200 mL) and washed with a saturated sodium bicarbonate solution (2×50 mL). The organic layer was separated, dried over magnesium sulfate, filtered, and concentrated under vacuum. The residue thus obtained was purified by silica gel chromatography to give white solid.

The solid was dissolved in dichloromethane (20 mL). 1N HCl in ether (30 mL) was then added. The solution was stirred at room temperature for 4 hours, concentrated under vacuum, washed with ether (10 mL), and dried under vacuum to give 0.8 g of compound 94.

LC/MS (M+1)⁺: 667.3.

### EXAMPLE 95

Compound 95 was prepared in a manner similar to the preparation of Intermediate XII described in Example 21.

LC/MS (M+1)⁺: 670.3.

### EXAMPLE 96

Compound 96 was prepared in a manner similar to the preparation of Intermediate XII described in Example 21.

LC/MS (M+1)⁺: 668.3.

### EXAMPLE 97

Compound 97 was prepared in a manner similar to the preparation of Intermediate XII described in Example 21.

LC/MS (M+1)⁺: 696.3.

### EXAMPLE 98

Compound 98 was prepared in a manner similar to that described in Example 91.

LC/MS (M+1)⁺: 648.3.

### EXAMPLE 99

Compound 99 was prepared in a manner similar to the preparation of Intermediate XII described in Example 21.

LC/MS (M+1)⁺: 656.3.

### EXAMPLE 100

Compound 100 was prepared in a manner similar to that described in Example 91.

LC/MS (M+1)⁺: 662.3.

### EXAMPLE 101

Compound 101 was prepared in a manner similar to that described in Example 20.

LC/MS (M+1)⁺: 699.9.

### EXAMPLE 102

Compound 102 was prepared in a manner similar to the preparation of Intermediate XII described in Example 21.

LC/MS (M+1)⁺: 614.3.

### EXAMPLE 103

Compound 103 was prepared in a manner similar to the preparation of Intermediate XII described in Example 21.

LC/MS (M+1)⁺: 656.3.

### EXAMPLE 104

Compound 104 was prepared in a manner similar to the preparation of Intermediate XII described in Example 21.

LC/MS (M+1)⁺: 668.3.

### EXAMPLE 105

Compound 105 was prepared in a manner similar to the preparation of Intermediate XII described in Example 21.

LC/MS (M+1)⁺: 642.3.

### EXAMPLE 106

Compound 106 was prepared in a manner similar to the preparation of Intermediate XII described in Example 21.

LC/MS (M+1)⁺: 697.3.

### EXAMPLE 107

Compound 107 was prepared in a manner similar to the preparation of Intermediate XII described in Example 21.

LC/MS (M+1)⁺: 642.3.

### EXAMPLE 108

Compound 108 was prepared in a manner similar to that described in Example 83.

LC/MS (M+1)⁺: 705.3.

### EXAMPLE 109

Compound 109 was prepared following the procedures described below.

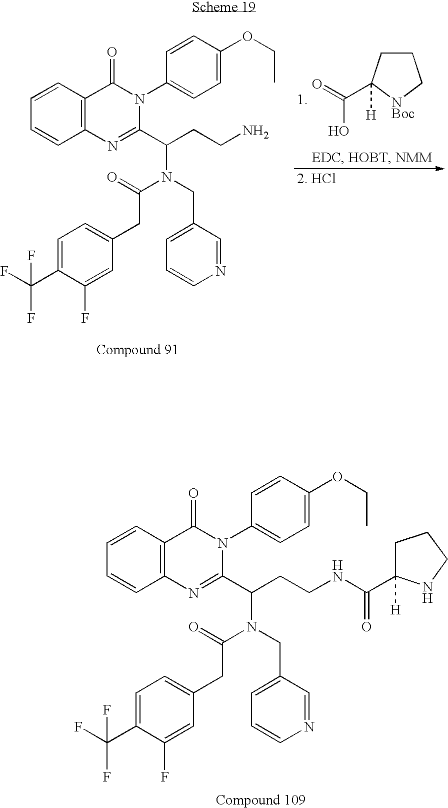

EDC (0.07 g, 0.38 mmol), HOBt (0.04 g, 0.28 mmol), and excess amount of N-methyl morphorine were added to a solution of Boc-L-prolin (0.07 g, 0.32 mmol) in 10 mL dichloromethane. After the solution was stirred for 30 minutes, Compound 91 (0.12 g, 0.19 mmol) was added. The reaction mixture was stirred at room temperature for overnight. It was then diluted with dichloromethane (20 mL) and washed with a saturated sodium bicarbonate solution (2×30 mL). The organic layer was separated, dried over magnesium sulfate, filtered, and concentrated under vacuum. The residue thus obtained was purified by silica gel chromatography to give a solid.

The solid was dissolved in dichloromethane (20 mL). 1N HCl in ether (30 mL) was added. The solution was stirred at room temperature for 4 hours, concentrated under vacuum, washed with ether (10 mL), and dried under vacuum to give 0.09 g of Compound 109.

LC/MS (M+1)⁺: 731.2.

### EXAMPLE 110

Compound 110 was prepared in a manner similar to the preparation of Intermediate XII described in Example 21.

LC/MS (M+1)⁺: 662.3.

### EXAMPLE 111

Compound 111 was prepared in a manner similar to that described in Example 94.

LC/MS (M+1)⁺: 765.9.

### EXAMPLE 112

Compound 112 was prepared in a manner similar to that described in Example 109.

LC/MS (M+1)⁺: 721.2.

### EXAMPLE 113

Compound 113 was prepared in a manner similar to that described in Example 82.

LC/MS (M+1)⁺: 791.6.

### EXAMPLE 114

Compound 114 was prepared following the procedures described below.

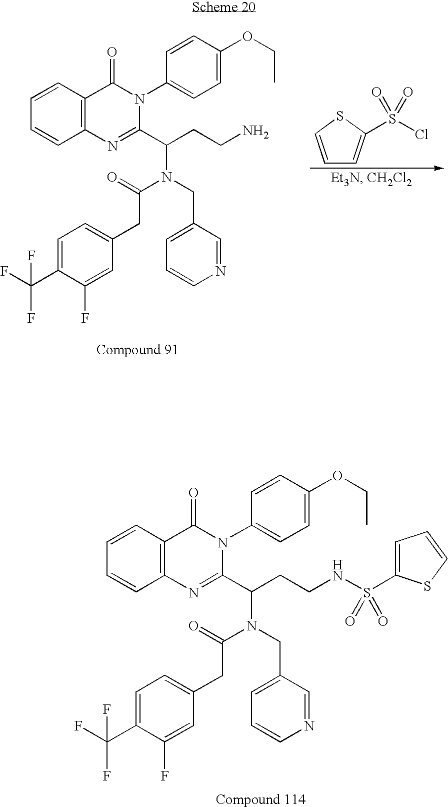

2-Thiophenesulfonyl chloride (0.04 g, 0.24 mmol) was added dropwise to a solution of Compound 91 (0.12 g, 0.19 mmol) and Et₃N (0.08 g, 0.8 mmol) in dichloromethane (5 mL) at 0° C. over a period of 10 minutes. The mixture was stirred at room temperature for overnight and then washed sequentially with 1 N HCl (10 mL) and water (20 mL). The organic layer was separated, dried over magnesium sulfate, filtered, and concentrated under vacuum. The crude product was purified by silica gel chromatography to give Compound 114 (0.09 g).

LC/MS (M+1)⁺: 780.2.

### EXAMPLE 115

Compound 115 was prepared in a manner similar to that described in Example 114.

LC/MS (M+1)⁺: 857.1.

### EXAMPLE 116

Compound 116 was prepared in a manner similar to that described in Example 114.

LC/MS (M+1)⁺: 793.1.

### EXAMPLE 117

Compound 117 was prepared following the procedures described below.

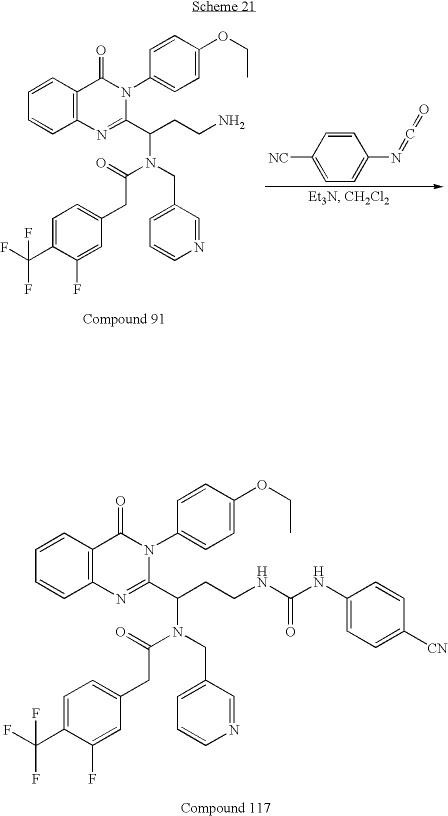

4-Cyanophenyl isocyanate (0.04 g, 0.29 mmol) was added dropwise to a solution of Compound 91 (0.14 g, 0.22 mmol) and Et₃N (0.04 g, 0.44 mmol) in dichloromethane (5 mL) at 0° C. over a period of 10 minutes. The mixture was stirred at room temperature for 3 hours and then washed sequentially with 1 N HCl (10 mL) and water (20 mL). The organic layer was separated, dried over magnesium sulfate, filtered, and concentrated under vacuum. The crude product was purified by silica gel chromatography to give compound 117 (0.1 g).

LC/MS (M+1)⁺: 778.3.

### EXAMPLE 118

Compound 118 was prepared in a manner similar to that described in Example 117.

LC/MS (M+1)⁺: 753.2.

### EXAMPLE 119

Compound 119 was prepared in a manner similar to that described in Example 82.

LC/MS (M+1)⁺: 801.4.

### EXAMPLE 120

Compound 120 was prepared in a manner similar to that described in Example 82.

LC/MS (M+1)⁺: 878.4.

### EXAMPLE 121

Compound 121 was prepared in a manner similar to that described in Example 82.

LC/MS (M+1)⁺: 734.3.

### EXAMPLE 122

Compound 122 was prepared in a manner similar to that described in Example 81.

LC/MS (M+1)⁺: 633.8.

### EXAMPLE 123

Compound 123 was prepared in a manner similar to the preparation of Intermediate XII described in Example 21.

LC/MS (M+1)⁺: 727.7.

### EXAMPLE 124

Compound 124 was prepared in a manner similar to that described in Example 114.

LC/MS (M+1)⁺: 876.3.

### EXAMPLE 125

Compound 125 was prepared in a manner similar to that described in Example 114.

LC/MS (M+1)⁺: 779.4.

### EXAMPLE 126

Compound 126 was prepared in a manner similar to that described in Example 82.

LC/MS (M+1)⁺: 799.6.

### EXAMPLE 127

Compound 127 was prepared in a manner similar to that described in Example 82.

LC/MS (M+1)⁺: 837.2.

### EXAMPLE 128

Compound 128 was prepared in a manner similar to that described in Example 82.

LC/MS (M+1)⁺: 857.2.

### EXAMPLE 129

Compound 129 was prepared following the procedures described below.

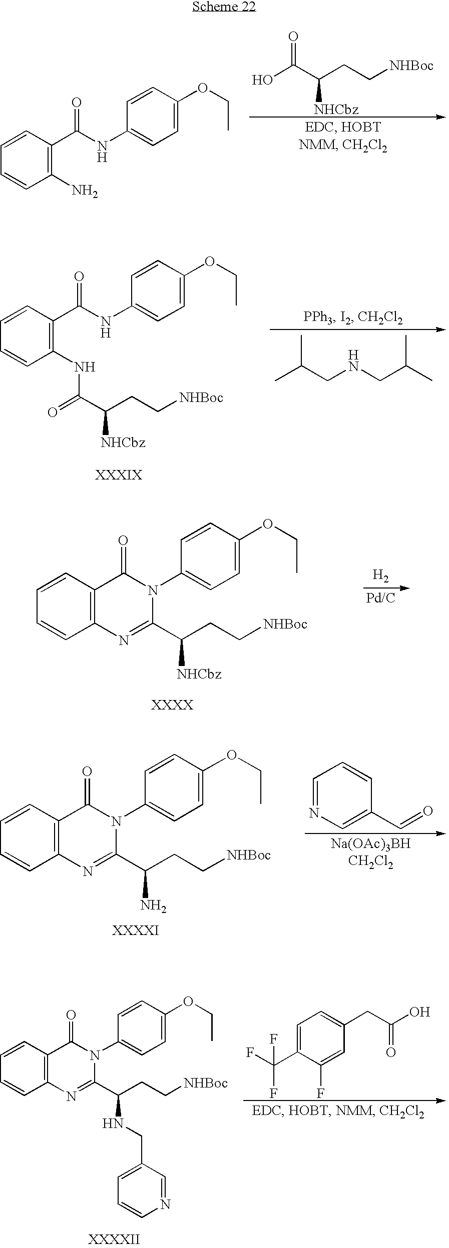

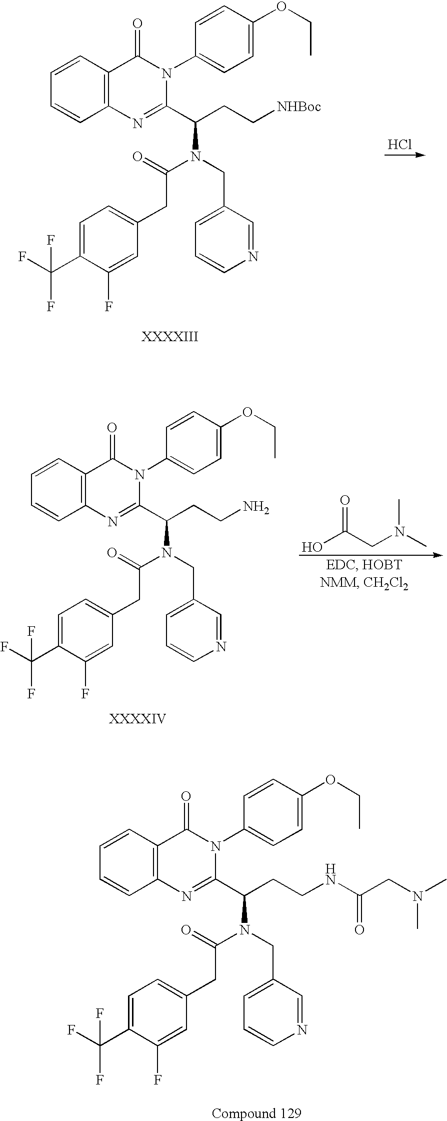

EDC (0.07 g, 0.38 mmol), HOBt (0.04 g, 0.28 mmol) and excess amount of N-methyl morphorine were added to a solution of 2-benzyloxycarbonylamino-4-tert-butoxycarbonylamino-butyric acid (0.11 g, 0.32 mmol) in 10 mL dichloromethane. After the solution was stirred for 30 minutes, 2-amino-N-(4-ethoxyphenyl)benzamide (0.082 g, 0.32 mmol) was added. The reaction mixture was stirred at room temperature for overnight. It was then filtered and washed with ether to give Intermediate XXXIX (0.14 g).

PPh₃ (1.4 g, 5.35 mmol), 12 (1.36 g, 5.35 mmol), and N,N-diisopropylethylamine (1.32 g, 10.2 mmol) were added to a solution of (3.0 g, 5.1 mmol) in 100 mL dichloromethane. The reaction was stirred at room temperature for overnight. The resulting solid was obtained by filtration and washed with ether to give Intermediate XXXX (2.18 g).

A catalytic amount of 10% Pd/C was added to a solution of Intermediate XXXX (2.0 g, 3.5 mmol) in 100 mL MeOH at H₂ atmosphere. The reaction mixture was stirred at room temperature for overnight. It was then filtered to remove the catalyst. The mixture thus obtained was concentrated under vacuum and purified by silica gel chromatography to give Intermediate XXXXI (1.38 g).

To a solution of Intermediate XXXXI (1.0 g, 2.3 mmol) in 50 mL dichloromethane was added pyridine-3-carboaldehyde (0.25 g, 2.3 mmol) followed by sodium triacetoxy borohydride (0.97 g, 4.6 mmol). The reaction mixture was stirred at room temperature for overnight. It was then diluted with dichloromethane (50 mL) and washed with a 1.0 M ammonium hydroxide aqueous solution (50 mL). The organic layer was separated, dried over magnesium sulfate, filtered, and concentrated under vacuum. The residue was purified by silica gel chromatography to give Intermediate XXXXII (0.97 g).

EDC (0.12 g, 0.63 mmol), HOBt (0.05 g, 0.32 mmol), and excess amount of N-methyl morphorine were added to a solution of (3-fluoro-4-trifluoromethyl-phenyl)-acetic acid (0.086 g, 0.25 mmol) in dichloromethane (10 mL) was added. After the solution was stirred for 30 minutes, Intermediate XXXXII (0.089 g, 0.17 mmol) was added. The reaction mixture was stirred at room temperature for overnight. It was then diluted with dichloromethane (20 mL) and washed with a saturated sodium bicarbonate solution (2×30 mL). The organic layer was separated, dried over magnesium sulfate, filtered, and concentrated under vacuum. The residue was purified by silica gel chromatography to give Intermediate XXXXIII (0.11 g).

A mixture of Intermediate XXXXIII (1.0 g, 1.4 mmol) and HCl in ether (25 mL) was stirred at room temperature for 4 hours. The mixture was concentrated under vacuum, washed with a 1N NaOH aqueous solution (15 mL), and extracted with ether (2×30 mL). The organic layer was separated, concentrated, and dried to give Intermediate XXXXIV (0.78 g).

EDC (0.12 g, 0.63 mmol), HOBt (0.05 g, 0.32 mmol), and excess amount of N-methyl morphorine were added to a solution of dimethylamino acetic acid (0.04 g, 0.25 mmol) in 10 mL dichloromethane was added. After the solution was stirred for 30 minutes, Intermediate XXXXIV (0.11 g, 0.17 mmol) was added. The reaction mixture was stirred at room temperature for overnight. It was then diluted with dichloromethane (30 mL) and washed with a saturated sodium bicarbonate solution (2×30 mL). The organic layer was separated, dried over magnesium sulfate, filtered, and concentrated under vacuum. The residue was purified by silica gel chromatography to give Compound 129 (0.095 g).

LC/MS (M+1)⁺: 718.7.

### EXAMPLE 130

Compound 130 was prepared in a manner similar to that described in Example 82.

LC/MS (M+1)⁺: 898.5.

### EXAMPLE 131

Compound 130 was prepared in a manner similar to that described in Example 129.

LC/MS (M+1)⁺: 841.6.

### EXAMPLE 132

Compound 132 was prepared in a manner similar to that described in Example 82.

LC/MS (M+1)⁺: 799.6.

### EXAMPLE 133

Compound 133 was prepared in a manner similar to the preparation of Intermediate XII described in Example 21.

LC/MS (M+1)⁺: 628.2.

### EXAMPLE 134

Compound 134 was prepared in a manner similar to the preparation of Intermediate XII described in Example 21.

LC/MS (M+1)⁺: 654.2.

### EXAMPLE 135

Compound 135 was prepared in a manner similar to the preparation of Intermediate XII described in Example 21.

LC/MS (M+1)⁺: 654.3.

### EXAMPLE 136

Compound 136 was prepared in a manner similar to that described in Example 82.

LC/MS (M+1)⁺: 810.2.

### EXAMPLE 137

Compound 137 was prepared in a manner similar to that described in Example 94.

LC/MS (M+1)⁺: 741.8.

### EXAMPLE 138

Compound 138 was prepared in a manner similar to that described in Example 94.

LC/MS (M+1)⁺: 755.8.

### EXAMPLE 139

Compound 139 was prepared in a manner similar to that described in Example 129.

LC/MS (M+1)⁺: 781.5.

### EXAMPLE 140

Compound 140 was prepared in a manner similar to that described in Example 114.

LC/MS (M+1)⁺: 846.5.

### EXAMPLE 141

Compound 141 was prepared in a manner similar to that described in Example 114.

LC/MS (M+1)⁺: 787.6.

### EXAMPLE 142

Compound 142 was prepared in a manner similar to that described in Example 114.

LC/MS (M+1)⁺: 840.4.

### EXAMPLE 143

Compound 143 was prepared in a manner similar to that described in Example 114.

LC/MS (M+1)⁺: 820.4.

### EXAMPLE 144

Compound 144 was prepared following the procedures described below.

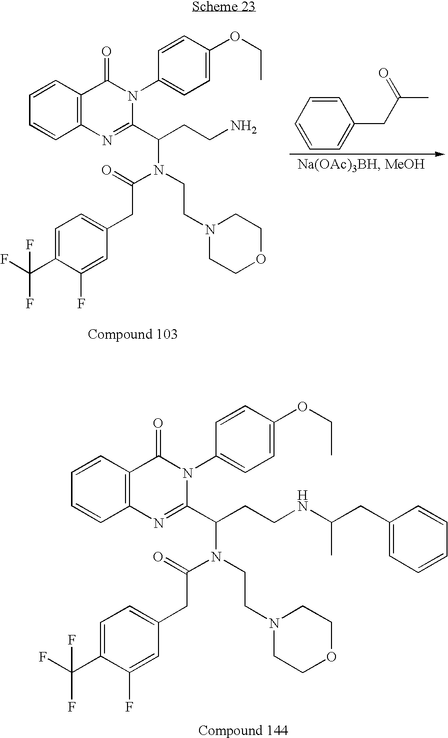

To a solution of Compound 103 (0.1 g, 0.15 mmol) in 30 mL methanol was added is 1-phenyl-propan-2-one (0.025 g, 0.17 mmol) followed by sodium triacetoxy borohydride (0.064 g, 0.3 mmol). The reaction mixture was stirred at room temperature for overnight. It was then diluted with dichloromethane (50 mL) and washed with a 1.0 M ammonium hydroxide aqueous solution (50 mL). The organic layer was dried over magnesium sulfate, filtered, and concentrated under vacuum. The crude product was purified by silica gel chromatography to give Compound 144 (0.098 g).

LC/MS (M+1)⁺: 773.8.

### EXAMPLE 145

Compound 145 was prepared in a manner similar to that described in Example 144.

LC/MS (M+1)⁺: 780.8.

### EXAMPLE 146

Compound 146 was prepared in a manner similar to that described in Example 144.

LC/MS (M+1)⁺: 738.6.

### EXAMPLE 147

Compound 147 was prepared in a manner similar to that described in Example 129.

LC/MS (M+1)⁺: 752.7.

### EXAMPLE 148

Compound 148 was prepared in a manner similar to that described in Example 117.

LC/MS (M+1)⁺: 805.3.

### EXAMPLE 149

Compound 149 was prepared in a manner similar to the preparation of Intermediate XII described in Example 21.

LC/MS (M+1)⁺: 632.3.

### EXAMPLE 150

Compound 150 was prepared in a manner similar to that described in Example 82.

LC/MS (M+1)⁺: 712.7.

### EXAMPLE 151

Compound 151 was prepared in a manner similar to that described in Example 82.

LC/MS (M+1)⁺: 738.7.

### EXAMPLE 152

Compound 152 was prepared in a manner similar to that described in Example 82.

LC/MS (M+1)⁺: 692.3.

### EXAMPLE 153

Compound 153 was prepared in a manner similar to that described in Example 82.

LC/MS (M+1)⁺: 699.4.

### EXAMPLE 154

Compound 154 was prepared in a manner similar to that described in Example 94.

LC/MS (M+1)⁺: 804.4.

### EXAMPLE 155

Compound 155 was prepared in a manner similar to that described in Example 94.

LC/MS (M+1)⁺: 728.4.

### EXAMPLE 156

Compound 156 was prepared in a manner similar to the preparation of Intermediate XII described in Example 21.

LC/MS (M+1)⁺: 598.4.

### EXAMPLE 157

Compound 157 was prepared in a manner similar to the preparation of Intermediate XII described in Example 21.

LC/MS (M+1)⁺: 584.4.

### EXAMPLE 158

Compound 158 was prepared in a manner similar to that described in Example 82.

LC/MS (M+1)⁺: 674.0.

### EXAMPLE 159

Compound 159 was prepared in a manner similar to that described in Example 82.

LC/MS (M+1)⁺: 758.0.

### EXAMPLE 160

Compound 160 was prepared in a manner similar to that described in Example 82.

LC/MS (M+1)⁺: 688.0.

### EXAMPLE 161

Compound 161 was prepared in a manner similar to that described in Example 82.

LC/MS (M+1)⁺: 681.1.

### EXAMPLE 162

Compound 162 was prepared in a manner similar to the preparation of Intermediate XII described in Example 21.

LC/MS (M+1)⁺: 570.0.

### EXAMPLE 163

Compound 163 was prepared in a manner similar to that described in Example 144.

LC/MS (M+1)⁺: 756.1.

### EXAMPLE 164

Compound 164 was prepared in a manner similar to that described in Example 94.

LC/MS (M+1)⁺: 743.1.

### EXAMPLE 165

Compound 165 was prepared in a manner similar to that described in Example 144.

LC/MS (M+1)⁺: 759.1.

### EXAMPLE 166

Compound 166 was prepared in a manner similar to that described in Example 144.

LC/MS (M+1)⁺: 717.1.

### EXAMPLE 167

Compound 167 was prepared in a manner similar to that described in Example 144.

LC/MS (M+1)⁺: 681.3.

### EXAMPLE 168

Compound 168 was prepared in a manner similar to that described in Example 144.

LC/MS (M+1)⁺: 670.2.

### EXAMPLE 169

Compound 169 was prepared in a manner similar to that described in Example 144.

LC/MS (M+1)⁺: 652.9.

### EXAMPLE 170

Compound 170 was prepared in a manner similar to that described in Example 144.

LC/MS (M+1)⁺: 718.3.

### EXAMPLE 171

Compound 171 was prepared in a manner similar to that described in Example 144.

LC/MS (M+1)⁺: 707.3.

### EXAMPLE 172

Compound 172 was prepared in a manner similar to that described in Example 144.

LC/MS (M+1)⁺: 679.2.

### EXAMPLE 173

Compound 173 was prepared in a manner similar to that described in Example 114.

LC/MS (M+1)⁺: 803.8.

### EXAMPLE 174

Compound 174 was prepared in a manner similar to that described in Example 114.

LC/MS (M+1)⁺: 825.8.

### EXAMPLE 175

Compound 175 was prepared in a manner similar to the preparation of Intermediate XII described in Example 21.

LC/MS (M+1)⁺: 588.0.

### EXAMPLE 176

Compound 176 was prepared in a manner similar to that described in Example 144.

LC/MS (M+1)⁺: 699.2.

### EXAMPLE 177

Compound 177 was prepared in a manner similar to that described in Example 144.

LC/MS (M+1)⁺: 713.3.

### EXAMPLE 178

Compound 178 was prepared in a manner similar to that described in Example 144.

LC/MS (M+1)⁺: 672.2.

### EXAMPLE 179

Compound 179 was prepared in a manner similar to that described in Example 144.

LC/MS (M+1)⁺: 688.2.

### EXAMPLE 180

Compound 180 was prepared in a manner similar to that described in Example 144.

LC/MS (M+1)⁺: 753.2.

### EXAMPLE 181

Compound 181 was prepared in a manner similar to that described in Example 144.

LC/MS (M+1)⁺: 753.3.

### EXAMPLE 182

Compound 182 was prepared in a manner similar to that described in Example 144.

LC/MS (M+1)⁺: 712.2.

### EXAMPLE 183

Compound 183 was prepared in a manner similar to that described in Example 144.

LC/MS (M+1)⁺: 719.2.

### EXAMPLE 184

Compound 184 was prepared in a manner similar to that described in Example 144.

LC/MS (M+1)⁺: 733.2.

### EXAMPLE 185

Compound 185 was prepared in a manner similar to the preparation of Intermediate XII described in Example 21.

LC/MS (M+1)⁺: 686.2.

### EXAMPLE 186

Compound 186 was prepared in a manner similar to that described in Example 129.

LC/MS (M+1)⁺: 757.3.

### EXAMPLE 187

Compound 187 was prepared from compound 186 in a manner similar to the preparation of Intermediate XXXV described in Example 85.

LC/MS (M+1)⁺: 657.3.

### EXAMPLE 188

Compound 188 was prepared from compound 187 in a manner similar to the preparation of Intermediate XXXXI described in Example 129.

LC/MS (M+1)⁺: 572.2.

### EXAMPLE 189

Compounds 1-188 were tested for their efficacy in blocking activation of CXCR3 using a DELFIA GTP-binding kit (Wallac Oy, Turku, Finland). The DELFIA GTP-binding assay is a time-resolved fluorometric assay based on GDP-GTP exchange on G-protein subunits followed by activation of a G protein-coupled receptor by its agonists. Eu-GTP, obtained from Wallac Oy, was used in this assay to allow monitoring of agonist-dependent activation of G-protein. Stimulation of CXCR3 by interferon-a inducible protein 10 (IP-10) leads to the replacement of GDP by GTP on the a-subunit of G-protein. This GTP-Gα complex represents the activated form of G-protein. Eu-GTP, a non-hydrolysable analog of GTP, can be used to quantify the amount of activated G-protein. (Peltonen et al., Eur. J. Pharmacol. (1998) 355:275.) Plasma membrane of CXCR3-expressing HEK293 cells was suspended in an assay buffer (50 mM NaCl, 100 μg/mL saponin, 3 mM MgCl₂, 3 μM GDP, 5% BSA, 50 mM HEPES, pH 7.4). An aliquot (4 μg protein) was added to each well of an AcroPlate (Pall Life Sciences, Ann Arbor, Mich.). After the addition of the test compounds (10 μM in 0.1% DMSO) and IP-10 (4 nM in the assay buffer), the assay plate was incubated in the dark at room temperature with slow shaking for 10 minutes. Eu-GTP was added to each well and the plate was incubated again for 60 minutes. The assay was terminated by washing the plate twice with a wash solution provided in the assay kit. Binding of Eu-GTP was determined based on the fluorescence signal from a Victor 2 multi-label reader.

Unexpectedly, 138 compounds showed IC₅₀ values lower than 1 μM, 37 compounds showed IC₅₀ values between 1 μM and 10 μM, and 13 compounds showed IC₅₀ values greater than 10 μM.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A compound of formula (I): 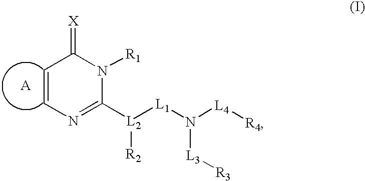
wherein
A is aryl or heteroaryl;
X is S or NRₐ₁;
L₁ is —C(R_{b1}R_{b2})—, C₂-C₁₀ alkylene, C₂-C₁₀ heteroalkylene, or deleted;
L₂ is 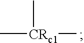
or L₂ and R₂ together are deleted;
each of L₃ and L₄, independently, is —C(O)—, —SO₂—, —C(O)O—, —C(O)NR_{d1}—, —C(O)CH₂—, —CH₂C(O)—, —SO₂CH₂—, —CH₂SO₂—, C₁-C₁₀ alkylene, or C₁-C₁₀ heteroalkylene;
or L₃, L₄, and the nitrogen atom to which they are both attached, together are C₅-C₇ heterocycloalkyl or heteroaryl; or L₁, L₃, and the nitrogen atom to which they are both attached, together are C₅-C₇ heterocycloalkyl or heteroaryl; or L₁, L₄, and the nitrogen atom to which they are both attached, together are C₅-C₇ heterocycloalkyl or heteroaryl;
R₁ is H, C₁-C₁₀ alkyl, C₃-C₂₀ cycloalkyl, C₃-C₂₀ heterocycloalkyl, aryl, or heteroaryl;
R₂ is H, C₁-C₁₀ alkyl, C₃-C₂₀ cycloalkyl, C₃-C₂₀ heterocycloalkyl, aryl, heteroaryl, or ORₑ₁; or R₂ and L₂ together are deleted; and
each of R₃ and R₄, independently, is C₁-C₁₀ alkyl, C₃-C₂₀ cycloalkyl, C₃-C₂₀ heterocycloalkyl, aryl, heteroaryl, halo, cyano, amidino, guanidine, ureido, OR_{f1}, NR_{f1}R_{f2}, C(O)NR_{f1}R_{f2}, N(R_{f1})—C(O)R_{f2}, N(R_{f1})—C(O)OR_{f1}, C(O)Rn, N(R_{f1})—C(S)NR_{f2}R_{f1}, N(R_{f1})—C(NR_{f2})—NR_{f1}R_{f4}, or N(R_{f1})—C(NR_{f2})—SR_{f1};
in which each of Rₐ₁, R_{b1}, R_{b2}, R_{c1}, R_{d1}, Rₑ₁, R_{f1}, R_{f2}, R_{f3}, and R_{f4}, independently, is H, C₁-C₁₀ alkyl, C₃-C₂₀ cycloalkyl, C₃-C₂₀ heterocycloalkyl, aryl, heteroaryl, cyano, OR, COOR, or C(O)NH₂; or R_{b1}, R_{b2}, and the carbon atom to which they are both attached, together are C₃-C₈ cycloalkyl or C₃-C₈ heterocycloalkyl; or R_{c1}, R₂, and the carbon atom to which they are both attached, together are C₃-C₈ cycloalkyl or C₃-C₈ heterocycloalkyl; R being H or C₁-C₁₀ alkyl.

2. The compound of claim 1, wherein X is S; L₁ is deleted; L₂ is 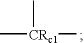
each of L₃ and L₄, independently, is —C(O)— or C₁-C₁₀ alkylene; R₁ is aryl; R₂ is C₁-C₁₀ alkyl; and each of R₃ and R₄, independently, is C₁-C₁₀ alkyl, C₃-C₂₀ heterocycloalkyl, heteroaryl, or NR_{f1}R_{f2}.

3. The compound of claim 2, wherein A is phenyl or thienyl.

4. The compound of claim 3, wherein each of L₃ and L₄, independently, is —C(O)—, —CH₂—, —(CH₂)₂—, or —(CH₂)₃—.

5. The compound of claim 4, wherein R₁ is phenyl substituted with F, OCH₃, or OCH₂CH₃, and R₂ is methyl.

6. The compound of claim 5, wherein one of R₃ and R₄ is methyl substituted with phenyl, in which the phenyl is further substituted with F, Cl, CF₃, or phenyl; and the other of R₃ and R₄ is C₃-C₂₀ heterocycloalkyl, heteroaryl, or NR_{f1}R_{f2}.

7. The compound of claim 6, wherein the compound is one of compounds 4, 6, 12, and 15-19.

8. A method for treating an inflammatory or immune disease, comprising administering to a subject in need thereof an effective amount of a compound of claim 1.

9. The method of claim 8, wherein the inflammatory or immune disease is selected from the group consisting of neurodegenerative disease, multiple sclerosis, systemic lupus erythematosus, rheumatoid arthritis, ankylosing spondylitis, psoriatic arthritis, juvenile rheumatoid arthritis, atherosclerosis, vasculitis, chronic heart failure, cerebrovascular ischemia, encephalitis, meningitis, hepatitis, nephritis, sepsis, sarcoidosis, psoriasis, eczema, uticaria, type I diabetes, asthma, conjunctivitis, otitis, allergic rhinitis, chronic obstructive pulmonary disease, sinusitis, dermatitis, inflammatory bowel disease, ulcerative colitis, Crohn's disease, Behcet's syndrome, pulmonary fibrosis, endometriosis, gout, cancer, cachexia, a viral infection, a bacterial infection, an organ transplant condition, a skin transplant condition, and a graft versus host disease.

10. The method of claim 9, wherein the neurodegenerative disease is Alzheimer's disease.

11. The method of claim 8, wherein the compound is concurrently administered in combination with a second therapeutic agent.

12. A pharmaceutical composition, comprising a compound of claim 1 and a pharmaceutically acceptable carrier.

13. The composition of claim 12, further comprising a second therapeutic agent.

14. A compound of formula (I): 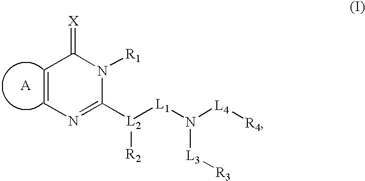
wherein
A is aryl or heteroaryl;
X is O, S, or NRₐ₁;
L₁ is —C(R_{b1}R_{b2})—, C₂-C₁₀ alkylene, C₂-C₁₀ heteroalkylene, or deleted;
L₂ is 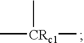
each of L₃ and L₄, independently, is —C(O)—, —SO₂—, —C(O)O—, —C(O)NR_{d1}—, —C(O)CH₂—, —CH₂C(O)—, —SO₂CH₂—, —CH₂SO₂—, C₁-C₁₀ alkylene, or C₁-C₁₀ heteroalkylene;
or L₃, L₄, and the nitrogen atom to which they are both attached, together are C₅-C₇ heterocycloalkyl or heteroaryl; or L₁, L₃, and the nitrogen atom to which they are both attached, together are C₅-C₇ heterocycloalkyl or heteroaryl; or L₁, L₄, and the nitrogen atom to which they are both attached, together are C₅-C₇ heterocycloalkyl or heteroaryl;
R₁ is H, C₁-C₁₀ alkyl, C₃-C₂₀ cycloalkyl, C₃-C₂₀ heterocycloalkyl, aryl, or heteroaryl;
R₂ is C₃-C₂₀ cycloalkyl, C₃-C₂₀ heterocycloalkyl, aryl, heteroaryl, ORₑ₁, or C₁-C₁₀ alkyl or C₁-C₁₀ heteroalkyl substituted with NRₑ₁Rₑ₂, N(Rₑ₁)—C(O)Rₑ₂, N(Rₑ₁)—C(O)ORₑ₂, N(Rₑ₁)—C(O)NRₑ₂Rₑ₃, N(Rₑ₁)—SO₂Rₑ₂, N(Rₑ₁)—C(S)NRₑ₂Rₑ₃, N(Rₑ₁)—C(NRₑ₂)—NRₑ₃Rₑ₄, or N(Rₑ₁)—C(NRₑ₂)—SRₑ₃; and
each of R₃ and R₄, independently, is C₁-C₁₀ alkyl, C₃-C₂₀ cycloalkyl, C₃-C₂₀ heterocycloalkyl, aryl, heteroaryl, halo, cyano, amidino, guanidine, ureido, OR_{f1}, NR_{f1}R_{f2}, C(O)NR_{f1}R_{f2}, N(R_{f1})—C(O)R_{f2}, N(R_{f1})—C(O)OR_{f2}, C(O)R_{f1}, N(R_{f1})—C(S)NR_{f2}R_{f3}, N(R_{f1})—C(NR_{f2})—NR_{f3}R_{f4}, or N(R_{f1})—C(NR_{f1})—SR_{f3};
in which each of Rₐ₁, R_{b1}, R_{b2}, R_{c1}, R_{d1}, Rₑ₁, Rₑ₂, Rₑ₃, Rₑ₄, R_{f1}, R_{f2}, R_{f3}, and R_{f4}, independently, is H, C₁-C₁₀ alkyl, C₃-C₂₀ cycloalkyl, C₃-C₂₀ heterocycloalkyl, aryl, heteroaryl, cyano, OR, COOR, or C(O)NH₂; or R_{b1}, R_{b2}, and the carbon atom to which they are both attached, together are C₃-C₈ cycloalkyl or C₃-C₈ heterocycloalkyl; or R_{c1}, R₂, and the carbon atom to which they are both attached, together are C₃-C₈ cycloalkyl or C₃-C₈ heterocycloalkyl; R being H or C₁-C₁₀ alkyl.

15. The compound of claim 14, wherein X is O; L₁ is deleted; each of L₃ and L₄, independently, is —C(O)— or C₁-C₁₀ alkylene; R₁ is aryl; R₂ is C₁-C₁₀ alkyl substituted with Nₑ₁Rₑ₂, N(Rₑ₁)—C(O)Rₑ₂, N(Rₑ₁)C(O)ORₑ₂, N(Rₑ₁)—C(O)Nₑ₂Rₑ₃, N(Rₑ₁)—SO₂Rₑ₂, or N(Rₑ₁)—C(NRₑ₂)—SRₑ₃; and each of R₃ and R₄, independently, is C₁-C₁₀ alkyl, C₃-C₂₀ heterocycloalkyl, heteroaryl, NR_{f1}R_{f2}, N(R_{f1})—C(O)R_{f2}, or N(R_{f1})—C(O)OR_{f1}.

16. The compound of claim 15, wherein A is phenyl or pyridyl.

17. The compound of claim 16, wherein each of L₃ and L₄, independently, is —C(O)—, —CH₂—, —(CH₂)₂—, or —(CH₂)₃—.

18. The compound of claim 17, wherein R₁ is phenyl substituted with F, OCH₃, or OCH₂CH₃.

19. The compound of claim 18, wherein one of R₃ and R₄ is C₁-C₁₀ alkyl optionally substituted with phenyl, in which the phenyl is further substituted with F, Cl, or CF₃; and the other of R₃ and R₄ is C₃-C₂₀ heterocycloalkyl, heteroaryl, NR_{f1}R_{f2}, N(R_{f1})—C(O)R₂, or N(R_{f1})—C(O)OR_{f2}.

20. The compound of claim 19, wherein the compound is one of compounds 20, 22-29, 31, 33-36, 38, 39, 42, 43, 81, 84-87, 89-140, and 144-185.

21. A method for treating an inflammatory or immune disease, comprising administering to a subject in need thereof an effective amount of a compound of claim 14.

22. The method of claim 21, wherein the inflammatory or immune disease is selected from the group consisting of neurodegenerative disease, multiple sclerosis, systemic lupus erythematosus, rheumatoid arthritis, ankylosing spondylitis, psoriatic arthritis, juvenile rheumatoid arthritis, atherosclerosis, vasculitis, chronic heart failure, cerebrovascular ischemia, encephalitis, meningitis, hepatitis, nephritis, sepsis, sarcoidosis, psoriasis, eczema, uticaria, type I diabetes, asthma, conjunctivitis, otitis, allergic rhinitis, chronic obstructive pulmonary disease, sinusitis, dermatitis, inflammatory bowel disease, ulcerative colitis, Crohn's disease, Behcet's syndrome, pulmonary fibrosis, endometriosis, gout, cancer, cachexia, a viral infection, a bacterial infection, an organ transplant condition, a skin transplant condition, and a graft versus host disease.

23. The method of claim 22, wherein the neurodegenerative disease is Alzheimer's disease.

24. The method of claim 21, wherein the compound is concurrently administered in combination with a second therapeutic agent.

25. A pharmaceutical composition, comprising a compound of claim 14 and a pharmaceutically acceptable carrier.

26. The composition of claim 25, further comprising a second therapeutic agent.

27. A compound of formula (I): 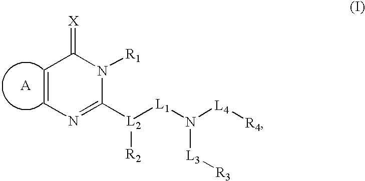
wherein
A is aryl or heteroaryl;
X is O, S, or NRₐ₁;
L₁ is —C(R_{b1}R_{b2})—, C₂-C₁₀ alkylene, or C₂-C₁₀ heteroalkylene;
L₂ is 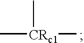
each of L₃ and L₄, independently, is —C(O)—, —SO₂—, —C(O)O—, —C(O)NR_{d1}—, —C(O)CH₂—, —CH₂C(O)—, —SO₂CH₂—, —CH₂SO₂—, C₁-C₁₀ alkylene, or C₁-C₁₀ heteroalkylene;
or L₃, L₄, and the nitrogen atom to which they are both attached, together are C₅-C₇ heterocycloalkyl or heteroaryl; or L₁, L₃, and the nitrogen atom to which they are both attached, together are C₅-C₇ heterocycloalkyl or heteroaryl; or L₁, L₄, and the nitrogen atom to which they are both attached, together are C₅-C₇ heterocycloalkyl or heteroaryl;
R₁ is H, C₁-C₁₀ alkyl, C₃-C₂₀ cycloalkyl, C₃-C₂₀ heterocycloalkyl, aryl, or heteroaryl;
R₂ is H, C₁-C₁₀ alkyl, C₃-C₂₀ cycloalkyl, C₃-C₂₀ heterocycloalkyl, aryl, heteroaryl, or L₂′-R₂′; L₂′ being —N(Rₑ₁)—, —C(O)—, —SO₂—, —C(O)O—, —C(O)NRₑ₁—, —C(O)CH₂—, —CH₂C(O)—, —SO₂CH₂—, —CH₂SO₂—, C₁-C₁₀ alkylene, or C₁-C₁₀ heteroalkylene; R₂′ being H, C₁-C₁₀ alkyl, C₃-C₂₀ cycloalkyl, C₃-C₂₀ heterocycloalkyl, aryl, heteroaryl, halo, cyano, amidino, guanidine, ureido, ORₑ₂, NRₑ₂Rₑ₃, C(O)NRₑ₂Rₑ₃, N(Rₑ₂)—C(O)Rₑ₃, N(Rₑ₂)—C(O)ORₑ₃, C(O)Rₑ₂, N(Rₑ₂)—C(S)NRₑ₃Rₑ₄, N(Rₑ₂)—C(Rₑ₃)—NRₑ₄Rₑ₅, or N(Rₑ₂)—C(NRₑ₃)—SRₑ₄; and
each of R₃ and R₄, independently, is C₁-C₁₀ alkyl, C₃-C₂₀ cycloalkyl, C₃-C₂₀ heterocycloalkyl, aryl, heteroaryl, halo, cyano, amidino, guanidine, ureido, OR_{f1}, NR_{f1}R_{f2}, C(O)NR_{f1}R_{f2}, N(R_{f1})—C(O)R_{f2}, N(R_{f1})—C(O)OR_{f2}, C(O)R_{f1}, N(R_{f1})—C(S)NR_{f2}R_{f3}, N(R_{f1})—C(NR_{f2})—NR_{f3}R_{f4}, or N(R_{f1})—C(NR_{f2})—SR_{f3};
in which each of Rₐ₁, R_{b1}, R_{b2}, R_{c1}, R_{d1}, Rₑ₁, Rₑ₂, Rₑ₃, Rₑ₄, Rₑ₅, R_{f1}, R_{f2}, R_{f3}, and R_{f4}, independently, is H, C₁-C₁₀ alkyl, C₃-C₂₀ cycloalkyl, C₃-C₂₀ heterocycloalkyl, aryl, heteroaryl, cyano, OR, COOR, or C(O)NH₂; or R_{b1}, R_{b2}, and the carbon atom to which they are both attached, together are C₃-C₈ cycloalkyl or C₃-C₈ heterocycloalkyl; or R_{c1}, R₂, and the carbon atom to which they are both attached, together are C₃-C₈ cycloalkyl or C₃-C₈ heterocycloalkyl; R being H or C₁-C₁₀ alkyl.

28. The compound of claim 27, wherein A is aryl; X is O; L₁ is —C(R_{b1}R_{b2})—; each of L₃ and L₄, independently, is —C(O)—, —SO₂—, or C₁-C₁₀ alkylene; R₁ is aryl; R₂ is H or L₂′-R₂′, L₂′ being —N(Rₑ₁)— or C₁-C₁₀ heteroalkylene and R₂′ being H, NRₑ₂Rₑ₃, or C(O)Rₑ₂; and each of R₃ and R₄, independently, is C₁-C₁₀ alkyl, C₃-C₂₀ heterocycloalkyl, aryl, heteroaryl, NR_{f1}R_{f2}, C(O)NR_{f1}R_{f2}, N(R_{f1})—C(O)OR_{f2}, or N(R_{f1})—C(NR_{f2})—SR_{f3}.

29. The compound of claim 28, wherein A is phenyl.

30. The compound of claim 29, wherein each of L₃ and L₄, independently, is —C(O)—, —SO₂—, —CH₂—, —(CH₂)₂—, or —(CH₂)₃—.

31. The compound of claim 30, wherein R₁ is phenyl substituted with OCH₃ or OCH₂CH₃ and R₂ is H, NH₂, OCH₂CH₂N(CH₃)₂, or NHC(O)CH₂N(CH₃)₂.

32. The compound of claim 31, wherein one of R₃ and R₄ is phenyl substituted with OCH₃ or methyl substituted with phenyl, in which the phenyl is further substituted with F, Cl, or CF₃; and the other of R₃ and R₄ is C₃-C₂₀ heterocycloalkyl, heteroaryl, NR_{f1}R_{f2}, C(O)NR_{f1}R_{f2}, N(R_{f1})—C(O)OR_{f2}, or N(R_{f1})—C(NR_{f2})—SR_{f3}.

33. The compound of claim 32, wherein the compound is one of compounds 45, 49, 58, 61, 63, 72, 74, 77, and 186-188.

34. A method for treating an inflammatory or immune disease, comprising administering to a subject in need thereof an effective amount of a compound of claim 27.

35. The method of claim 34, wherein the inflammatory or immune disease is selected from the group consisting of neurodegenerative disease, multiple sclerosis, systemic lupus erythematosus, rheumatoid arthritis, ankylosing spondylitis, psoriatic arthritis, juvenile rheumatoid arthritis, atherosclerosis, vasculitis, chronic heart failure, cerebrovascular ischemia, encephalitis, meningitis, hepatitis, nephritis, sepsis, sarcoidosis, psoriasis, eczema, uticaria, type I diabetes, asthma, conjunctivitis, otitis, allergic rhinitis, chronic obstructive pulmonary disease, sinusitis, dermatitis, inflammatory bowel disease, ulcerative colitis, Crohn's disease, Behcet's syndrome, pulmonary fibrosis, endometriosis, gout, cancer, cachexia, a viral infection, a bacterial infection, an organ transplant condition, a skin transplant condition, and a graft versus host disease.

36. The method of claim 35, wherein the neurodegenerative disease is Alzheimer's disease.

37. The method of claim 34, wherein the compound is concurrently administered in combination with a second therapeutic agent.

38. A pharmaceutical composition, comprising a compound of claim 27 and a pharmaceutically acceptable carrier.

39. The composition of claim 38, further comprising a second therapeutic agent.
